(19) United States
(12) Patent Application Publication
(10) Pub. No.: US 2018/0362625 A1
(43) Pub. Date: Dec. 20, 2018

(54) REGULATION OF CYTOKINE PRODUCTION

(71) Applicant: Commonwealth Scientific and Industrial Research Organisation, Acton, Australian Capital Territory (AU)

(72) Inventors: Cameron STEWART, Belmont, Victoria (AU); Rebecca AMBROSE, East Geelong, Victoria (AU); Andrew BEAN, Ocean Grove, Victoria (AU)

(21) Appl. No.: 15/781,322

(22) PCT Filed: Dec. 2, 2016
(86) PCT No.: PCT/AU2016/051192

§ 371 Date: Jun. 4, 2018

(30) Foreign Application Priority Data
Dec. 4, 2015 (AU) 2015905035

(51) Int. Cl. C07K 16/18; A61K 39/395; A61K 45/06; C07K 14/47; C12N 15/113; A61K 31/713; G01N 33/68; A61P 31/14

(52) U.S. Cl. CPC C07K 16/18; A61K 39/3955; A61K 45/06; C07K 14/4703; C12N 2310/14; A61K 31/713; G01N 33/6863; A61P 31/14; C12N 15/113

(57) ABSTRACT

The present invention relates to methods of modulating an immune response and/or cytokine production in a subject, the method comprises administering to the subject a compound which modifies C6orf106 protein activity. The present invention also relates to compounds for modifying C6orf106 protein activity in a subject, as well as to screening methods for identifying such compounds.

### FIELD OF THE INVENTION

The present invention relates to methods of modulating an immune response and/or cytokine production in a subject, the method comprising administering to the subject a compound which modifies C6orf106 protein activity. The present invention also relates to compounds for modifying C6orf106 protein activity in a subject, as well as to screening methods for identifying such compounds.

### BACKGROUND OF THE INVENTION

There is a requirement to identify modulators of the immune response for developing new methods of treating and/or preventing immune related disease. In particular, there is a requirement for new methods of increasing the immune response to infection, such as viral, bacterial, protozoan or fungal infection. Emerging infectious diseases are particularly problematic as there are often no treatment or prevention methods available for such infections. For example Zoonotic viruses emerging from wildlife and domesticated animals pose a serious threat to human and animal health and are recognised as the most likely source of the next pandemic. Containment of emerging infectious disease outbreaks is often difficult due to their unpredictability and the absence of effective control measures, such as antiviral therapeutics for humans. Thus, there is a requirement for modulators of the immune system which increase an immune response to provide protection against an infection.

Additionally there is also a requirement for new modulators of the immune response for increasing the immune response when a subject is experiencing immunodeficiency, for example in acquired immune deficiency syndrome (AIDS).

Further, there are requirements for modulators of the immune response which can decrease an overactive or inappropriate immune response. This is particularly important in conditions of chronic inflammation such as autoimmune disease. Such modulators are also of interest for the treatment of inflammation caused by injury e.g. injury caused by trauma. Such modulators can provide relief from inflammation.

Thus, there is a need to identify new targets, methods and compounds for modulating an immune response and/or cytokine production which can be used in methods for treating diseases such as infections, immunodeficiency, autoimmune disease and cancer.

### SUMMARY OF THE INVENTION

The present inventors have identified that modifying C6orf106 protein activity modulates an immune response and/or cytokine production.

Thus, in one aspect the present invention provides a method of modulating an immune response and/or cytokine production in a subject, the method comprising administering to the subject a compound which modifies C6orf106 protein activity.

The present inventors have also found that the C6orf106 protein binds IRF. Thus, in an embodiment, the compound modifies formation of a complex comprising C6orf106 and IRF3.

In an embodiment, the compound increases C6orf106 protein activity, and the immune response and/or cytokine production is reduced. In an embodiment, increased C6orf106 protein activity reduces IRF3-dependent cytokine transcription. In an embodiment, increasing C6orf106 protein activity reduces NF-κB activity. Examples of such compounds include, but are not limited to, a polynucleotide, a polypeptide or a small molecule.

In an embodiment, the polynucleotide encodes a polypeptide which comprises an amino acid sequence which is at least 50% identical to any one or more of SEQ ID NO's 1 to 11 or a biologically active fragment thereof. In an embodiment, the polynucleotide is operably linked to a promoter which directs expression of the polynucleotide in the subject. In an embodiment, the polynucleotide is an expression construct.

In an embodiment, the polynucleotide is administered in an expression vector. Any suitable expression vector can be used, examples of which include, but are not limited to, a eukaryotic, prokaryotic or viral vector. In an embodiment, the vector is a viral vector. In an embodiment, the viral vector is a retrovirus, lentivirus, an adenovirus, a herpes virus, a poxvirus, an adeno-associated viral vector or a vector derived therefrom.

In an embodiment, the polypeptide comprises an amino acid sequence which is at least 50% identical to any one or more of SEQ ID NO's 1 to 11 or a biologically active fragment thereof. In an embodiment, the biologically active fragment lacks a functional UBA-like domain. In an embodiment, the UBA-like domain comprises the amino acid sequence set forth in SEQ ID NO: 12. In an embodiment, the UBA-like domain comprises the amino acid sequence SEQ ID NO:13. In an embodiment, the biologically active fragment lacks a functional FW domain. In an embodiment, the FW domain comprises the amino acid set forth in SEQ ID NO: 57. In an embodiment, the FW domain comprises the amino acid sequence set forth in SEQ ID NO:58. In an embodiment, the FW domain comprises the amino acid sequence set forth in SEQ NO:60. In an embodiment, the biologically active fragment lacks a functional disordered region. In an embodiment, the disordered region comprises an amino acid sequence set forth in SEQ ID NO:61. In an embodiment, the disordered region comprises an amino acid sequence set forth in SEQ ID NO:62.

In an embodiment, the compound reduces C6orf106 protein activity, and the immune response and/or cytokine production is increased. In an embodiment, the compound reduces formation of a complex comprising C6orf106 and IRF3. In an embodiment, reducing C6orf106 protein activity increases IRF3-dependent cytokine transcription. In an embodiment, reducing C6orf106 protein activity increases NF-κB activity.

In an embodiment, the compound modifies the translocation of C6orf106 in a cell.

Examples of such compounds that increase or decrease C6orf106 protein activity include, but are not limited to, a polynucleotide, a polypeptide or a small molecule.

In an embodiment, the polynucleotide reduces expression of the C6orf106 gene. Examples of polynucleotides which can be used to reduce C6orf106 gene expression include, but are not limited to those selected from: an antisense polynucleotide, a sense polynucleotide, a polynucleotide which encodes a polypeptide which binds C6orf106, a double stranded RNA (dsRNA) molecule or a processed RNA molecule derived therefrom. In an embodiment, the dsRNA molecule is a siRNA, miRNA, shRNA or an aptamer.

In an embodiment, the polynucleotide is expressed from a transgene administered to the subject. In an embodiment, the transgene, is in a nucleic acid construct. In an embodiment, the transgene is in an expression vector.

In an embodiment, the polynucleotide binds to C6orf106 and reduces C6orf106 protein activity. In an embodiment, the polynucleotide is an RNA aptamer, a DNA aptamer, or an XNA aptamer. In an embodiment, the aptamer binds to the C6orf106 gene and reduces expression of the C6orf106 gene. In an embodiment, the aptamer binds to the C6orf106 protein and reduces C6orf106 protein activity.

In an embodiment, the compound binds to C6orf106 and reduces C6orf106 protein activity. In an embodiment, the compound is an antibody or antigenic binding fragment. In an embodiment, the antibody is a monoclonal antibody, humanized antibody, single chain antibody, diabody, triabody, or tetrabody.

In an embodiment, the compound is a programmable nuclease targeted to introduce a genetic modification into the C6orf106 gene or regulatory region thereof.

In an embodiment, the immune response is an IFN response. In an embodiment, the immune response is a type I IFN response.

In an embodiment, the cytokine is one, more or all of IFN-α, IFN-β and TNF-α. In an embodiment, the cytokine is IFN-α. In an embodiment, the cytokine is IFN-β. In an embodiment, the cytokine is TNF-α. In an embodiment, increasing C6orf106 protein activity inhibits transcription of one, more or all of IFN-α, IFN-β and TNF-α. In an embodiment, transcriptional regulation of IFN-α, IFN-β and TNF-α is independent of IRF3 and NFκB translation. In an embodiment, increasing C6orf106 protein activity inhibits IFN-α transcription. In an embodiment, increasing C6orf106 protein activity inhibits IFN-β transcription. In an embodiment, increasing C6orf106 protein activity inhibits TNF-α transcription. In an embodiment increasing C6orf106 protein activity does not modulate downstream IFN and/or TNF-α signalling, for example ISG15 or IκBα activity. In an embodiment, reducing C6orf106 protein activity increases IFN-α transcription. In an embodiment, reducing C6orf106 protein activity increases IFN-β transcription. In an embodiment, reducing C6orf106 protein activity increases TNF-α transcription.

In an embodiment, the immune response is selected from: an anti-viral immune response, an autoimmune response and an inflammatory response.

In an embodiment, the immune response is an anti-viral immune response and the immune response and/or cytokine production is increased.

In an embodiment, the immune response is an inflammatory response and the immune response and/or cytokine production is reduced.

In an embodiment, the subject has one or more of the following conditions: an infection, an immunodeficiency, an autoimmune disease, an inflammatory condition or cancer.

In an embodiment, the infection is a virus infection. In an embodiment, the virus in a negative-strand RNA virus.

In an embodiment, the virus is form the order: Mononegavirales, Herpesvirales or Nidovirales.

In an embodiment, the virus is selected from a: Orthomyxoviridae, Retroviridae, Herpesviridae, Paramyxoviridae, Rhabdoviridae, Filoviridae, Bornaviriade and Coronaviridae.

In an embodiment, the virus is from the order Mononegavirales. In an embodiment the Mononegavirales is selected from: Paramyxoviridae, Rhabdoviridae, Filoviridae and Bornaviriade.

In an embodiment, the subject is also administered with at least one antigen which stimulates an immune response. In an embodiment, the at least one antigen is a plant antigen (such as a pollen), a viral antigen, a bacterial antigen, a fungal antigen, a protozon antigen, or a tumor antigen. In an embodiment, the subject is administered with an antigen which stimulates an immune response to a virus.

In an embodiment, a compound which decreases C6orf106 activity is administered with at least one antigen or vaccine composition to increase the immune response to the at least one antigen or vaccine composition.

In an embodiment, a compound which decreases C6orf106 activity is administered with at least one cancer antigen to increase the immune response to the at least one cancer antigen.

In an embodiment, the autoimmune disease is selected from: Ulcerative colitis, Crohn's disease, Irritable bowel syndrome, Rheumatoid arthritis, Polyarthritis, Multiple sclerosis, Uveitis, asthma, Type 1 diabetes, Type 2 diabetes, Lupus or Chronic obstructive pulmonary disease.

In an embodiment, the subject is also administered with an antigen which stimulates an immune response to the cancer.

In an aspect, the present invention provides a method of treating and/or preventing an infection or cancer in a subject, the method comprising administering to the subject a compound which reduces C6orf106 protein activity.

In a further aspect, the present invention provides a method of treating and/or preventing autoimmune disease in a subject, the method comprising administering to the subject a compound which increases C6orf106 protein activity.

In an embodiment, C6orf106 comprises an amino acid sequence which is at least 50% identical to any one of SEQ ID NO's 1 to 11.

In an embodiment, the subject is an animal. In an embodiment, the subject is a mammal. In an embodiment, the subject is a human.

In an aspect, the present invention provides use of a compound which modifies C6orf106 protein activity in the manufacture of a medicament for modulating an immune response and/or cytokine production in a subject.

In another aspect, the present invention provides use of a compound that reduces C6orf106 protein activity in the manufacture of a medicament for treating an infection, immunodeficiency or cancer in a subject.

In another aspect, the present invention provides use of a compound that increases C6orf106 protein activity in the manufacture of a medicament for treating autoimmune disease in a subject.

In another aspect, the present invention provides a compound which modifies C6orf106 protein activity for use in modulating an immune response and/or cytokine production in a subject.

In another aspect, the present invention provides a compound which reduces C6orf106 protein activity for use in treatment of a virus infection or cancer.

In another aspect, the present invention provides a compound which increases C6orf106 protein activity for use in treatment of an autoimmune disease.

In another aspect, the present invention provides a compound which modifies formation of a complex comprising C6orf106 and IRF3 for use in modulating an immune response and/or cytokine production in a subject.

In another aspect, the present invention provides a method of identifying a compound which modifies C6orf106 protein activity, the method comprising:

i) contacting a cell with a candidate compound, and

ii) determining whether the compound increases or reduces C6orf106 protein activity in the cell.

In another aspect, the present invention provides a method of identifying a compound which modifies C6orf106 protein activity, the method comprising:

i) contacting a cell with a candidate compound, and

ii) determining whether the compound increases or reduces IRF3-dependent cytokine transcription in the cell.

In another aspect, the present invention provides a method of identifying compound which reduces C6orf106 protein activity, the method comprising:

i) contacting a cell with a candidate compound, and

ii) determining whether the compound reduces formation of a complex comprising C6orf106 and IRF3 in the cell.

In an embodiment, the method further comprises testing the compound for its ability to modulate virus infection. In an embodiment, virus infection is assessed in vitro, in ovo on in vivo. In an embodiment, virus infection is assessed in vitro. In an embodiment, virus infection is assessed in vitro in HeLa cells.

In an embodiment, the method comprises determining the level of C6orf106 mRNA in the cell. In an embodiment, the cell is a mammalian or avian cell. In an embodiment, the cell is a human cell such as a HeLa cell. In an embodiment, the mRNA level is determined by PCR such as qRT-PCR.

In an embodiment, the method comprises determining the level of C6orf106 protein the cell. In an embodiment, the level of C6orf106 protein is determined by an immunoassay. Exemplary immunoassay formats include immunoblot, Western blot, dot blot, enzyme linked immunosorbent assay (ELISA), radioimmunoassay (RIA) and enzyme immunoassay.

In another aspect, the present invention provides a method of identifying a compound that binds C6orf106, the method comprising:

i) contacting a polypeptide which comprises an amino acid sequence which is at least 50% identical to any one of SEQ ID NO's 1 to 11 or a biologically active fragment thereof, with a candidate compound, and

ii) determining whether the compound binds the polypeptide.

In an embodiment, the candidate compound is an antibody or fragment thereof, an aptamer or a small molecule.

In another aspect, the present invention provides a method of identifying a compound which modifies C6orf106 protein activity in silico, the method comprising:

i) generating a three dimensional structural model of a polypeptide comprising an amino acid sequence which is at least 50% identical to any one of SEQ ID NO's 1 to 11 or a biologically active fragment thereof, and

ii) designing or screening for a compound which potentially binds the structure, and/or

iii) designing or screening for a compound that modifies formation of a complex comprising C6orf106 and IRF3.

In an embodiment, the method further comprises testing the compound designed or screened for in ii) for its ability to bind C6orf106 and modulate C6orf106 protein activity. In an embodiment, the ability to bind and modulate C6orf106 protein activity is determined by an immunoassay. Exemplary immunoassay formats include immunoblot, Western blot, dot blot, enzyme linked immunosorbent assay (ELISA), radioimmunoassay (RIA) and enzyme immunoassay.

In an embodiment, the method further comprises testing the compound designed or screened for in ii) for its ability to modulate virus infection. In an embodiment, virus infection is assessed in vitro, in ovo on in vivo. In an embodiment, virus infection is assessed in vitro. In an embodiment, virus infection is assessed in vitro in HeLa cells.

In an embodiment, modulating C6orf106 protein activity increases C6orf106 protein activity.

In an embodiment, modulating C6orf106 protein activity reduces C6orf106 protein activity.

In an aspect, the present invention provides an isolated and/or recombinant mutant of a naturally occurring C6orf106 polypeptide which has a modified activity compared to the naturally occurring molecule.

In an embodiment, the present invention provides an isolated and/or recombinant polypeptide which comprises an amino acid sequence which is at least 50% identical to any one of SEQ ID NO's 1 to 11 but lacks a functional UBA-like domain. In an embodiment, the UBA-like domain lacks about 76 N-terminal amino acids amino of any one of SEQ ID NO's 1 to 11. In an embodiment, the UBA-like domain comprises the amino acid sequence set forth in SEQ ID NO:12. In an embodiment, the UBA-like domain comprises the amino acid sequence set forth in SEQ ID NO:13. In an embodiment, the isolated and/or recombinant polypeptide which comprises an amino acid sequence which is at least 50% identical to any one of SEQ ID NO's 1 to 11 but lacks a functional FW domain. In an embodiment, the FW domain comprises the amino acid sequence set forth in SEQ ID NO: 57. In an embodiment, the FW domain comprises the amino acid sequence set forth in SEQ ID NO:58. In an embodiment, the FW domain comprises the amino acid sequence set forth in SEQ ID NO:60. In an embodiment, the isolated and/or recombinant polypeptide which comprises an amino acid sequence which is at least 50% identical to any one of SEQ ID NO's 1 to 11 but lacks a functional disordered region. In an embodiment, the disordered region comprises the amino acid sequence set forth in SEQ ID NO:61. In an embodiment, the disordered region comprises the amino acid sequence set forth in SEQ ID NO:62.

In another aspect, the present invention provides an isolated and/or exogenous polynucleotide encoding the polypeptide of the invention.

In another aspect, the present invention provides a composition comprising an isolated and/or exogenous polynucleotide encoding the polypeptide of the invention. In an embodiment, the composition further comprises one or more excipients. In an embodiment, the composition further comprises at least one antigen which stimulates an immune response.

The steps, features, integers, compositions and/or compounds disclosed herein or indicated in the specification of this application individually or collectively, and any combinations of two or more of said steps or features.

Any embodiment herein shall be taken to apply mutatis mutandis to any other embodiment unless specifically stated otherwise. For instance, as the skilled person would understand examples of compounds outlined above for methods of the invention equally apply to the uses of the invention.

The present invention is not to be limited in scope by the specific embodiments described herein, which are intended for the purpose of exemplification only. Functionally-equivalent products, compositions and methods are clearly within the scope of the invention, as described herein.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

The invention is hereinafter described by way of the following non-limiting Examples and with reference to the accompanying figures.

### BRIEF DESCRIPTION OF THE ACCOMPANYING DRAWINGS

FIG. 1. C6orf106 knockdown reduces virus production. (A) and (B) HeLa cells were transfected with a siRNA pool targeting C6orf106 or non-specific RNA (siNT1) for 48 hours. Cells were then infected with Hendra (HeV), Mumps (MuV), Influenza A (H5N1), Vesicular Stomatitis Virus (VSV) or West Nile Virus (WNV) at an multiplicity of infection (moi) 5 for 24 hours. At this time, virus production was assayed by TCID₅₀ on Vero cells. Error bars indicate +1 standard deviation of at least 3 replicates, and significance (shown by asterisk) was determined by 1-way ANOVA with a Dunn's multiple comparison test. (C) Cell numbers 72 h post-transfection with siRNAs. Data is normalised to siNEG values. (D) Relative cell metabolic activity, measured by Alamar blue assay, in cells transfected with siRNAs as described above.

FIG. 2. C6orf106 is highly conserved across vertebrate species with two putative functional domains and a disordered C-terminal region. The C6orf106 protein sequence was sourced from NCBI (Accession number Q9H6K1.2) and aligned to various vertebrate C6orf106 sequences predicted from Ensembl (www.ensembl.org) using ClustalW. Asterisks indicate identical residues and the boxes indicate the position of the putative UBA-like and FW domains respectively. The disordered C-terminus (as predicted by Globpot (http://globplot.embl.de/cgiDict.py) and PSI prediction software (http://bioinf.cs.ucl.ac.uk/psipred).

FIG. 3. C6orf106 knockdown enhances cytokine transcription in response to poly(I:C). HeLa cells were transfected with a siRNA pool targeting C6orf106 or non-specific RNAs (siNT1/siNT2) for 48 hours, then stimulated with poly(I:C) for 6 hours. (A) Gene knockdown was assayed by qRT-PCR and western blotting with C6orf106-specific primers/antibodies respectively. (B) Relative expression of interferons and pro-inflammatory cytokines was measured using qRT-PCR with gene-specific primers, compared to GAPDH internal controls.

FIG. 4. C6orf106 overexpression inhibits interferon α/β transcription and secretion in response to poly(I:C). HeLa cells were transfected with Flag-tagged C6orf106 (C6-Flag) or eGFP or vector alone (pCAGGs) for 24 hours, then stimulated with poly(I:C) for 6 hours. (A) Relative expression of interferons and pro-inflammatory cytokines was measured using qRT-PCR with gene-specific primers, compared to GAPDH internal controls. (B) Inteferon β secretion into cell culture supernatants was detected by ELISA.

FIG. 5. Endogenous cytokine and C6orf106 RNA levels increase with time of poly(I:C) stimulation. (A) Mock-transfected HeLa cells were stimulated with poly(I:C) for 6 hours, then RNA extracted and cytokine levels determined by qPCR analysis. (B) C6orf106 and IFN-b mRNA levels in HeLa cells after stimulation with poly(I:C) (5 μg/mL) for indicated timepoints. Error bars indicate −/+1 standard deviation of 3 independent experiments, and asterisks indicate significant differences (compared to 0 hour) as determined by 1-way ANOVA with Dunn's multiple comparison test. (C) HeLa cells treated as in FIG. 4 were transfected with IFN-β- or NF-κB-firefly and Renilla-luciferase vectors for 24 h. Celt lysates were assayed for luciferase activity following stimulation with poly(I:C) (5 μg/mL, 6 h), relative levels were normalised to the transfection control Renilla-luciferase.

FIG. 6. Deletion of the UBA-like domain enhances the inhibitory effect of C6orf106 on cytokine transcription. (A) C6orf106 deletion mutants were generated using the codon-optimised pCAGGs-C6orf106 vector as a template. (B) HeLa cells were transfected with Flag-tagged C6orf106 deletion mutants or vector alone (pCAGGs) for 24 hours, then stimulated with poly(I:C) for 6 hours. Relative expression of interferons and pro-inflammatory cytokines was measured using qRT-PCR with gene-specific primers, compared to GAPDH internal controls. (C) HeLa cells were reverse-transfected with equal amounts of Flag-IRF3 and C6 deletion mutant expression vectors for 24 hours, then stimulated with 5 μg/mL poly(I:C) for 6 hours. Cells were then lysed and subjected to direct co-immunoprecipitation with an anti-IRF3 antibody, IP samples (and input controls) were then probed with anti-Flag and anti-IRF3 antibodies by western blotting.

FIG. 7. C6orf106 overexpression does not impair nuclear translocation of transcription factors in response to poly(I:C). HeLa cells were transfected with Flag-tagged C6orf106 (C6-Flag) or eGFP or vector alone (pCAGGs) for 24 hours, then stimulated with poly(I:C) for 6 hours. (A) Cells were fixed and labelled with anti-C6 and anti-p65 antibodies and counterstained with the nuclear stain Dapi or (B) anti-C6 and anti-IRF3 antibodies and counterstained with the nuclear stain Dapi and viewed on a Leica SP5 confocal microscope.

FIG. 8. C6orf106 does not impair activation or nuclear translocation of transcription factors in response to poly(I:C). HeLa cells were treated as described in FIG. 7. (A) The Fn/c ratios for treatment groups of cells transfected with Flag-tagged C6orf106 (C6-Flag) or eGFP with and without poly(I:C) treatment. Error bars indicate −/+1 standard deviation of a typical experiment from duplicate experiments; asterisks indicate significant differences as determined by 1-way ANOVA with Dunn's multiple comparison test. (B) HeLa cells as treated in FIG. 7 were lysed and separated into cytosolic and nuclear fractions. Fractions were probed for the transcription factors IRF3 and p65, as well as C6 and the loading control GAPDH.

FIG. 9. Endogenous C6orf106 RNA levels increase with time of poly(I:C) stimulation. (A) HeLa cells were stimulated with 10 μg/mL poly(I:C) for 4, 6 and 9 hours, and endogenous C6orf106 RNA levels assessed by qRT-PCR (bars on the right) compared to mock at same time point (bars on the left). (B) IFN/TNF-α induced signalling is not significantly impaired by C6orf106 overexpression.

FIG. 10. C6orf106 forms a complex with IRF3. (A) C6orf106 forms a complex with IRF3 and this binding is enhanced by poly(I:C). HeLa cells treated as in FIG. 4 were lysed and subjected to indirect immunoprecipitation with an anti-IRF3 antibody. IP samples (and input controls) were then probed with anti-C6 and anti-IRF3 antibodies by western blotting. (B) To show that C6orf106 forms a complex with IRF3 HEK293T transfected with IRF3 alone, or in combination with C6orf106 were lysed and subjected to indirect immunoprecipitation with an anti-IRF3 antibody. IP samples and input controls were probed with anti-FLAG antibody for western blotting. An IgG isotype was used as a negative control for the immunoprecipitation experiment.

FIG. 11. C6-FLAG deletion mutants have different subcellular localisations and effects on IRF3 nuclear trafficking. Hela cells were transfected with FLAG-tagged C6orf106 deletion mutants or vector alone (pCAGGs) for 24 hours, then stimulated with 5 μg/mL poly(I:C) for 6 hours. Cells were fixed and stained with anti-C6/FLAG (green) and IRF3 (red) antibodies and viewed on a Leica SP5 confocal microscope. (A) images collected are representative of two independent experiments, bar indicates 10 μm. Images were then analysed using ImageJ software to calculate Fn/c ratios for IRF3 (B) or the deletion mutants (C). Error bars indicate −/+1 standard deviation of a typical experiment from duplicate experiments; asterisks indicate significant differences as determined by 1-way ANOVA with Dunn's multiple comparison test. Percentages above each column represent the proportion of cells with an Fn/c ratio above the threshold of 0.5 (D) HeLa cells as in (A) were lysed and separated into cytosolic and nuclear fractions. Fractions were probed for the transcription factors IRF3 and p65, as well as C6 and/or FLAG.

### KEY TO THE SEQUENCE LISTING

SEQ ID NO:1—H. sapiens C6orf106 protein sequence;
SEQ ID NO:2—G. gorilla C6orf106 protein sequence;
SEQ ID NO:3—C. sabaeus C6orf106 protein sequence;
SEQ ID NO:4—M. musculus C6orf106 protein sequence;
SEQ ID NO:5—M. lucifugus C6orf106 protein sequence;
SEQ ID NO:6—C. familiaris C6orf106 protein sequence;
SEQ ID NO:7—G. gallus C6orf106 protein sequence;
SEQ ID NO:8—A. carolinensis C6orf106 protein sequence;
SEQ ID NO:9—X. tropicalis C6orf106 protein sequence;
SEQ ID NO:10—D. rerio C6orf106 protein sequence;
SEQ ID NO:11—C. intestinalis C6orf106 protein sequence;
SEQ ID NO:12—UBA-like domain of the H. sapiens C6orf106 protein sequence;
SEQ ID NO:13—consensus sequence of the C6orf106 UBA-like domain;
SEQ ID NO:14—primer SacI-START-C6 For;
SEQ ID NO:15—primer XhoI-C6-FLAG Rev;
SEQ ID NO:16—primer XhoI-C6(1-276)-FLAG Rev;
SEQ ID NO:17—primer XhoI-C6(1-193)-FLAG Rev;
SEQ ID NO:18—primer XhoI-C6(1-76)-FLAG Rev;
SEQ ID NO:19—primer SacI-START-C6-UBA For;
SEQ ID NO:20—primer IFN-α For;
SEQ ID NO:21—primer IFN-α For;
SEQ ID NO:22—primer IFN-β For;
SEQ ID NO:23—primer IFN-β Rev;
SEQ ID NO:24—primer IL-6 For;
SEQ ID NO:25—primer IL-6 Rev;
SEQ ID NO:26—primer TNF-α For;
SEQ ID NO:27—primer TNF-α Rev;
SEQ ID NO:28—primer ISG15 For;
SEQ ID NO:29—primer ISG15 Rev;
SEQ ID NO:30—primer IκBα For;
SEQ ID NO:31—primer IκBα Rev;
SEQ ID NO:32—primer huC6orf106 For;
SEQ ID NO:33—primer huC6orf106 Rev;
SEQ ID NO:34—primer GAPDH For;
SEQ ID NO:35—primer GAPDH Rev;
SEQ ID NO:36—siGENOME human C6orf106 SMARTpool target sequence D-016330-02-;
SEQ ID NO:37—siGENOME human C6orf106 SMARTpool target sequence D-016330-03-;
SEQ ID NO:38—siGENOME human C6orf106 SMARTpool target sequence D-016330-04-;
SEQ ID NO:39—siGENOME human C6orf106 SMARTpool target sequence D-016330-17-;
SEQ ID NO:40—C6orf106-FLAG protein sequence;
SEQ ID NO:41—C6orf106 (1-276)-FLAG protein sequence;
SEQ ID NO:42—C6orf106 (1-193)-FLAG protein sequence/C6orf106 (delta dis)-FLAG protein sequence;
SEQ ID NO:43—C6orf106 (1-76)-FLAG protein sequence;
SEQ ID NO:44—C6orf106 (dUBA)-FLAG protein sequence;
SEQ ID NO:45—C6orf106 (dFW)-FLAG protein sequence;
SEQ ID NO:46—H. sapiens C6orf106 nucleotide sequence;
SEQ ID NO:47—G. gorilla C6orf106 nucleotide sequence;
SEQ ID NO:48—C. sabaeus C6orf106 nucleotide sequence;
SEQ ID NO:49—M. musculus C6orf106 nucleotide sequence;
SEQ ID NO:50—M. lucifugus C6orf106 nucleotide sequence;
SEQ ID NO:51—C. familiaris C6orf106 nucleotide sequence;
SEQ ID NO:52—G. gallus C6orf106 nucleotide sequence;
SEQ ID NO:53—A. carolinensis C6orf106 nucleotide sequence;
SEQ ID NO:54—X. tropicalis C6orf106 nucleotide sequence;
SEQ ID NO:55—D. rerio C6orf106 nucleotide sequence;
SEQ ID NO:56—C. intestinalis C6orf106 nucleotide sequence;
SEQ ID NO:57—FW domain of the H. sapiens C6orf106 protein sequence;
SEQ ID NO:58—consensus sequence of the C6orf106 FW domain;
SEQ ID NO:59—C6(deltaFW)-FLAG protein sequence;
SEQ ID NO:60—FW domain of the H. sapiens C6orf106 protein sequence;
SEQ ID NO:61—disordered region of the H. sapiens C6orf106 protein sequence;
SEQ ID NO:62—disordered region of the H. sapiens C6orf106 protein sequence.

### DETAILED DESCRIPTION OF THE INVENTION

### General Techniques and Selected Definitions

Unless specifically defined otherwise, all technical and scientific terms used herein shall be taken to have the same meaning as commonly understood by one of ordinary skill in the art (e.g., molecular biology, cell culture, immunology, immunohistochemistry, protein chemistry, and biochemistry).

Unless otherwise indicated, the cell culture and immunological techniques utilized in the present invention are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

The term “and/or”, e.g., “X and/or Y” shall be understood to mean either “X and Y” or “X or Y” and shall be taken to provide explicit support for both meanings or for either meaning.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

A “transgene” as referred to herein has the normal meaning in the art of biotechnology and includes a genetic sequence which has been produced or altered by recombinant DNA or RNA technology and can be used to modify C6orf106 protein activity. In one example, the transgene encodes a siRNA, miRNA, shRNA which is targeted to silence expression of the C6orf106 gene. In one example the transgene encodes an aptamer which recognizes and binds to the C6orf106 gene or protein. In one example, the transgene comprises the C6orf106 gene or a fragment thereof. In one example the transgene encodes a binding protein, such as an antibody, which recognizes and binds to the C6orf106 gene and/or protein. In one example, the transgene encodes a programmable nuclease and optionally one or more targeting sequences directed at recognition of the C6orf106 gene. The transgene can be introduced into cells of the subject by any method one of skill in the art recognizes. A transgene includes genetic sequences that are introduced into a chromosome as well as those that are extrachromosomal. The transgene will typically comprise an open reading frame encoding a polynucleotide as described herein operably linked to a suitable promoter for expressing the polynucleotide.

The term “small molecule” as used herein, refers to a chemical compound or molecule having a molecular weight below 2000 Daltons, preferably below 1500 Daltons, more preferably below 1000 Daltons, still more preferably below 750 daltons, yet more preferably below 500 Daltons, in an embodiment, the small molecule is not a polypeptide.

As used herein, the term “subject” can be any animal. In one example, the animal is a vertebrate. For example, the animal can be a mammal, avian, arthropod, chordate, amphibian or reptile. Exemplary subjects include but are not limited to human, primate, livestock (e.g. sheep, cow, chicken, horse, donkey, pig), companion animals (e.g. dogs, cats), laboratory test animals (e.g. mice, rabbits, rats, guinea pigs, hamsters), captive wild animal (e.g. fox, deer). In one example, the mammal is a human.

As used e the terms “treating” or “treatment” include administering a therapeutically effective amount of a compound described herein sufficient to reduce or eliminate at least one symptom of a specified disease or condition.

As used herein, the terms “prevent” or “preventing” include administering a therapeutically effective amount of a compound described herein sufficient to stop or hinder the development of at least one symptom of a specified disease or condition. In an embodiment, the compound reduces infection of cells by a virus.

As used herein, the term “complex comprising C6orf106 and IRF3” refers to a complex comprising at least C6orf106 and IRF3. The complex may additionally comprise one or more one or more proteins, one or more RNA molecules, one or more DNA molecules or a combination thereof.

As used herein, the term “IRF3 comprising complex” includes a complex of one or more proteins, one or more RNA molecules, one or more DNA molecules or a combination thereof which includes or binds to the IRF3 protein.

As used herein, the term “negative-strand RNA virus” or “antisense-strand RNA virus” includes a virus whose genetic information consists of a single strand of RNA that is the negative or antisense strand and does not encode RNA.

### Immune Response

As used herein, the term “immune response” has its ordinary meaning in the art, and includes both humoral and cellular immunity. An immune response can manifest as one or more of, the development of anti-antigen antibodies, expansion of antigen-specific T cells, increase in tumor infiltrating-lymphocytes (TILs), development of an anti-tumor or anti-tumor antigen delayed-type hypersensitivity (DTH) response, clearance of the pathogen, suppression of pathogen and/or tumor growth and/or spread, tumor reduction, reduction or elimination of metastases, increased time to relapse, increased time of pathogen or tumor free survival, and increased time of survival. An immune response may be mediated by one or more of, B-cell activation, T-cell activation, natural killer cell activation, activation of antigen presenting cells (e.g., B cells, DCs, monocytes and/or macrophages), cytokine production, chemokine production, specific cell surface marker expression, in particular, expression of co-stimulatory molecules. The immune response may be characterized by a humoral, cellular, Th1 or Th2 response, or combinations thereof. In an embodiment, the immune response is an innate immune response.

In an embodiment, the immune response is an IFN response, in an embodiment, the immune response is a type I IFN response. In an embodiment, the immune response is a type II IFN response. In an embodiment, the immune response is a type III IFN response. In an embodiment, the immune response comprises expression of one or more or all of IFN-α, IFNβ and TNF-α. In an embodiment, the immune response can be measured by measuring the level of one or more of IFN-α, and TNF-α.

As used herein, the term “modulating an immune response” refers to increasing or reducing an immune response to a stimulus. Modulation of an immune response can be measured by any method known to a person skilled in the art and can involve, for example, measuring the levels of one or more cytokines. Examples of suitable methods for measuring cytokine production are provided in the Examples section.

As used herein, the term “modulating cytokine production” refers to increasing or reducing the production of cytokine in the subject. In an embodiment, modulating cytokine production refers to increasing or reducing secretion of cytokines by a cell of the subject. In an embodiment, production of the cytokine is mediated by IRF3 activity. In an embodiment, the cytokine is in the IFN pathway. In an embodiment, the cytokine is one or more or all of IFN-α, IFNβ and TNF-α. Modulating cytokine production can be measured using any method known to a person skilled in the art including but not limited to those described herein in the Examples section. The level of cytokine production can be measured, for example, in a patient sample such as a blood, serum or plasma sample.

As used herein, the term “IFN response” refers to an immune response which involves the IFN pathway. In an embodiment, the immune response is a type I IFN immune response. In an embodiment, the immune response is a type II IFN immune response. In an embodiment, the immune response is a type III IFN immune response. In an embodiment, the IFN response involves one or more or all of IFN-α, IFNβ and TNF-α.

Compounds which Modify C6orf106 Protein Activity

As used herein, the term “modifies C6orf106 protein activity” refers to modifying the ability of C6orf106 to modulate an immune response and/or cytokine production. In an embodiment, reducing the level of C6orf106 mRNA or C6orf106 protein modifies the C6orf106 protein activity by reducing the activity. In an embodiment, a compound which binds to C6orf106 and modulates the activity of C6orf106, for example reducing or inhibiting C6orf106 protein activity is considered to modify C6orf106 protein activity. In an embodiment, a compound which modulates the activity of C6orf106 modulates its ability to bind to IRF3. In an embodiment, a compound which modulates the activity of C6orf106 modulates formation of a complex comprising C6orf106 and IRF3. In an embodiment, a compound which modulates the activity of C6orf106 modulates its ability to form a complex with IRF3. In an embodiment, increasing the level of C6orf106 mRNA or C6orf106 protein or a biologically active fragment thereof modifies the C6orf106 protein activity by increasing the activity. In an embodiment, modulating an agonist, inhibitor, receptor, or protein involved in the cellular trafficking of C6orf106 can modify C6orf106 protein activity. In an embodiment, a compound which modifies C6orf106 protein activity does not significantly alter the metabolic activity of the cells treated. In an embodiment, the metabolic activity of the cells is assessed with the Alamar blue assay.

As used herein, the term “reduce” or “reduces” or “reduced” or “reducing” refers to abolishing, decreasing or having a lower level of gene expression, protein activity, an immune response or cytokine production compared to that present in the state before a compound was administered. In an embodiment, gene expression, protein activity, an immune response and/or cytokine production is reduced by at least 5%, or at least 10%, or at least 15%, or at least 20%, or at least 25%, or at least 30%, or at least 35%, or at least 40%, or at least 45%, or at least 50%, or at least 60%, or at least 70%, or at least 80%, or at least 90%, or at least 100% compared to that present in the state before the compound was administered.

As used herein, the term “increase” or “increases” or “increased” or “increasing” refers to having a higher or greater level of gene expression, protein activity, an immune response or cytokine production compared to that present in the state before a compound was administered. In an embodiment, gene expression, protein activity, an immune response and/or cytokine production is increased by at least 5%, or at least 10%, or at least 15%, or at least 20%, or at least 25%, or at least 30%, or at least 35%, or at least 40%, or at least 45%, or at least 50%, or at least 60%, or at least 70%, or at least 80%, or at least 90%, or at least 100% compared to that present in the state before the compound was administered.

### Polynucleotides

The terms “polynucleotide”, and “nucleic acid” are used interchangeably. A polynucleotide is a polymer of nucleotide monomers. A polynucleotide suitable for use in the method of the invention may be of any length and can comprise deoxyribonucleotides or ribonucleotides, or analogs thereof, or a mixture thereof. A polynucleotide suitable for use in the method of the invention may be of genomic, cDNA, semisynthetic, or synthetic origin, double-stranded or single-stranded and by virtue of its origin or manipulation: (1) is not associated with all or a portion of a polynucleotide with which it is associated in nature, (2) is linked to a polynucleotide other than that to which it is linked in nature (for example, a promoter), or (3) does not occur in nature. The following are non-limiting examples of polynucleotides: coding or non-coding regions of a gene or gene fragment, loci (locus) defined from linkage analysis, exons, introns, messenger RNA (mRNA), transfer RNA (tRNA), ribosomal RNA (rRNA), ribozymes, cDNA, recombinant polynucleotides, branched polynucleotides, plasmids, vectors, isolated DNA, isolated RNA, chimeric DNA, nucleic acid probes, and primers. A polynucleotide may comprise modified nucleotides such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization such as by conjugation with a labelling component.

By “isolated polynucleotide” it is meant a polynucleotide which has generally been separated from the polynucleotide sequences with which it is associated or linked in its native state. Preferably, the isolated polynucleotide is at least 60% free, more preferably at least 75% free, and more preferably at least 90% free from the polynucleotide sequences with which it is naturally associated or linked.

By “exogenous polynucleotide” it is meant a polynucleotide present in a cell free expression system or a cell that does not naturally comprise the polynucleotide or a polynucleotide expressed in an altered amount or expressed at an altered rate (e.g., in the case of mRNA) compared to its native state. In an embodiment, the polynucleotide is introduced into a cell that does not naturally comprise the polynucleotide. Typically an exogenous DNA is used as a template for transcription of mRNA which is then translated into a continuous sequence of amino acid residues coding for a polypeptide within the transformed cell. In another embodiment, the polynucleotide, is endogenous to the cell and its expression is altered by recombinant means, for example, an exogenous control sequence is introduced upstream of an endogenous gene of interest to enable the transformed cell to express the polypeptide encoded by the gene.

An exogenous polynucleotide suitable for use in the invention includes polynucleotides which have not been separated from other components of the cell-based or cell-free expression system, in which it is present, and polynucleotides produced in said cell-based or cell-free systems which are subsequently purified away from at least some other components.

A polynucleotide of, or useful for, the present invention may selectively hybridise,under stringent conditions, to a polynucleotide defined herein.

Polynucleotides of the invention may possess, when compared to reference polynucleotides, one or more mutations which are deletions, insertions, or substitutions of nucleotide residues. Polynucleotides which have mutations relative to a reference sequence can be either naturally occurring (that is to say, isolated from a natural source) or synthetic (for example, by performing site-directed mutagenesis or DNA shuffling on the nucleic acid). Polynucleotides of the invention may possess a truncation. The truncation may be an N-terminal or C-terminal truncation.

In an embodiment, a polynucleotide which modifies C6orf106 protein activity may be modified or optimized to enhance its activity. Modifications or analogs of nucleotides can be introduced to improve the properties of the polynucleotides. Improved properties include increased nuclease resistance and/or increased ability to permeate cell membranes. Accordingly, the terms “polynucleotide” includes synthetically modified bases such as, but not limited to, inosine, xanthine, hypoxanthine, 2-aminoadenine, 6-methyl-, 2-propyl- and other alkyl- adenines, 5-halo uracil, 5-halo cytosine, 6-aza cytosine and 6-aza thymine, pseudo uracil, 4-thiuracil, 8-halo adenine, 8-aminoadenine, 8-thiol adenine, 8-thiolalkyl adenines, 8-hydroxyl adenine and other 8-substituted adenines, 8-halo guanines, 8-amino guanine, 8-thiol guanine, 8-thioalkyl guanines, 8-hydroxyl guanine and other substituted guanines, other aza and deaza adenines, other aza and deaza guanines, 5-trifluoromethyl uracil and 5-trifluoro cytosine.

### Double Stranded RNA or DNA

In an embodiment, the polynucleotide is a dsRNA. In one embodiment, expression of the C6orf106 gene is reduced by a transgene which encodes a dsRNA molecule for RNAi.

The terms “RNA interference”, “RNAi” or “gene silencing” refer generally to a process in which a dsRNA molecule reduces the expression of a nucleic acid sequence with which the double-stranded RNA molecule shares substantial or total homology. However, it has been shown that RNA interference can be achieved using non-RNA double stranded molecules (see, for example, US 20070004667).

The present invention includes a polynucleotide comprising and/or encoding double-stranded regions for RNA interference for use in the invention. The polynucleotides are typically RNA but may comprise chemically-modified nucleotides and non-nucleotides.

The double-stranded regions should be at least 19 contiguous nucleotides, for example about 19 to 23 nucleotides, or may be longer, for example 30 or 50 nucleotides, or 100 nucleotides or more. The full-length sequence corresponding to the entire gene transcript may be used. Preferably, they are about 19 to about 23 nucleotides in length.

The degree of identity of a double-stranded region of a nucleic acid molecule to the targeted transcript should be at least 90% and more preferably 95-100%. The nucleic acid molecule may of course comprise unrelated sequences which may function to stabilize the molecule.

The term “short interfering RNA” or “siRNA” as used herein refers to a polynucleotide which comprises ribonucleotides capable of inhibiting or down regulating gene expression, for example by mediating RNAi in a sequence-specific manner, wherein the double stranded portion is less than 50 nucleotides in length, preferably about 19 to about 23 nucleotides in length. For example the siRNA can be a nucleic acid molecule comprising self-complementary sense and antisense regions, wherein the antisense region comprises nucleotide sequence that is complementary to nucleotide sequence in a target nucleic acid molecule or a portion thereof and the sense region having nucleotide sequence corresponding to the target nucleic acid sequence or a portion thereof. The siRNA can be assembled from two separate oligonucleotides, where one strand is the sense strand and the other is the antisense strand, wherein the antisense and sense strands are self-complementary.

As used herein, the term siRNA is meant to be equivalent to other terms used to describe polynucleotides that are capable of mediating sequence specific RNAi, for example micro-RNA (miRNA), short hairpin RNA (shRNA), short interfering oligonucleotide, short interfering nucleic acid (siNA), short interfering modified oligonucleotide, chemically-modified siRNA, post-transcriptional gene silencing RNA (ptgsRNA), and others. In addition, as used herein, the term RNAi is meant to be equivalent to other terms used to describe sequence specific RNA interference, such as post transcriptional gene silencing, translational inhibition, or epigenetics. For example, siRNA molecules can be used to epigenetically silence genes at both the post-transcriptional level or the pre-transcriptional level. In a non-limiting example, epigenetic regulation of gene expression by siRNA molecules can result from siRNA mediated modification of chromatin structure to alter gene expression. In one embodiment, the siRNA used in the method of the present invention is one or more of SEQ ID NO: 36 to SEQ ID NO: 39.

By “shRNA” or “short-hairpin RNA” is meant an RNA molecule where less than about 50 nucleotides, preferably about 19 to about 23 nucleotides, is base paired with a complementary sequence located on the same RNA molecule, and where said sequence and complementary sequence are separated by an unpaired region of at least about 4 to about 15 nucleotides which forms a single-stranded loop above the stern structure created by the two regions of base complementarity. An Example of a sequence of a single-stranded loop includes: 5′ UUCAAGAGA 3′.

Included shRNAs are dual or bi-finger and multi-finger hairpin dsRNAs, in which the RNA molecule comprises two or more of such stem-loop structures separated by single-stranded spacer regions.

As used herein “aptamer” is a single stranded nucleic acid that has a three dimensional conformation capable of recognizing and binding the C6orf106 gene or the C6orf106 protein resulting in modified C6orf106 gene or protein activity. In an embodiment, the aptamer is DNA or RNA. In an embodiment, the aptamer is a mixture of DNA and RNA and/or can contain one or more modified bases or base analogues, this is referred to herein as XNA.

Once designed, the polynucleotides comprising a double-stranded region can be generated by any method known in the art, for example, by in vitro transcription, recombinantly, or by synthetic means.

### Polypeptides

The terms “polypeptide” and “protein” are generally used interchangeably. In some embodiments, the polypeptide binds to and/or reduces the expression or activity of C6orf106. In some embodiments, the polypeptide increases the expression or activity of C6orf106. In some embodiments, the polypeptide binds to an agonist, inhibitor, receptor of C6orf106 or a cellular component involved in the trafficking and/or cellular localization of C6orf106. In one embodiment, the polypeptide is C6orf106 or a biologically active fragment thereof. In one embodiment, the polypeptide modulates the formation of a complex comprising C6orf106 and IRF3.

Before the present invention, C6orf106 had been poorly-characterized (Mungall et al., 2003; Zhang et al., 2015; Jiang et al., 2015). As used herein, the term “C6orf106” refers to “uncharacterized protein C6orf106”, “Chromosome 6 Open Reading Frame 106”, “DJ391O22.4” or “FP852”. In an embodiment, C6orf106 is H. sapiens C6orf106 corresponding to Gene ID 64771 or Ensembl identifier ENSG00000196821. In an embodiment, C6orf106 is G. gorilla C6orf106 corresponding to Gene ID 101134934 or Ensembl identifier ENSGGOG00000000388. In an embodiment, C6orf106 is C. sabaeus C6orf106 corresponding to Gene ID 103221631 or Ensembl identifier ENSCSAG00000009818. In an embodiment, C6orf106 is M. musculus C6orf106 corresponding to Gene ID 224647 or Ensembl identifier ENSMUSG00000056692. In an embodiment, C6orf106 is M. lucifugus C6orf106 corresponding to Ensembl identifier ENSMLUG00000002482. In an embodiment, C6orf106 is C. familiaris C6orf106 corresponding to Gene ID 100685164 or to Ensembl identifier ENSCAFG00000001229. In an embodiment, C6orf106 is G. gallus C6orf106 corresponding to Gene ID 419902 or to Ensembl identifier ENSGALG00000002778. In an embodiment, C6orf106 is A. carolinensis C6orf106 corresponding to Gene ID 100552720 or to Ensembl identifier ENSACAG00000015742. In an embodiment, C6orf106 is X. tropicalis C6orf106 corresponding to Ensembl identifier ENSXETG00000031607. In an embodiment, C6orf106 is D. rerio C6orf106 corresponding to Gene ID 541415 or to Ensembl identifier ENSDARG00000078075, In an embodiment, C6orf106 is C. intestinalis C6orf106 corresponding to Ensembl identifier ENSCING00000020052. In an embodiment, C6orf106 is encoded by any one or more of SEQ ID NO:46 to 56 or a sequence that is at least 50%, or at least 60%, or at least 65%, or at least 70%, or at least 75%, or at least 80%, or at least 85%, or at least 90%, or at least 95%, or 100% identical to SEQ ID NO: 46 to 56 or a biologically active fragment thereof. In an embodiment, C6orf106 has an amino acid sequences that is at least 45%, or at least 50%, or at least 54%, or at least 60%, or at least 65%, or at least 70%, or at least 75%, or at least 80%, or at least 85%, or at least 90%, or at least 91%, or at least 92%, or at least 93%, or at least 94%, or at least 95 or at least 96%, or at least 97%, or at least 98%, or at least 99% or 100% identical to SEQ ID NO's 1 to 11 or a biological active fragment thereof. The polypeptide may have the same activity as, or an enhanced activity relative to the reference polypeptide.

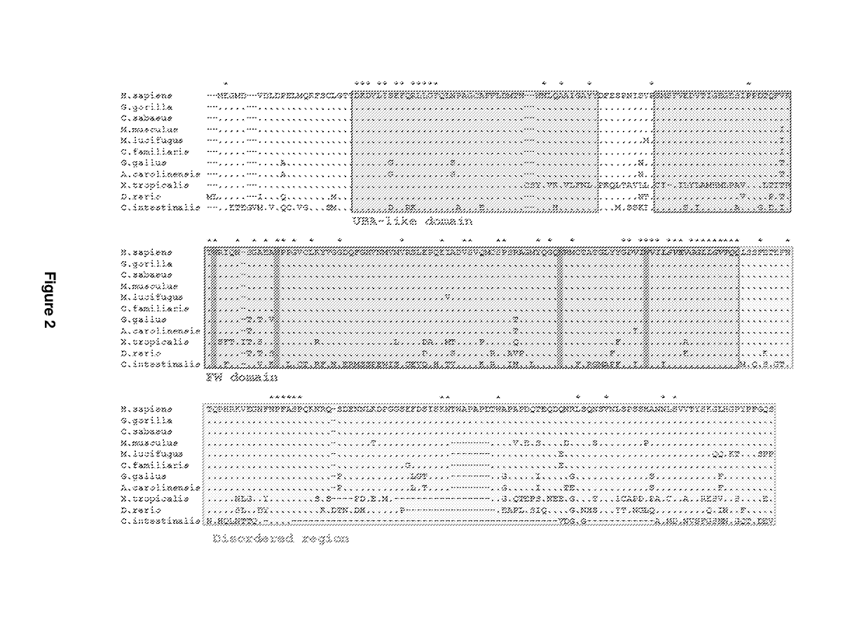

As used herein the term “biologically active fragment” refers to a fragment of C6orf106 that has the ability to modulate an immune response and/or cytokine production. Biologically active fragments as used herein exclude the full-length polypeptide. Biologically active fragments can be any size portion as long as they maintain the defined activity. In an embodiment, the biologically active fragment binds to IRF3. In an embodiment, the biologically active fragment binds to an IRF3 comprising complex. In an embodiment, the biologically active fragment lacks one or more putative domains selected from: a functional UBA-like domain, a functional disordered region or a functional FW domain (domains and regions are indicated in FIG. 2). In an embodiment, the biologically active fragment lacks a functional UBA-like domain. In an embodiment, the biologically active fragment lack a functional disordered region. In an embodiment, the biologically active fragment lacks a functional FW domain. In an embodiment, the biologically actice fragment lacks a functional UBA-like domain and a functional disordered region. Preferably, the biologically active fragment maintains at least 10%, at least 50%, at least 75%, or at least 90%, of the activity of the full length protein or has enhanced activity relative to the full length protein. Preferably, the biologically active fragment has enhanced activity relative to the full length protein.

As used herein, the term “ubiquitin-associated-like domain” “UBA-like domain” refers to an N-terminal domain of C6orf106 as shown in FIG. 2. In an embodiment, the UBA-like domain refers to amino acids 23 to 63 of SEQ ID NO's 1 to 8: or amino acid residues of 25 to 65 of SEQ ID NO's 10 or 11; or amino acid residues 23 to 66 of SEQ ID NO: 9; or comprises the amino acid sequence set forth in SEQ ID NO:12, or SEQ ID NO:13 or is a sequence which is at least 50% identical to any of the aforementioned sequences. As used herein, the phrase, lacking a “functional UBA-like domain” refers to C6orf106 which is lacking the UBA-like domain, lacking a fragment of the UBA-like domain, or comprises one or more mutations in the UBA-like domain such as a substitution, insertion or deletion which disrupts the function of the UBA-like domain. In an embodiment, C6orf106 lacking the UBA-domain lacks about 76 N-terminal amino acids of any one of SEQ ID NO's 1 to 11.

As used herein, the term “disordered region” refers to the C-terminal region of C6orf106 comprising the residues as indicated in FIG. 2. In an embodiment, the disordered region comprises the amino acid sequence set forth in SEQ ID NO:61 or is a sequence which is at least 50% identical thereto. In an embodiment, the disordered region comprises the amino acid sequence set forth in SEQ ID NO:62 or is a sequence which is at least 50% identical thereto. In an embodiment, the disordered region refers to amino acids 241 to 298 of SEQ ID NO's 1 to 3: or amino acid residues 241 to 291 of SEQ ID NO's 4 to 8; or amino acid residues of 244 to 285 of SEQ ID NO 10; or amino acid residues 244 to 281 of SEQ ID NO: 11; or amino acid residues :244 to 281 of SEQ ID NO: 9 or is a sequence which is at least 50% identical to any of the aforementioned sequences.

As used herein, the term “FW domain” or “Nbr-1-like domain” refers to a C6orf106 domain comprising the residues as indicated in FIG. 2. In an embodiment, the FW domain refers to amino acids 98 to 190 of SEQ ID NO's 1 to 8: or amino acid residues of 100 to 192 of SEQ ID NO's 10 or 11; or amino acid residues 100 to 194 of SEQ ID NO: 9; comprises the amino acid sequence set forth in SEQ ID NO:57, or SEQ ID NO:58, or SEQ ID NO”60 or is a sequence which is at least 50% identical to any of the aforementioned sequences.

A polypeptide suitable for use in a method of the invention may be defined by the extent of identity (% identity) of its amino acid sequence to a reference amino acid sequence, or by having a greater % identity to one reference amino acid sequence than to another. The % identity of a polypeptide to a reference amino acid sequence is typically determined by GAP analysis (Needleman and Wunsch, 1970 GCG program) with parameters of a gap creation penalty=5, and a gap extension penalty=0.3. The query sequence is at least 100 amino acids in length and the GAP analysis aligns the two sequences over a region of at least 100 amino acids. Even more preferably, the query sequence is at least 250 amino acids in length and the GAP analysis aligns the two sequences over a region of at least 250 amino acids. Even more preferably, the query sequence is at least 290 amino acids in length and the GAP analysis aligns the two sequences over a region of at least 290 amino acids. Even more preferably, the query sequence is at least 300 amino acids in length and the GAP analysis aligns the two sequences over a region of at least 300 amino acids. Even more preferably, the GAP analysis aligns two sequences over their entire length.

Amino acid sequence mutants of the polypeptides of SEQ ID NO's 1 to 11 can he prepared by introducing appropriate nucleotide changes into a nucleic acid defined herein, or by in vitro synthesis of the desired polypeptide. Such mutants include for example, deletions, insertions, or substitutions of residues within the amino acid sequence. A combination of deletions, insertions and substitutions can be made to arrive at the final construct, provided that the final polypeptide product possesses the defined activity.

Mutant (altered) polypeptides can be prepared using any technique known in the art, for example, using directed evolution or rational design strategies (see below). Products derived from mutated/altered DNA can readily be screened using techniques described herein to determine if they possess the ability to modulate C6orf106 protein activity.

In designing amino acid sequence mutants, the location of the mutation site and the nature of the mutation will depend on characteristic(s) to be modified. The sites for mutation can be modified individually or in series for example, by (1) substituting first with conservative amino acid choices and then with more radical selections depending upon the results achieved, (2) deleting the target residue, or (3) inserting other residues adjacent to the located site.

Amino acid sequence deletions generally range from about 1 to 15 residues, more preferably about 1 to 10 residues and typically about 1 to 5 contiguous residues.

Substitution mutants have at least one amino acid residue in the polypeptide removed and a different residue inserted in its place. The sites of greatest interest for substitutional mutagenesis include sites identified as the active site(s) for example substrate or co-factor binding sites. Other sites of interest are those in which particular residues obtained from various strains or species are identical. These positions may be important for biological activity. These sites, especially those falling within a sequence of at least three other identically conserved sites, are preferably substituted in a relatively conservative manner. Such conservative substitutions are shown in Table 1 under the heading of “exemplary substitutions”.

In a preferred embodiment a mutant/variant polypeptide has only, or not more than, one or two or three or four conservative amino acid changes when compared to a reference polypeptide. Details of conservative amino acid changes are provided in Table 1. As the skilled person would be aware, such minor changes can reasonably be predicted not to alter the activity of the polypeptide when expressed in a cell.

TABLE 1
Exemplary substitutions

| Original | Exemplary |
|---|---|
| Residue | Substitutions |
| Ala (A) | val; leu; ile; gly |
| Arg (R) | lys |
| Asn (N) | gln; his |
| Asp (D) | glu |
| Cys (C) | ser |
| Gln (Q) | asn; his |
| Glu (E) | asp |
| Gly (G) | pro, ala |
| His (H) | asn; gln |
| Ile (I) | leu; val; ala |
| Leu (L) | ile; val; met; ala; phe |
| Lys (K) | arg |
| Met (M) | leu; phe |
| Phe (F) | leu; val; ala |
| Pro (P) | gly |
| Ser (S) | thr |
| Thr (T) | ser |
| Trp (W) | tyr |
| Tyr (Y) | trp; phe |
| Val (V) | ile; leu; met; phe, ala |

Furthermore, if desired, unnatural amino acids or synthetic amino acid analogues can be introduced as a substitution or addition into the polypeptides. Such amino acids include, but are not limited to, the D-isomers of the common amino acids, 2,4-diaminobutyric acid, α-amino isobutyric acid, 4-aminobutyric acid, 2-aminobutyric acid, 6-amino hexanoic acid, 2-amino isobutyric acid, 3-amino propionic acid, ornithine, norleucine, norvaline, hydroxyproline, sarcosine, citrulline, homocitrulline, cysteic acid, t-butylglycine, t-butylalanine, phenylglycine, cyclohexylalanine, β-alanine, fluoro-amino acids, designer amino acids such as β-methyl amino acids, Cα-methyl amino acids, Nα-methyl amino acids, and amino acid analogues in general.

Also included within the scope are polypeptides which are differentially modified during or after synthesis, for example, by biotinylation, benzylation, glycosylation, acetylation, phosphorylation, amidation, derivatization by known protecting/blocking groups, proteolytic cleavage, linkage to an antibody molecule or other cellular ligand, etc. These modifications may serve to increase the stability and/or bioactivity of the polypeptides.

In an embodiment, a polypeptide which modifies C6orf1 06 protein activity may be modified or optimized to enhance its activity and/or stability. This may occur by diversification or selection. In directed evolution, random mutagenesis is applied to a protein, and a selection regime is used to pick out variants that have the desired qualities, for example, increased activity. Further, rounds of mutation and selection are then applied. A typical directed evolution strategy involves three steps:

1) Diversification: The polypeptide is mutated and/or recombined at random to create a large library of gene variants. Variant gene libraries can be constructed through error prone PCR (see, for example, Leung, 1989; Cadwell and Joyce, 1992), from pools of DNaseI digested fragments prepared from parental templates (Stemmer, 1994a; Stemmer, 1994b; Crameri et al., 1998; Coco et al., 2001) from degenerate oligonucleotides (Ness et al., 2002, Coco, 2002) or from mixtures of both, or even from undigested parental templates (Zhao et al., 1998; Eggert et al., 2005) and are usually assembled through PCR. Libraries can also be made from parental sequences recombined in vivo or in vitro by either homologous or non-homologous recombination (Ostermeier et al., 1999; Volkov et at., 1999; Sieber et al., 2001). Variant gene libraries can also be constructed by sub-cloning a gene of interest into a suitable vector, transforming the vector into a “mutator” strain such as the E. coli XL-1 red (Stratagene) and propagating the transformed bacteria for a suitable number of generations. Variant gene libraries can also be constructed by subjecting the polypeptide of interest to DNA shuffling (i.e., in vitro homologous recombination of pools of selected mutant genes by random fragmentation and reassembly) as broadly described by Harayama (1998).

2) Selection: The library is tested for the presence of mutants (variants) possessing the desired property using a screen or selection. Screens enable the identification and isolation of high-performing mutants by hand, while selections automatically eliminate all nonfunctional mutants. A screen may involve screening for the presence of known conserved amino acid motifs. Alternatively, or in addition, a screen may involve expressing the mutated polynucleotide in a cell or transgenic non-human organism or part thereof and assaying the level of, for example, the ability to modulate C6orf106 protein activity, for example, quantifying the level of resultant product in the cell or transgenic non-human organism or part thereof or extracted from the cell or transgenic non-human organism or part thereof, and determining the level of product relative to a corresponding cell or transgenic non-human organism or part thereof lacking the mutated polynucleotide and optionally, expressing the parent (unmutated) polynucleotide. Alternatively, the screen may involve feeding the cell or transgenic non-human organism or part thereof labeled substrate and determining the level of substrate or product in the cell or transgenic non-human organism or part thereof, or extracted from the cell or transgenic non-human organism or part thereof relative to a corresponding cell or transgenic non-human organism or part thereof lacking the mutated polynucleotide and optionally, expressing the parent (unmutated) polynucleotide.

3) Amplification: The variants identified in the selection or screen are replicated many fold, enabling researchers to sequence their DNA in order to understand what mutations have occurred.

Together, these three steps are termed a “round” of directed evolution. Most experiments will entail more than one round. In these experiments, the “winners” of the previous round are diversified in the next round to create a new library. At the end of the experiment, all evolved protein or polynucleotide mutants are characterized using biochemical methods.

### Binding Agents

In an embodiment, the polypeptide is a binding agent in an embodiment, the binding agent is an antibody or a fragment thereof. In some embodiments, the antibody binds to and reduces the expression or activity of C6orf106. In some embodiments, the antibody binds to a polypeptide to which C6orf106 interacts such as a receptor, inhibitor or agonist thereof. In an embodiment, the antibody is an antibody modified to penetrate or be taken up (passively or actively) by a cell.

The term “antibody” as used herein includes polyclonal antibodies, monoclonal antibodies, bispecific antibodies, fusion diabodies, triabodies, heteroconjugate antibodies, chimeric antibodies including intact molecules as well as fragments thereof, and other antibody-like molecules. Antibodies include modifications in a variety of forms including, for example, but not limited to, domain antibodies including either the VH or VL domain, a dimer of the heavy chain variable region (VHH, as described for a camelid), a dimer of the light chain variable region (VLL), Fv fragments containing only the light (VL) and heavy chain (VH) variable regions which may be joined directly or through a linker, or Fd fragments containing the heavy chain variable region and the CH1 domain. The skilled person will understand that the antibody can be any antibody that binds to C6orf106, such as those available at Santa Cruz Biotechnolgy (e.g. sc-398490).

A scFv consisting of the variable regions of the heavy and light chains linked together to form a single-chain antibody (Bird et al., 1988; Huston et al., 1988) and oligomers of scFvs such as diabodies and triabodies are also encompassed by the term “antibody”. Also encompassed are fragments of antibodies such as Fab, (Fab′)2 and FabFc2 fragments which contain the variable regions and parts of the constant regions. Complementarity determining region (CDR)-grafted antibody fragments and oligomers of antibody fragments are also encompassed. The heavy and light chain components of an Fv may be derived from the same antibody or different antibodies thereby producing a chimeric Fv region. The antibody may be of animal (for example mouse, rabbit or rat) or may be chimeric (Morrison et al., 1984). The antibody may be produced by any method known in the art.

Using the guidelines provided herein and those methods well known to those skilled in the art which are described in the references cited above and in such publications as Harlow & Lane, Antibodies: a Laboratory Manual, Cold Spring Harbor Laboratory, (1988) the antibodies for use in the methods of the present invention can be readily made.

The antibodies may be Fv regions comprising a variable light (VL) and a variable heavy (VH) chain in which the light and heavy chains may be joined directly or through a linker. As used herein a linker refers to a molecule that is covalently linked to the light and heavy chain and provides enough spacing and flexibility between the two chains such that they are able to achieve a conformation in which they are capable of specifically binding the epitope to which they are directed. Protein linkers are particularly preferred as they may be expressed as an intrinsic component of the Ig portion of the fusion polypeptide.

In one embodiment, the antibodies have the capacity for intracellular transmission. Antibodies which have the capacity for intracellular transmission include antibodies such as camelids and llama antibodies, shark antibodies (IgNARs), scFv antibodies, intrabodies or nanobodies, for example, scFv intrabodies and VHH intrabodies. Such antigen binding agents can be made as described by Harmsen and De Haard (2007), Tibary et al. (2007) and Muyldermans et al. (2001). Yeast SPLINT antibody libraries are available for testing for intrabodies which are able to disrupt protein-protein interactions (see for example, Visintin et al. (2008) for methods for their production). Such agents may comprise a cell-penetrating peptide sequence or nuclear-localizing peptide sequence such as those disclosed in Constantini et al. (2008). Also useful for in vivo delivery are Vectocell or Diato peptide vectors such as those disclosed in De Coupade et al. (2005) and Meyer-Losic et al. (2006).

In addition, the antibodies may be fused to a cell penetrating agent, for example a cell-penetrating peptide. Cell penetrating peptides include Tat peptides, Penetratin, short amphipathic peptides such as those from the Pep-and MPG-families, oligoarginine and oligolysine. In one example, the cell penetrating peptide is also conjugated to a lipid (C6-C18 fatty acid) domain to improve intracellular delivery (Koppelhus et at., 2008). Examples of cell penetrating peptides can be found in Howl et al. (2007) and Deshayes et al. (2008). Thus, the invention also provides the use of antibodies fused via a covalent bond (e.g. a peptide bond), at optionally the N-terminus or the C-terminus, to a cell-penetrating peptide sequence.

Polypeptides suitable for use in a method of the present invention, including C6orf106 or a biologically active fragment thereof, can be produced in a variety of ways, including production and recovery of natural polypeptides, production and recovery of recombinant polypeptides, and synthetic synthesis of the polypeptides. In an embodiment, an isolated polypeptide is produced by culturing a cell capable of expressing the polypeptide under conditions effective to produce the polypeptide, and recovering the polypeptide. Effective culture conditions include, but are not limited to, effective media, bioreactor, temperature, pH and oxygen conditions that permit polypeptide production. An effective medium refers to any medium in which a cell is cultured to produce a polypeptide. Such medium typically comprises an aqueous medium having assimilable carbon, nitrogen and phosphate sources, and appropriate salts, minerals, metals and other nutrients, such as vitamins. Cells can be cultured in conventional fermentation bioreactors, shake flasks, test tubes, microtiter dishes, and petri plates. Culturing can be carried out at a temperature, pH and oxygen content appropriate for a host cell. Such culturing conditions are within the expertise of one of ordinary skill in the art.

### Programmable Nucleases

In an embodiment, the polypeptide is a programmable nuclease which modifies C6orf106 protein activity by modifying the C6orf106 gene. As used herein, the term “programmable nuclease” relates to nucleases that are “targeted” (“programed”) to recognize and edit a pre-determined genomic location. In an embodiment, the protein is a programmable nuclease “targeted” or “programmed” to introduce a genetic modification into the C6orf106 gene or regulatory region thereof. In an embodiment, the genetic modification is a deletion, substitution or in C6orf106 or a regulatory region thereof.

In an embodiment, the programmable nuclease may be programmed to recognize a genomic location by a combination of DNA-binding zinc-finger protein (ZFP) domains. ZFPs recognize a specific 3-bp in a DNA sequence, a combination of ZFPs can be used to recognize a specific genomic location. In an embodiment, the programmable nuclease may be programmed to recognize a genomic location by transcription activator-like effectors (TALEs) DNA binding domains. In an alternate embodiment, the programmable nuclease may be programmed to recognize a genomic location by one or more RNA sequences. In an alternate embodiment, the programmable nuclease may be programmed by one or more DNA sequences. In an alternate embodiment, the programmable nuclease may be programmed by one or more hybrid DNA/RNA sequences. In an alternate embodiment, the programmable nuclease may be programmed by one or more of an RNA sequence, a DNA sequences and a hybrid DNA/RNA sequence.

Programmable nucleases that can be used in accordance with the present disclosure include, but are not limited to, RNA-guided engineered nuclease (RGEN) derived from the bacterial clustered regularly interspaced short palindromic repeat (CRISPR)-cas (CRISPR-associated) system, zinc-finger nuclease (ZFN), transcription activator-like nuclease (TALEN), and argonautes.

In an embodiment, the nuclease is a RNA-guided engineered nuclease (RGEN). In an embodiment, the RGEN is from an archaeal genome or is a recombinant version thereof. In an embodiment, the RGEN is from a bacterial genome or is a recombinant version thereof. In an embodiment, the RGEN is from a Type I (CRISPR)-cas (CRISPR-associated) system. In an embodiment, the RGEN is from a Type II (CRISPR)-cas (CRISPR-associated) system. In an embodiment, the RGEN is from a Type III (CRISPR)-cas (CRISPR-associated) system. In an embodiment, the nuclease is a class I RGEN. In an embodiment, the nuclease is a class II RGEN. In an embodiment, the RGEN is a multi-component enzyme. In an embodiment, the RGEN is a single component enzyme. In an embodiment, the RGEN is CAS3. In an embodiment, the RGEN is CAS10. In an embodiment, the RGEN is CAS9. In an embodiment, the RGEN is Cpf1 (Zetsche et al., 2015). In an embodiment, the RGEN is targeted by a single RNA or DNA. In an embodiment, the RGEN is targeted by more than one RNA and/or DNA. In an embodiment, the programmable nuclease may be a DNA programmed argonaute (WO 14/189628). In an embodiment, the CAS9 is from Streptococcus pyogenes.

### Small Molecules

In an embodiment, the compound which modifies C6orf106 protein actvity is a small molecule. In an embodiment, the small molecule increases C6orf106 protein activity. In an embodiment, the small molecule reduces C6orf106 protein activity. In an embodiment, the small molecule binds to the C6orf106 gene and inhibits its expression. In an embodiment, the small molecule binds C6orf106 protein reducing the ability of C6orf106 to perform its normal function, reducing C6orf106 protein activity. In an embodiment, the small molecule binds the C6orf106 protein enhancing its activity thereby increasing C6orf106 protein activity. In an embodiment, the small molecule binds a polypeptide that interacts with C6orf106, such as an agonist, receptor or protein involved in the cellular trafficking or localization of C6orf106 reducing or increasing C6orf106 protein activity.

in an embodiment, the compound that is administered may be a precursor compound which is inactive or comparatively poorly active, but which following administration is converted (e.g. metabolised) to a compound reduces the expression of an antiviral gene and/or protein activity in the population of cells when compared to isogenic cells lacking the compound. In those embodiments, the compound that is administered may be referred to as a prodrug. Alternatively or in addition, the compounds that are administered may be metabolized to produce active metabolites which have activity in reducing the expression of an antiviral gene and/or protein activity in the population of cells when compared to isogenic cells lacking the compound. The use of such active metabolites is also within the scope of the present disclosure.

Depending on the substituents present in the compound, the compound may optionally be present in the form of a salt. Salts of compounds which are suitable for use in the invention are those in which a counter ion is pharmaceutically acceptable. Suitable salts include those formed with organic or inorganic acids or bases. In particular, suitable salts formed with acids include those formed with mineral acids, strong organic carboxylic acids, such as alkane carboxylic acids of 1 to 4 carbon atoms which are unsubstituted or substituted, for example, by halogen, such as saturated or unsaturated dicarboxylic acids, such as hydroxycarboxylic acids, such as amino acids, or with organic sulfonic acids, such as (C1-4)-alkyl- or aryl-sulfonic acids which are substituted or unsubstituted, for example by halogen. Pharmaceutically acceptable acid addition salts include those formed from hydrochloric, hydrobromic, sulphuric, nitric, citric, tartaric, acetic, phosphoric, lactic, pyruvic, acetic, trifluoroacetic, succinic, perchloric, fumaric, maleic, glycolic, lactic, salicylic, oxaloacetic, methanesulfonic, ethanesulfonic, p-toluenesulfonic, formic, benzoic, malonic, naphthalene-2-sulfonic, benzenesulfonic, isethionic, ascorbic, malic, phthalic, aspartic, and glutamic acids, lysine and arginine. Pharmaceutically acceptable base salts include ammonium salts, alkali metal salts, for example those of potassium and sodium, alkaline earth metal salts, for example those of calcium and magnesium, and salts with organic bases, for example dicyclohexylamine, N-methyl-D-glucomine, morpholine, thiomorpholine, piperidien, pyrrolidine, a mono-, di- or tri-lower alkylamine, for example ethyl-, t-butyl-, diethyl-, diisopropyl-, triethyl-, tributyl- or dimethyl-propylamine, or a mono-, di- or trihydroxy lower alkylamine, for example mono-, di- or triethanolamine. Corresponding internal salts may also be formed.

Those skilled in the art of organic and/or medicinal chemistry will appreciate that many organic compounds can form complexes with solvents in which they are reacted or from which they are precipitated or crystallised. These complexes are known as “solvates”. For example, a complex with water is known as a “hydrate”. Solvates, such as hydrates, exist when the drug substance incorporates solvent, such as water, in the crystal lattice in either stoichiometric or non-stoichiometric amounts. Drug substances are routinely screened for the existence of solvates such as hydrates since these may be encountered at any stage. Accordingly it will be understood that the compounds useful for the present invention may be present in the form of solvates, such as hydrates. Solvated forms of the compounds which are suitable for use in the invention are those wherein the associated solvent is pharmaceutically acceptable. For example, a hydrate is an example of a pharmaceutically acceptable solvate.

The compounds useful for the present invention may be present in amorphous form or crystalline form. Many compounds exist in multiple polymorphic forms, and the use of the compounds in all such forms is encompassed by the present disclosure.

Small molecules useful for the present disclosure can be identified using standard procedures such as screening a library of candidate compounds for binding to C6orf106 protein, and then determining if any of the compounds which bind reduce C6orf106 protein activity. In an embodiment, screening for a compound of the invention comprises assessing whether the compound modulates viral infection in vitro, in ovo or in vivo. Small molecules useful for the present disclosure can also be identified using standard procedures of in silica screening, which can include screening of known library compounds, to identify candidates which bind to C6orf106 gene or protein and reduce C6orf106 protein activity.

### Delivery of Polynucleotides or Polypeptides

### Nucleic Acid Construct

In an embodiment, a polynucleotide is a nucleic acid construct. In an embodiment, the nucleic acid construct may comprise a transgene. As used herein, “nucleic acid construct” refers to any nucleic acid molecule that encodes, for example, a double-stranded RNA molecule (e.g. siRNA, miRNA, shRNA or an aptamer), a RNA, DNA or RNA/DNA hybrid sequences which “guides” or “targets” a programmable nuclease, or a polynucleotide which encodes a polypeptide in a vector. Typically, the nucleic acid construct will be double stranded DNA or double-stranded RNA, or a combination thereof. Furthermore, the nucleic acid construct will typically comprise a suitable promoter operably linked to an open reading frame encoding the polynucleotide. The nucleic acid construct may comprise, for example, a first open reading frame encoding a first single strand of the double-stranded RNA molecule, with the complementary (second) strand being encoded by a second open reading frame by a different, or preferably the same, nucleic acid construct. The nucleic acid construct may be a linear fragment or a circular molecule and it may or may not be capable of replication. The skilled person will understand that the nucleic acid construct of the invention may be included within a suitable expression vector. Transfection or transformation of the nucleic acid construct into a recipient cell allows the cell to express an RNA or DNA molecule encoded by the nucleic acid construct.

In another example, the nucleic acid construct may express multiple copies of the same, and/or one or more (e.g. 1, 2, 3, 4, 5, or more) including multiple different, RNA molecules comprising a double-stranded region, for example a short hairpin RNA. In an embodiment, the nucleic acid construct may express a sequence encoding one or more aptamers an aptamer or a sequence which can be processed to produce an aptamer. In one example, the nucleic acid construct, is a construct suitable for homologous recombination.

The nucleic acid construct also may contain additional genetic elements. The types of elements that may be included in the construct are not limited in any way and may be chosen by one with skill in the art. In some embodiments, the nucleic acid construct is inserted into a host cell as a transgene. In an embodiment, the host cell is an immune cell. In an embodiment, the immune cell is a leucocyte. In an embodiment the leucocyte is a lymphocyte or a phagocyte. In an embodiment, the lymphocyte is a T-cell or a B-cell. In such instances it may be desirable to include “stuffer” fragments in the construct which are designed to protect the sequences encoding the RNA molecule from the transgene insertion process and to reduce the risk of external transcription read through. Stuffer fragments may also be included in the construct to increase the distance between, e.g., a promoter and a coding sequence and/or terminator component. The stuffer fragment can be any length from 5-5000 or more nucleotides. There can be one or more stuffer fragments between promoters. In the case of multiple stuffer fragments, they can be the same or different lengths. The stuffer DNA fragments are preferably different sequences. Preferably, the stuffer sequences comprise a sequence identical to that found within a cell, or progeny thereof, in which they have been inserted. In a further embodiment, the nucleic acid construct comprises stuffer regions flanking the open reading frame(s) encoding the double stranded RNA(s).

Alternatively, the nucleic acid construct may include a transposable element, for example a transposon characterized by terminal inverted repeat sequences flanking the open reading frames encoding the double stranded RNA(s). Examples of suitable transposons include Tol2, mini-Tol, Sleeping Beauty, Mariner and Galluhop.

Other examples of an additional genetic element which may be included in the nucleic acid construct include a reporter gene, such as one or more genes for a fluorescent marker protein such as GFP or RFP; an easily assayed enzyme such as beta-galactosidase, luciferase, beta-glucuronidase, chloramphenical acetyl transferase or secreted embryonic alkaline phosphatase; or proteins for which immunoassays are readily available such as hormones or cytokines. Other genetic elements that may find use in embodiments include those coding for proteins which confer a selective growth advantage on cells such as adenosine deaminase, aminoglycodic phosphotransferase, dihydrofolate reductase, hygromycin-B-phosphotransferase or drug resistance.

In an embodiment, the additional genetic element may be one or more polynucleotides encoding an antigen which stimulates an immune response in the subject.

Where the nucleic acid construct is to be transfected into a cell, it is desirable that the promoter and any additional genetic elements consist of nucleotide sequences that naturally occur in the hosts genome. In an embodiment, the nucleic acid construct comprises a promoter. The skilled person will appreciate that a promoter such as a constitutive promoter or an inducible promoter can be used in the present invention. In an embodiment, the promoter is a Pol I, Pol II or Pol III promoter.

### Expression Vectors

In some instances it may be desirable to insert the polynucleotide or nucleic acid construct into an expression vector. In an embodiment, the expression vector may be transferred into a cell and the cell used to produce a polynucleotide and/or polypeptide suitable for use in methods of the present invention. In an embodiment, the expression vector may be transferred into a cell of a subject to allow the polynucleotide and/or polypeptide to be expressed in the subject.

Thus, for example, cells from a subject may be engineered with a polynucleotide, such as a DNA or RNA, to encode a polypeptide ex vivo. The engineered cells can then be provided to a subject to be treated with the polynucleotide or polypeptide. In this embodiment, cells may be engineered ex vivo, for example, by the use of a vector containing RNA encoding a polypeptide useful for the methods of the present invention can be used to transform cells. Such methods are well-known in the art and their use in the present invention will be apparent from the teachings herein.

Further, cells may be engineered in vivo for expression of a polypeptide in vivo by procedures known in the art. The expression construct may then be isolated. In one example a packaging cell is transduced with a plasmid vector containing RNA encoding a polypeptide useful for a method of the present invention, such that the packaging cell now produces infectious viral particles containing the gene of interest. These producer cells may be administered to a subject for engineering cells in vivo and expression of the polynucleotide and/or polypeptide in vivo. In an embodiment, the expression vector is administered directly to the subject. These and other methods for administering a polypeptide of the present invention should be apparent to those skilled in the art from the teachings of the present invention.

As used herein, an “expression vector” is a DNA or RNA vector that is capable of transforming a host cell and of effecting expression of one or more polynucleotides. Preferably, the expression vector is also capable of replicating within the host cell. Expression vectors can be either prokaryotic, eukaryotic or viral. Expression vectors include any vectors that function (i.e., direct gene expression) in host cells of the present invention, including in bacterial, fungal, endoparasite, arthropod, animal, plant and algal cells. The vector can be either RNA or DNA. The vector may be, e.g., a plasmid, virus, artificial chromosome, or a bacteriophage. Such vectors include chromosomal, episomal and virus-derived vectors, e.g., vectors derived from bacterial plasmids, bacteriophages, and viruses, vectors derived from combinations thereof, such as those derived from plasmid and bacteriophage genetic elements, cosmids and phagemids. In an embodiment, the vector is a viral vector. In an embodiment, the viral vector is a retrovirus, a lentivirus, an adenovirus, a herpes virus, a poxvirus or an adeno-associated viral vector.

Retroviruses from which the retroviral plasmid vectors may be derived include, but are not limited to, Moloney Murine Leukemia Virus, Spleen Necrosis Virus, Rous Sarcoma Virus, Harvey Sarcoma Virus, Avian Leukosis Virus, Gibbon Ape Leukemia Virus, Human Immunodeficiency Virus, Adenovirus, Myeloproliferative Sarcoma Virus, and Mammary Tumor Virus. In an embodiment, the retroviral plasmid vector is derived from Moloney Murine Leukemia Virus.

Such vectors will include one or more promoters for expressing the polypeptide. Suitable promoters which may be employed include, but are not limited to, the retroviral LTR; the SV40 promoter; and the human cytomegalovirus (CMV) promoter. Cellular promoters such as eukaryotic cellular promoters including, but not limited to, the histone, RNA polymerase III, and β-actin promoters, can also be used. Additional viral promoters which may be employed include, but are not limited to, adenovirus promoters, thymidine kinase (TK) promoters, and B19 parvovirus promoters. The selection of a suitable promoter will be apparent to those skilled in the art from the teachings contained herein.

The nucleic acid sequence encoding the polypeptide useful for a method of the present invention will be placed under the control of a suitable promoter. Suitable promoters which may be employed include, but are not limited to, adenoviral promoters, such as the adenoviral major late promoter; or heterologous promoters, such as the cytomegalovirus (CMV) promoter; the respiratory syncytial virus (RSV) promoter; inducible promoters, such as the MMT promoter, the metallothionein promoter; heat shock promoters; the albumin promoter; the ApoAI promoter; human globin promoters; viral thymidine kinase promoters, such as the Herpes Simplex thymidine kinase promoter; retroviral LTRs (including the modified retroviral LTRs herein above described); the β-actin promoter; and human growth hormone promoters. The promoter may also be the native promoter which controls the gene encoding the polypeptide.

The retroviral plasmid vector is employed to transduce packaging cell lines to form producer cell lines. Examples of packaging cells which may be transfected include, but are not limited to, the PE501, PA317, Y-2, Y-AM, PA12, T19-14X, VT-19-17-H2, YCRE, YCRIP, GP+E-86, GP+envAm12, and DAN cell lines as described in Miller (1990). The vector may be transduced into the packaging cells through any means known in the art. Such means include, but are not limited to, electroporation, the use of liposomes, and CaPO₄ precipitation. In one alternative, the retroviral plasmid vector may be encapsulated into a liposome, or coupled to a lipid, and then administered to a host.

The producer cell line will generate infectious retroviral vector particles, which include the nucleic acid sequence(s) encoding the polypeptides. Such retroviral vector particles may then be employed to transduce eukaryotic cells, either in vitro or in vivo. The transduced eukaryotic cells will express the nucleic acid sequence(s) encoding the polypeptide. Eukaryotic cells which may be transduced include, but are not limited to, mesenchemymal cells, chondrocytes, embryonic stem cells, embryonic carcinoma cells, as well as hematopoietic stem cells, hepatocytes, fibroblasts, myoblasts, keratinocytes, endothelial cells, and bronchial epithelial cells.

In an embodiment, the vector is suitable for gene therapy, such as an adeno-associated virus vector (Daya et al., 2008).

In an embodiment, the vector is a vector capable of expressing multiple functional RNAs or proteins or a combination thereof such as vectors described in Kabadi et al. (2014). Such vectors can be used for simultaneous administration of a programmable nuclease and one or more “guide” or “targeting” sequences. Such vectors can also be used for simultaneous expression of multiple double stranded RNAs.

In an embodiment, the vector is a vector which targets and preferably infects cancer cells such as Cavatak™ or Imlygic™ (talimogene laherparepvec).

Cells and Cell Culture

The skilled person would appreciate that a polypeptide or a fragment thereof as described herein can be produced in cell culture by the expression of a polynucleotide or expression vector as described herein. In one example the cells are prokaryotic or eukaryotic. In one example, the cells are of mammalian, avian, bacterial or Arthropoda origin. In one example, the cells are mammalian. In one example, the cells are from a continuous cell line. In one example, the cells are from a primary cell line. In one example, the cells are from an immortalized cell line. In one example, the cells are adherent cells. In one example, the cells are non-adherent cells (suspension cells). In one example, the cells are immune cells.

In one example, the mammalian cells are HEK, CHO or HeLa cells. In one example, the cells are HeLa cells. In one example, the cells are from a primary cell line derived from chicken embryonic fibroblasts (CEF). In one example, the cells are from the immortalized chick embryo cell line PBS-1. In one example, the cells are from the chicken fibroblast cell line DF-1. In one example, the cells are Madin-Darby canine kidney (MDCK) cells. In one example, the cells are MDCK 33016 cells. In one example, the cells are MDCK CCL34 cells. In one example, the cells African green monkey kidney-derived Vero cells. In one example, the cells are human retina derived PER.C6 cells. In one example, the cells are AGE1.CR cells. In one example, the cells are derived from the MRC-5 diploid cell line. In one example, the cells are human embryo kidney cells (HEK293). In an embodiment, the cell culture is bacterial cell culture. In an embodiment, the bacterial cells are Escherichia coli (E. coli). In one example, the cells are insect cells. In one example, the insect cells are derived from Trichoplusia. In one example, the cells can be cultured in the absence of serum. In one example, the cells are cultured in the presence of serum.

The population of cells of the present invention can be cultured in any cell culture medium that allows the expansion of the cells in vitro. Such mediums and processes will be known to the skilled person (see, for example, Genzel et al., 2009; Josefsberg et al., 2012; Wolf et al., 2011). Exemplary cell culture mediums for culturing the population of cells of the present invention include, but are not limited to: Iscove's medium, UltraCHO, CD Hybridoma serum free medium, episerf medium, MediV SF103 (serum free medium), Dulbecco's modified eagle medium (DMEM), Eagles Modified Eagle Medium (EMEM), Glasgow's modified eagle medium (GMEM), SMIP-8, modified eagle medium. (MEM), VP-SFM, DMEM based SFM, DMEM/F12, DMEM/Ham's F12, VPSFM/William's medium E, ExCell 525(SFM), adenovirus expression medium (AEM) and Excell 65629 (Genzel et al., 2009). It will be appreciated by persons skilled in the art that such mediums may be supplemented with additional growth factors, for example, but not limited, amino acids, hormones, vitamins and minerals. Optionally, such mediums may be supplemented with serum, for example fetal calf serum.

In one example, the cells are cultured using the hatch cell culture process. In one example, the cells are cultured using the perfusion cell culture process. In one example, the cells are cultured in a seed medium and a production medium. In one example, the cells are cultured in a stirred-tank reactor. In one example, the volume of the reactor is from about 1 L to about 2500 L. In one example, the cells are cultured in a wave bioreactor. In one example, the cells are cultured in a cell factory system e.g. a Nunc cell factory system (Genzel et al., 2009).

Antigens which Stimulate an Immune Response

The present invention provides for use of compounds with modify C6orf106 protein activity in combination with at least one antigen which stimulates an immune response. The compounds can be mixed with or conjugated to the at least one antigen to generate a protective immune response when the compound is administered to a subject. In an embodiment, a compound which decreases C6orf106 activity is administered with at least one antigen or vaccine composition to increase the immune response to the at least one antigen or vaccine composition.

By “at least one antigen” it is meant one or more antigen types or antigenic determinants. In an embodiment, the antigen may by conjugated to a compound of the invention.

A composition comprising the compound conjugated to the antigen or a combination of the compound and the antigen can be administered to a subject that has or is susceptible to, or at risk for a condition.

As used herein, an “antigen” means a substance that has the ability to induce a specific immune response. The antigen may be a whole organism in any of its life cycle stages, inactivated whole organism, fragments or components isolated from the whole organism, lysate of the organism or tumor lysate, specific antigens genetically or synthetically engineered through methods known in the art. In addition, the selected antigen may be derived from either or both a mature whole organism or sporozoites (oocysts).

The antigen for use in methods of the present invention can also consist of whole cells or sub-cellular fractions thereof. Such cells or sub-cellular fractions thereof may be derived from, for example, a tumor or infected tissue.

Preferred selected antigens include, for example, antigens from:
pollens;
allergens, especially those that induce asthma;
viruses, such as those described herein, and in particular influenza, feline leukemia virus, feline immunodeficiency virus, HIV-1, HIV-2, rabies, measles, hepatitis B, hoof and mouth disease, papilloma virus, cytomegalovirus, herpes simplex, hepatitis A, hepatitis C, HTLV-1 and HTLV-2;
bacteria, such as the ethiological agents of anthrax, leprosy, tuberculosis, diphtheria, Lyme disease, syphilis, typhoid fever, and gonorrhea;
protozoans, such as Babeosis bovis, Plasmodium, Leishmania spp. Toxoplasma gondii, and Trypanosoma cruzi;
fungi, such as Aspergillus sp., Candida albicans, Cryptococcus neoformans, and Histoplasma capsulatum;
parasites such as helminths; and
tumor antigens, such as mucin-1 (MUC-1), carcinoembryonic antigen, prostate-specific membrane antigen, prostate specific antigen, protein MZ2-E, polymorphic epithelial mucin (PEM), folate-binding-protein LK26, truncated epidermal growth factor receptor (EGRF), Thomsen-Friedenreich (T) antigen, telomerase, survivin, Melan-A/MART-1, WT1, LMP2, human papillomavirus (HPV) E6 E7, human epithelial growth factor receptor (HER-2/neu), Idiotype, melanoma associated antigen 3 (MAGE-3), p53, NY-ESO-1, prostatic acid phosphatase (PAP), cancer testis antigens, 5T4, and GM-2 and GD-2 gangliosides.

The antigen can be a protein, peptide, polysaccharide or oligosaccharide (free or conjugated to a protein carrier), or mixtures thereof. The proteins and peptides may be part of an extract or lysate, purified from a natural source, synthesized by means of solid phase synthesis, or may be obtained by means of recombinant genetics. The polysaccharides and oligosaccharides may be isolated from a natural source, or may be synthesized using enzymatic procedures and/or organic synthesis approaches.

An antigen may form part of a fusion protein in order to facilitate expression and purification on production of the fusion protein in recombinant host cells. The non-antigen portion of the fusion protein would generally represent the N-terminal region of the fusion polypeptide with the carboxy terminal sequences comprising antigen sequences. Fusion proteins may be selected from glutathione-S-transferase, β-galactosidase, or any other protein or part thereof, particularly those which enable affinity purification utilizing the binding or other affinity characteristics of the protein to purify the resultant fusion protein. The protein may also be fused to the C-terminal or N-terminal of the carrier protein. The nature of the fusion protein will depend upon the vector system in which fusion proteins are produced. An example of a bacterial expression vector is pGEX, which on subcloning of a gene of interest into this vector produces a fusion protein consisting of glutathione-transferase with the protein of interest.

Alternatively, synthetic peptides or polypeptides, optionally coupled to a protein carrier may be used in the invention. Synthetic peptides or polypeptides may be produced in accordance with standard methods.

Useful peptides or polypeptides may comprise an epitope-bearing portion of a polypeptide known to elicit an antibody and/or an antigen-specific CTL response when the whole polypeptide is administered to an animal. The epitope of this polypeptide portion is an immunogenic or antigenic epitope of the polypeptide.

An “immunogenic epitope” is defined as a part of a protein that elicits an antibody and/or an antigen-specific CTL response when the whole protein is the immunogen. On the other hand, a region of a protein molecule to which an antibody or MHC molecule can bind is defined as an “antigenic epitope”. The number of immunogenic epitopes of a protein generally is less than the number of antigenic epitopes.

With regard to the selection of peptides or polypeptides bearing an antigenic epitope, it is well known in that art that relatively short synthetic peptides that mimic part of a protein sequence routinely elicit antiserum that reacts with the partially mimicked protein (see, for example, Sutcliffe et al., 1983). Peptides capable of eliciting protein-reactive sera are frequently represented in the primary sequence of a protein, can be characterized by a set of simple chemical rules, and are confined neither to immunodominant regions of intact proteins (i.e., immunogenic epitopes) nor to the amino or carboxyl terminals.

Antigenic epitope-bearing peptides and polypeptides of the invention preferably contain a sequence of at least seven, more preferably at least nine and most preferably between about 15 to 30 amino acids contained within the amino acid sequence of a particular polypeptide.

Epitopes recognized by the T-cell receptors on CTLs may be different from those seen by antibodies. Usually, CTLs recognize peptides (derived from proteins enzymatically degraded in the cytosol compartment) which are bound to MHC class I molecules and exposed on the cell surface. These CTL-recognized peptides bind selectively to MHC class I molecules according to MHC allele-specific sequence motifs. These peptides can be identified by expression cloning (see, van der Bruggen, et al., 1991) and predicted using various class I and class II binding peptide algorithms (Pietersz et al., 2006).

Alternatively,CTL-recognized peptides can be identified by induction of cytotoxic T lymphocytes by in vitro or ex vivo stimulation with peptides derived from the protein antigen used for immunization. The particular CTL-recognized epitope-bearing peptides and polypeptides of the invention are preferably sequences of at least six amino acids, and more preferably between about 7 to 20 amino acids.

Epitope-bearing peptides and polypeptides may be produced by any conventional means.

### Bacterial Antigens

The antigen can be derived from bacteria, including but not limited to, Helicobacter pylori, Chlamydia pneumoniae, Chlamydia trachomatis, Ureaplasma urealyticum, Mycoplasma pneumoniae, Staphylococcus spp., Staphylococcus aureus, Streptococcus spp., Streptococcus pyogenes, Streptococcus pneumoniae, Streptococcus viridans, Enterococcus faecalis, Neisseria meningitidis, Neisseria gonorrhoeae, Bacillus anthracis, Salmonella spp., Salmonella typhi, Vibrio cholera, Pasteurella pestis, Pseudomonas aeruginosa, Campylobacter spp., Campylobacter jejuni, Clostridium spp., Clostridium difficile, Mycobacterium spp., Mycobacterium tuberculosis, Treponema spp., Borrelia spp., Borrelia burgdorferi, Leptospira spp., Hemophilus ducreyi, Corynebacterium diphtheria, Bordetella pertussis, Bordetella parapertussis, Bordetella bronchiseptica, hemophilus influenza, Escherichia coli, Shigella spp., Erlichia spp., and Rickettsia spp.

The bacterial antigen can be native, recombinant or synthetic. Such bacterial antigens include, but are not limited to, selectins or lectins from bacteria that bind to carbohydrate determinants present on cell surfaces, and bacteria receptors for proteins, such as fibronectin, laminin, and collagens.

### Viral Antigens

The antigen can be derived from viruses, including but not limited to, Influenza viruses, a Parainfluenza viruses, Mumps virus, Adenoviruses, Respiratory syncytial virus, Epstein-Barr virus, Rhinoviruses, Polioviruses, Coxsackieviruses, Echoviruses, Rubeola virus, Rubella virus, Varicell-zoster virus, Herpes viruses (human and animal), Herpes simplex virus, Parvoviruses (human and animal), Cytomegalovirus, Hepatitis viruses, Human papillomavirus, Alphaviruses, Bunyaviruses, Rabies virus, Arenaviruses, Filoviruses, Bornaviriade, HIV 1, HIV 2, HTLV-1, FeLV, Bovine LV, FeIV, Canine distemper virus, Canine contagious hepatitis virus, Feline calicivirus, Feline rhinotracheitis virus, TGE virus (swine), and Foot and mouth disease and other viruses as herein described.

Viral antigens can be native, recombinant or synthetic. Such viral antigens include, but are not limited to, viral proteins that are responsible for attachment to cell surface receptors to initiate the infection process, such as (i) envelope glycoproteins of retroviruses (HIV, HTLV, FeLV and others) and herpes viruses, and (ii) the neuramidase of influenza viruses.

### Tumor Antigens

In an embodiment, of the invention, the subject has cancer or is at increased risk of developing cancer.

A method of treating and/or preventing cancer of the present invention may comprise one or more tumor associated antigens. Tumor associated antigens can be native, recombinant or synthetic. Such tumor associated antigens include, but are not limited to, MUC-1 and peptide fragments thereof, protein MZ2-E, polymorphic epithelial mucin, folate-binding protein LK26, MAGE-1 or MAGE-3 and peptide fragments thereof, Human chorionic gonadotropin (HCG) and peptide fragments thereof, Carcinoembryonic antigen (CEA) and peptide fragments thereof, Alpha fetoprotein (AFP) and peptide fragments thereof, Pancreatic oncofetal antigen and peptide fragments thereof, CA 125, 15-3, 19-9, 549, 195 and peptide fragments thereof, Prostate-specific antigens (PSA) and peptide fragments thereof, Prostate-specific membrane antigen (PSMA) and peptide fragments thereof, Squamous cell carcinoma antigen (SCCA) and peptide fragments thereof, Ovarian cancer antigen (OCA) and peptide fragments thereof, Pancreas cancer associated antigen (PaA) and peptide fragments thereof, Her1/neu and peptide fragments thereof, gp-100 and peptide fragments thereof, mutant K-ras proteins and peptide fragments thereof, mutant p53 and peptide fragments thereof, nonmutant p53 and peptide fragments thereof, truncated epidermal growth factor receptor (EGFR), chimeric protein p210BCR-ABL, telomerase and peptide fragments thereof, suvivin and peptide fragments thereof, Melan-A/MART-1 protein and peptide fragments thereof, WT1 protein and peptide fragments, LMP2 protein and peptide fragments, HPV E6 E7 protein and peptide fragments, HER-2/neu protein and peptide fragments, Idiotype protein and peptide fragments, NY-ESO-1 protein and peptide fragments, PAP protein and peptide fragments, cancer testis proteins and peptide fragments, and 5T4 protein and peptide fragments. Other exemplary tumor antigens (Cheever et al., 2009).

### Conditions

Exemplary conditions to be treated and/or prevented by modulating an immune response with a method or compound of the present invention include: an infection, an immunodeficiency, an autoimmune disease, an inflammatory condition and cancer. Exemplary conditions may have an inappropriately increased immune response or an inappropriately decreased immune response which requires modulation. Exemplary conditions may have an inappropriately increased or decreased IFN response which requires modulation.

### Infection

In one embodiment, a condition to be treated and/or prevented is an infection. In one embodiment, the infection is a viral, bacterial, fungal or protozoan infection. In an embodiment, the infection is a viral infection. The virus can be any virus wherein the immune response to the virus is increased by reduced C6orf106 protein activity. In an embodiment, the virus is an animal virus. In an embodiment, the animal virus is a human virus. In an embodiment, the virus is a non-human virus. In an embodiment, the virus is a negative-strand RNA virus.

Examples of viruses for use in the present include members of the Mononegavirales, Herpesvirales and Nidovirales orders. In an embodiment, the virus is from the order Mononegavirales. In an embodiment, the Mononegavirales is selected from: Paramyxoviridae, Rhabdoviridae, Filoviridae and Bornaviriade.

Examples of viruses for use in the present invention include, but are not limited to, viruses in a family selected from a: Orthomyxoviridae, Retroviridae, Herpesviridae, Paramyxoviridae, Rhabdoviridae, Bornaviriade and Coronaviridae.

The Orthomyxoviridae virus may be, for example, an influenza A virus, an Influenza B virus, and Influenza C virus, Isavirus, Thogotovirus and/or Quaranjavirus. The influenza virus may be an Influenza A virus. The Influenza A virus may be selected from Influenza A viruses isolated from an animal. In an embodiment, the animal is a human or an avian. In particular, the Influenza A virus may be selected from H1N1, H1N3, H1N4, H1N5, H1N6, H1N7, H1N9, H2N1, H2N2, H2N3, H2N4, H2N5, H2N7, H2N8, H2N9, H3N1, H3N2, H3N3, H3N4, H3N5, H3N6, H3N8, H4N1, H4N2, H4N3, H4N4, H4N5, H4N6, H4N8, H4N9, H5N1, H5N2, H5N3, H5N6, H5N7, H5N8, H5N9, H6N1, H6N2, H6N3, H6N4, H6N5, H6N6, H6N7, H6N8, H6N9, H7N1, H7N2, H7N3, H7N4, H7N5, H7N7, H7N8, H7N9, H9N1, H9N2, H9N3, H9N5, H9N6, H9N7, H9N8, H10N1, H10N3, H10N4, H10N6, H10N7, H10N8, H10N9, H11N2, H11N3, H11N6, H11N9, H12N1, H12N4, H12N5, H12N9, H13N2, H13N6, H13N8, H13N9, H14N5, H15N2, H15N8, H15N9 and H16N3. In one embodiment, the Influenza A virus is selected from H1N1, H3N2, H7N7, and/or H5N1.

The Retroviridae may be, for example, the Human immunodeficiency virus.

The Herpesviridae virus may be, for example, a HSV-1, HSV-2, Varicella zoster virus, Epstein-barr virus or Cytomegalovirus.

The Paramyxoviridae virus may be, for example, a Paramyxovirmae or Pneumovirinae, In an embodiment, the Paramyxovirinae may be, for example an/a Aquaparamyxovirus, Avulavirus, Ferlavirus, Henipavirus, Morbillivirus, Newcastle disease virus, Respirovirus, or Rubulavirus. In an embodiment, the Pneumovirinae may be, for example, a Metapneumovirus or Orthopneumovirus. In an embodiment, the Paramyxoviridae is Newcastle disease virus.

The Rhabdoviridae may be, for example, a Lyssavirus, Novirhabdovirus Ephemerovirus, Perhabdovirus, Tibrovirusk, Nucleohabdovirus, Tupavirus Vesiculovirus, Sprivivirus, Cytorhabdovirus, or a Sigmavirus. In an embodiment, the Rhabdoviridae may be the Rabies virus, Bas-Congo Virus, Carajas virus, Chandipura virus, Cocal virus, Isfahan virus, Maraba virus, Piry virus, Vesicular stomatitis virus, Bovine ephemeral fever virus, Tibrogargan virus, Durham virus, Perch rhabdovirus, Spring viraemia of carp virus, Hirame rhabdovirus, Infectious hematopoietic necrosis virus, Viral hemorrhagic septicemia virus or the Snakehead rhabdovirus. In an embodiment, the Vesicular stomatitis virus is the Vesicular stomatitis Alagoas virus, Vesicular stomatitis Indiana virus, or the Vesicular stomatitis New Jersey virus.

The Filoviridae may be, for example, Ebolavirus, Cuevavirus or Marburg virus. The Ebola virus may be, for example Bundibugyo ebolavirus, Reston ebolavirus, Sudan ebolavirus, Taï Forest ebolavirus or Zaire ebolavirus.

The Bornaviriade may be for example, a Elapid 1 bornavirus, Mammalian 1 bornavirus, Passeriform 1 bornavirus, Passeriform 2 bornavirus, Psittaciform 1 bornavirus, Psittaciform 2 bornavirus or Waterbird 1 bornavirus.

The Coronaviradae virus may be, for example, a Coronavirinae or a Corovirinae. The Coronavirinae may be a Alphacoronavirus, Betacoronavirus, Deltacoronavirus, or Gammacoronavirus. The Torovirinae may be a Alphacoronavirus or Betacoronavirus. In one embodiment, the Coronaviradae may be the SARS (severe acute respiratory syndrome) coronavirus.

In an embodiment, the virus in selected from: Influenza virus, Canine distemper virus, Measles virus, Reovirus, Eastern equine encephalitis virus, Canine parainfluenza virus, Rabies virus, Fowlpox virus, Western equine encephalitis virus, Mumps virus, Equine encephalomyelitis, Rubella virus, Egg drop syndrome virus, Avian oncolytic viruses, Avian infectious laryngotracheitis Herpesvirus, Newcastle disease virus, Bovine parainfluenza virus, Smallpox virus, Infectious bursal disease, Bovine Ibaraki virus, Recombinant poxvirus, Avian adenovirus type I, II or III, Swine Japanese encephalitis virus, Yellow fever virus, Herpes virus, Sindbis virus, Infections bronchitis virus, Semliki forest virus, Encephalomyelitis virus, Venezuelan EEV virus, Chicken anaemia virus, Marek's disease virus, Parvovirus, Foot and mouth disease virus, Porcine reproductive and respiratory syndrome virus, Classical swine fever virus, Bluetongue virus, Kabane virus, Infectious salmon anaemia virus, Infectious hematopoietic necrosis virus, Viral haemorrhagic septicaemia virus, Bundibugyo ebolavirus, Reston ebolavirus, Sudan ebolavirus, Taï Forest ebolavirus, Zaire ebolavirus, Marburg, SARS and Infectious pancreatic necrosis virus.

In an embodiment, the infection is a bacterial infection. In an embodiment, the bacteria is selected from: Helicobacter pylori, Chlamydia pneumoniae, Chlamydia trachomatis, Ureaplasma urealyticum, Mycoplasma pneumoniae, Staphylococcus spp., Staphylococcus aureus, Streptococcus spp., Streptococcus pyogenes, Streptococcus pneumoniae, Streptococcus viridans, Enterococcus faecalis, Neisseria meningitidis, Neisseria gonorrhoeae, Bacillus anthracis, Salmonella spp., Salmonella typhi, Vibrio cholera, Pasteurella pestis, Pseudomonas aeruginosa, Campylobacter spp., Campylobacter jejuni, Clostridium spp., Clostridium difficile, Mycobacterium spp., Mycobacterium tuberculosis, Treponema spp., Borrelia spp., Borrelia burgdorferi, Leptospira spp., Hemophilus ducreyi, Corynebacterium diphtheria, Bordetella pertussis, Bordetella parapertussis, Bordetella bronchiseptica, hemophilus influenza, Escherichia coli, Shigella spp., Erlichia spp., and Rickettsia spp.

In an embodiment, the infection is a fungal infection. In an embodiment, the fungal infection is selected from: Aspergillus sp., Candida albicans, Cryptococcus neoformans, and Histoplasma capsulatum.

In an embodiment, the infection is a protozoan infection. In an embodiment, the protozoan is selected from: Babeosis bovis, Plasmodium, Leishmania spp. Toxoplasma gondii, and Trypanosoma cruzi.

### Immunodeficiency

In one example, a condition to be treated and/or prevented is an immunodeficiency. In one example, the immunodeficiency is selected from: X-linked agammaglobulinemia, common variable immunodeficiency, severe combined immunodeficiency, acquired immune deficiency syndrome, leukemia, human immunodeficiency virus, viral hepatitis, such as hepatitis C and multiple myeloma.

### Autoimmune Disease

In one example, a condition to be treated and/or prevented is an autoimmune disease. In one example, a patient with an autoimmune disease is treated with a compound which increases C6orf106 protein activity and where the immune response and/or cytokine production is reduced in a subject. In an embodiment, the autoimmune disease is characterized by chronic inflammation. In an embodiment, the autoimmune disease is characterized by increased levels of inflammation. In an embodiment, the autoimmune disease is characterized by an increased IFN response. In an embodiment, the autoimmune disease is characterized by an increased type I IFN response.

For example, the autoimmune disease is selected from: Acute Disseminated Encephalomyelitis (ADEM), Acute necrotizing hemorrhagic leukoencephalitis, Addison's disease, Agammaglobulinemia, Amyloidosis, Ankylosing spondylitis, Anti-GBM/Anti-TBM nephritis, Antiphospholipid syndrome (APS), Autoimmune angioedema, Autoimmune aplastic anemia, Autoimmune dysautonomia, Autoimmune hepatitis, Autoimmune hyperlipidemia, Autoimmune, immunodeficiency, Autoimmune inner ear disease (AIED), Autoimmune myocarditis, Autoimmune oophoritis, Autoimmune pancreatitis, Autoimmune retinopathy, Autoimmune thrombocytopenic purpura (ATP), Autoimmune thyroid disease, Autoimmune urticarial, Axonal & neuronal neuropathies, Balo disease, Behcet's disease, Bullous pemphigoid, Cardiomyopathy, Castleman disease, Celiac disease, Chagas disease, Chronic inflammatory demyelinating polyneuropathy (CIDP), Chronic recurrent multifocal ostomyetitis (CRMO), Churg-Strauss syndrome, Cicatricial pemphigoid/benign mucosal pemphigoid, Crohn's disease, Cogans syndrome, Coxsackie myocarditis, CREST disease, Essential mixed cryoglobulinemia, Demyelinating neuropathies, Dermatitis herpetiformis, Dermatomyositis, Devic's disease (neuromyelitis optica), Discoid lupus, Dressler's syndrome, Fibrosing alveolitis, Giant cell arteritis (temporal arteritis), Giant cell myocarditis, Glomerulonephritis, Goodpasture's syndrome, Granulomatosis with Polyangiitis (GPA) (formerly called Wegener's Granulomatosis), Graves' disease, Guillain-Barre syndrome, Hashimoto's encephalitis, Hashimoto's thyroiditis, Hemolytic anemia, Henoch-Schonlein purpure, Hypogammaglobulinemia, Idiopathic thrombocytopenic purpura (ITP), IgA nephropathy, IgG4-related sclerosing disease, Inclusion body myositis, Interstitial cystitis, Irritable bowel syndrome, Juvenile arthritis, Juvenile diabetes (Type I diabetes), Juvenile myositis, Kawasaki syndrome, Lambert-Eaton syndrome, Leukocytoclastic vasculitis, Lichen planus, Lichen sclerosus, Ligneous conjunctivitis, Linear IgA disease (LAD), Lupus (SLE), Lyme disease, chronic, Meniere's disease, Microscopic polyangiitis, Mixed connective tissue disease (MCTD), Mooren's ulcer, Mucha-Habermann disease, Multiple sclerosis, Myasthenia gravis, Myositis, Narcolepsy, Neuromyelitis optica (Devic's), Neutropenia, Ocular cicatricial pemphigoid, Optic neuritis, Palindromic rheumatism, PANDAS (Pediatric Autoimmune Neuropsychiatric Disorders Associated with Streptococcus), Paraneoplastic cerebellar degeneration, Paroxysmal nocturnal hemoglobinuria (PNH), Parry Romberg syndrome, Parsonnage-Turner syndrome, Pars planitis (peripheral uveitis), Pemphigus, Peripheral neuropathy, Perivenous encephalomyelitis, Pernicious anemia, POEMS syndrome, Polyarteritis nodosa, Type I, II, & III autoimmune polyglandular syndromes, Polymyalgia rheumatic, Polymyositis, Postmyocardial infarction syndrome, Postpericardiotomy syndrome, Progesterone dermatitis, Primary biliary cirrhosis, Primary sclerosing cholangitis, Psoriasis, Psoriatic arthritis, Idiopathic pulmonary fibrosis, Pyoderma gangrenosum, Pure red cell aplasia, Raynauds phenomenon, Reactive Arthritis, Reflex sympathetic dystrophy, Reiter's syndrome, Relapsing polychondritis, Retroperitoneal fibrosis, Rheumatic fever, Rheumatoid arthritis, Sarcoidosis, Schmidt syndrome, Scleritis, Scleroderma, Sjogren's syndrome, Sperm & testicular autoimmunity, Stiff person syndrome, Subacute bacterial endocarditis (SBE), Susac's syndrome, Sympathetic ophthalmia, Takayasu's arteritis, Temporal arteritis/Giant cell arteritis, Thrombocytopenic purpura (TTP), Tolosa-Hunt syndrome, Transverse myelitis, Type 1 diabetes, Ulcerative colitis, Undifferentiated connective tissue disease (UCTD), Uveitis, Vasculitis, Vesiculobullous dermatosis, Vitiligo, and Wegener's granulomatosis.

In one example, the autoimmune disease is selected from Ulcerative colitis, Crohn's disease, Irritable bowel syndrome, Rheumatoid arthritis, Polyarthritis, Multiple sclerosis, Uveitis, asthma, Type 1 diabetes, Type 2 diabetes, Lupus or Chronic obstructive pulmonary disease.

### Inflammatory Conditions

In one example, the condition to be treated and/or prevented is an inflammatory condition. In one example, the inflammatory condition is any condition where inflammation is reduced by administration of a compound which increases C6orf106 protein activity and wherein the immune response and/or cytokine response is reduced. In an embodiment, the condition is chronic. In one embodiment, the inflammatory condition is caused by one of the aforementioned autoimmune disease. In one embodiment, the inflammation is caused by a trauma, for example trauma caused by injury, burns or frostbite.

### Cancer

In one example, the condition to be treated and/or prevented is cancer. As used herein “cancer” it is meant any of various malignant neoplasms, characterized by the proliferation of cells that have the capability to invade surrounding tissue and/or metastasize to new colonisation sites. In one embodiment, the cancer may be, for example, bladder cancer, bone cancer, bowel cancer, brain cancer, breast cancer, colorectal cancer, cancer of unknown primary, cervical cancer, gastric cancer, head and neck cancer, kidney cancer, leukemia, liver cancer, lung cancer, lymphoma, mesothelioma, myeloma, ovarian cancer, pancreatic cancer, prostate cancer, skin cancer, stomach cancer, testicular cancer, thyroid cancer, uterine cancer, vaginal cancer, vulvar cancer. In an embodiment, the cancer is a carcinoma, sarcoma, lymphoma, germ cell tumor, or a blastoma.

### Methods of Identifying Compounds

In an embodiment, compounds of the present invention which modify C6orf106 protein activity can be identified by screening. Such compounds can be identified by any method known to a person skilled in the art and may include one or more or all of i) assessing the ability of a compound to bind the C6orf106 gene or C6orf106 mRNA, ii) assessing the ability of the compound to modify C6orf106 gene expression, iii) assessing the ability of a compound to bind the C6orf106 protein, iv) assessing the ability of the compound to modify C6orf106 activity in a cell. Assessing the ability of the compound to modify C6orf106 activity may involve assessing the ability of the compound to modulate the immune, response to an infection, such as a viral invention. Alternatively, assessing the ability of a compound to modify C6orf106 activity may involve assessing the ability of a compound to induce an immune response. Assessing an immune response, may involve, but is not limited to, measuring the level of cytokines in response to treatment by a compound. In an embodiment, measuring an immune response involves measuring the level of the gene and/or protein of one or more or all of IFN-α, IFN-β and TNF-α.

### C6orf106 Gene Expression

In an embodiment, a compound may decrease C6orf106 protein activity by reducing the expression of the C6orf106 gene. In an embodiment, a compound may increase C6orf106 protein activity by increasing the expression of the C6orf106 gene or a fragment thereof. Such compounds may be screened in vitro, in ovo or in vivo. An in vitro experiment, may include administering a candidate compound for use in a method of the invention to a cell and assessing the level of C6orf106 mRNA expression before and after administration of the compound. In vitro studies may be performed using any suitable cell line. In an embodiment, the cell line is a mammalian cell line. In an embodiment the cell line is a human cell line, such as a HeLa cell line. In ovo studies may be performed in an avian egg and in vivo studies may be performed using a mammalian models, such as a mouse model. Gene expression may be measured using real time PCR.

Any suitable technique that allows for the detection of a nucleic acid may be used, including those that allow quantitative assessment of the level of expression of the C6orf106 gene in a tissue and/or cell. For example, levels of a transcribed gene can be determined by Northern blotting, and/or RT-PCR. With the advent of quantitative (real-time) PCR, the number of transcript copies present in any RNA population can accurately be determined by using appropriate primers for the gene of interest. The nucleic acid may be labelled and hybridised on a gene array, in which case the gene concentration will be directly proportional to the intensity of the radioactive or fluorescent signal generated in the array.

The “polymerase chain reaction” (“PCR”) is a reaction in which replicate copies are made of a target polynucleotide using a “pair of primers” or “set of primers” consisting of an “upstream” and a “downstream” primer, and a catalyst of polymerization, such as a DNA polymerase, and typically a thermally-stable polymerase enzyme. Methods for PCR are known in the art, and are taught, for example, in “PCR” (Ed. M. J. McPherson and S. G. Moller (2000) BIOS Scientific Publishers Ltd, Oxford). PCR can be performed on cDNA obtained from reverse transcribing mRNA isolated from biological samples.

Another nucleic acid amplification technique is reverse transcription polymerase chain reaction (qRT-PCR). First, complementary DNA (cDNA) is made from an RNA template, using a reverse transcriptase enzyme, and then PCR is performed on the resultant cDNA.

Another method for amplification is the ligase chain reaction (“LCR”), disclosed in EP0320308.

Qβ Replicase, may also be used as still another amplification method in the present invention. In this method, a replicative sequence of RNA that has a region complementary to that of a target is added to a sample in the presence of an RNA polymerase. The polymerase will copy the replicative sequence that can then be detected.

An isothermal amplification method, in which restriction endonucleases and ligases are used to achieve the amplification of target molecules that contain nucleotide 5′α-thio-triphosphates in one strand of a restriction site may also be useful in the amplification of nucleic acids in the present invention.

Strand Displacement Amplification (SDA) is another method of carrying out isothermal amplification of nucleic acids which involves multiple rounds of strand displacement and synthesis, i.e., nick translation.

Target specific sequences can also be detected using a cyclic probe reaction (CPR). In CPR, a probe having 3′ and 5′ sequences of non-specific DNA and a middle sequence of specific RNA is hybridised to DNA that is present in a sample. Upon hybridisation, the reaction is treated with RNase H, and the products of the probe identified as distinctive products that are released after digestion. The original template is annealed to another cycling probe and the reaction is repeated.

Another example of an isothermal amplification technique is LAMP (loop-mediated isothermal amplification of DNA).

Further amplification methods are described in GB Application No. 2 202 328, and in PCT Application No. PCT/US89/01025, and may be used in accordance with the present invention.

Other nucleic acid amplification procedures include transcription-based amplification systems (TAS), including nucleic acid sequence based amplification (NASBA) and 3SR (WO 88/10315).

Methods for direct sequencing of nucleotide sequences are well known to those skilled in the art and can be found for example in Ausubel et al. eds., Short Protocols in Molecular Biology, 3rd ed., Wiley, (1995) and Sambrook et al. Molecular Cloning, 2nd ed., Chap. 13, Cold Spring Harbor Laboratory Press, (1989). Sequencing can be carried out by any suitable method, for example, dideoxy sequencing, chemical sequencing, next generation sequencing techniques or variations thereof. Direct sequencing has the advantage of determining variation in any base pair of a particular sequence.

Hybridization based detection systems include, but are not limited to, the TaqMan assay and molecular beacons. The TaqMan assay (U.S. Pat. No. 5,962,233) uses allele specific (ASO) probes with a donor dye on one end and an acceptor dye on the other end such that the dye pair interact via fluorescence resonance energy transfer (FRET).

### C6orf106 Protein Activity

In an embodiment, a compound of the present invention may modulate C6orf106 protein activity by reducing or increasing expression of the protein or modifying the activity of the protein. Such compounds may be screened for the ability to bind to and modulate the level of the activity of the protein by any method known to a person skilled in the art. Such methods may include, an immunoassay.

The compound is capable of binding C6orf106 in the presence of excess quantities of other polypeptides. The parameters required to achieve such specificity can be determined routinely, using conventional methods in the art. Preferably, the binding agent binds to C6orf106, at least one times, or at least two times, or at least three times, or at least four times, or at least five times the background and more typically 10 to 100 times background.

Screening assays contemplated herein include any known assay for detecting proteins in a biological sample isolated from a subject, such as, for example, SDS/PAGE, isoelectric focussing, 2-dimensional gel electrophoresis comprising SDS/PAGE and isoelectric focussing, an immunoassay, a detection based system using an antibody or non-antibody ligand of the protein, such as, for example, a small molecule (e.g. a chemical compound, agonist, antagonist, allosteric modulator, competitive inhibitor, or non-competitive inhibitor, of the protein). In accordance with these embodiments, the antibody or small molecule may be used in any standard solid phase or solution phase assay format amenable to the detection of proteins. Optical or fluorescent detection, such as, for example, fluorescence-activated cell sorting (FACS), using mass spectrometry, MALDI-TOF, biosensor technology, evanescent fiber optics, or fluorescence resonance energy transfer, is clearly encompassed by the present invention. Assay systems suitable for use in high throughput screening of mass samples, particularly a high throughput spectroscopy resonance method (e.g. MALDI-TOF, electrospray MS or nano-electrospray MS), are also contemplated.

Suitable immunoassay formats include immunoblot, Western blot, dot blot, enzyme linked immunosorbent assay (ELISA), radioimmunoassay (RIA) and enzyme immunoassay. Modified immunoassays utilizing fluorescence resonance energy transfer (FRET), isotope-coded affinity tags (ICAT), matrix-assisted laser desorption/ionization time of flight (MALDI-TOF), electrospray ionization (ESI), biosensor technology, evanescent fiber-optics technology or protein chip technology are also useful.

In one embodiment, the assay is a semi-quantitative assay or quantitative assay.

Standard solid phase ELISA formats are particularly useful in determining the concentration of a protein or antibody from a variety of samples.

Such ELISA based systems are particularly suitable for quantification of the amount of a protein or antibody in a sample, such as, for example, by calibrating the detection system against known amounts of a standard.

In another form, an ELISA consists of immobilizing an antibody that specifically binds C6orf106 on a solid matrix, such as, for example, a membrane, a polystyrene or polycarbonate microwell, a polystyrene or polycarbonate dipstick or a glass support. A sample is then brought into physical relation with said antibody, and the antigen in the sample is bound or ‘captured’. The bound protein can then be detected using a labelled antibody. Alternatively, a third labelled antibody can be used that binds the second (detecting) antibody.

In Silico Screening

In an embodiment, such compounds can be identified by in silico screening by any method known to a skilled individual. Known techniques of this sort include, but are not limited to those provided in Sheridan and Venkataraghavan, (1987), Goodford, (1984), Beddell, (1985), Hol, (1986), Verlinde and Hol, (1984), Walters et al., (1998), Langer and Hoffmann, (2001), Good, (2001), Gane and Dean, (2000), Zhang et al., (2015), Cerqueira et al., (2015), Kuenemann et al., (2015), Westermaier et at., (2015).

For a compound to bind a C6orf106 protein, it will typically require a suitable level of stereochemical complementarity. In general, the design of a molecule possessing stereochemical complementarity can be accomplished by means of techniques that optimize, chemically and/or geometrically, the “fit” between a molecule and a target receptor. There are at least two approaches to designing a molecule, according to the present invention, that complements the stereochemistry of a C6orf106 protein.

The first approach is to in silico directly dock molecules from a three dimensional structural database, to the receptor site, using mostly, but not exclusively, geometric criteria to assess the goodness of fit of a particular molecule to the site. In this approach, the number of internal degrees of freedom (and the corresponding local minima in the molecular conformation space) is reduced by considering only the geometric (hard sphere) interactions of two rigid bodies, where one body (the active site) contains “pockets” or “grooves” that form binding sites for the second body (the complementing molecule, as ligand).

This approach is illustrated by Kuntz et al. (1982) and Ewing et al. (2001), whose algorithm for ligand design is implemented in a commercial software package, DOCK version 4.0, distributed by the Regents of the University of California and further described in a document, provided by the distributor, which is entitled “Overview of the DOCK program suite” the contents of which are hereby incorporated by reference. Pursuant to the Kuntz algorithm, the shape of a region of interest is defined as a series of overlapping spheres of different radii. One or more extant databases of crystallographic data, such as the Cambridge Structural Database System maintained by Cambridge University (University Chemical Laboratory, Lensfield Road, Cambridge CB2 1EW, U.K.), the Protein Data Bank maintained by the Research Collaboratory for Structural Bioinformatics (Rutgers University, N.J., U.S.A.), LeadQuest (Tripos Associates, Inc., St. Louis, Mo.), Available Chemicals Directory (Molecular Design Ltd., San Leandro, Calif.), and the NCI database (National Cancer Institute, U.S.A) is then searched for molecules which approximate the shape thus defined.

Molecules identified in this way, on the basis of geometric parameters, can then be modified to satisfy criteria associated with chemical complementarity, such as hydrogen bonding, ionic interactions and Van der Waals interactions. Different scoring functions can be employed to rank and select the best molecule from a database (see, for example, Bohm and Stahl, 1999). The software package FlexX, marketed by Tripos Associates, Inc. (St. Louis, Mo.) is another program that can be used in this direct docking approach (Rarey et al., 1996).

The second preferred approach entails an assessment of the interaction of respective chemical groups (“probes”) with the active site at sample positions within and around the site, resulting in an array of energy values from which three dimensional contour surfaces at selected energy levels can be generated. The chemical probe approach to ligand design is described, for example, by Goodford (1985), and is implemented in several commercial software packages, such as GRID (product of Molecular Discovery Ltd., West Way House, Elms Parade, Oxford. OX2 9LL, U.K.). Pursuant to this approach, the chemical prerequisites for a site complementing molecule are identified at the outset, by probing the active site with different chemical probes, e.g., water, a methyl group, an amine nitrogen, a carboxyl oxygen, and a hydroxyl. Favoured sites for interaction between the active site and each probe are thus determined, and from the resulting three dimensional pattern of such sites a putative complementary molecule can be generated. This may be done either by programs that can search three dimensional databases to identify molecules incorporating desired pharmacophore patterns or by programs which using the favoured sites and probes as input perform de novo design.

Programs suitable for searching three dimensional databases to identify molecules bearing a desired pharmacophore include: MACCS 3D and ISIS/3D (Molecular Design Ltd., San Leandro, Calif.), ChemDBS 3D (Chemical Design Ltd., Oxford, U.K.), and Sybyl/3DB Unity (Tripos Associates, Inc., St. Louis, Mo.).

Databases of chemical structures are available from a number of sources including Cambridge Crystallographic Data Centre (Cambridge, U.K.), Molecular Design, Ltd., (San Leandro, Calif.), Tripos Associates, Inc. (St. Louis, Mo.), and Chemical Abstracts Service (Columbus, Ohio).

De novo design programs include Ludi (Biosym Technologies Inc., San Diego, Calif.), Leapfrog (Tripos Associates, Inc.), Aladdin (Daylight Chemical Information Systems, Irvine, Calif.), and LigBuilder (Peking University, China).

Mimetics, such as peptido- and organomimetics can be designed to fit, e.g., a peptide binding site with current computer modeling software (using computer assisted drug design or CADD) (Walters, 1993; Munson, 1995). Also included within the scope of the disclosure are mimetics prepared using such techniques. In one example, a mimetic mimics a peptide or region of SOCS-3.

Mimetics can be generated using software that can derive a virtual peptide model from several peptide structures. This can be done using the software derived from SLATE algorithm (Perkin et al. (1995), Mills et al. (2001), De Esch et al. (2001), Mills et al. (1997)).

Other approaches to designing peptide analogs, derivatives and mimetics are also well known in the art, see for example Farmer (1980), Ball and Alewood (1990), Morgan and Gainor (1989), Freidinger (1989), Sawyer (1995), Smith et al. (1995), Smith et al. (1994) and Hirschman et al. (1993).

If required, the prospective drug (agonist or antagonist) can be synthesized or obtained from a suitable source such as a commercial supplier. It can then be placed into any standard binding assay to test its effect on the ability to bind a C6orf106 protein, and/or placed in a standard assay to determine its ability to modulate the biological activity of a C6orf106 protein or the ability to modulate virus infection.

For all of the drug screening assays described herein further refinements to the structure of the drug will generally be necessary and can be made by the successive iterations of any and/or all of the steps provided by the particular drug screening assay.

### Administration

The method of the present invention includes administering a compound or composition which modifies C6orf106 protein activity to the subject by an appropriate route, either alone or in combination with another compound such as an antigen which stimulates an immune response.

A variety of routes of administration are possible including, but not limited to, oral, dietary, topical, parenteral (e.g., intravenous, intra-arterial, intramuscular, intradermal, intravascular or subcutaneous injection), and inhalation (e.g., intrabronchial, intranasal or oral inhalation, intranasal drops) routes of administration.

In one embodiment, the compound or composition is administered to a mucosal site. Examples of mucosal sites, include but are not limited to the respiratory tract such as the nasal region (e.g., the nose), the trachea, bronchi and the lungs, the buccal, or oral tissues including the oral (e.g., the mouth and gingivae) and oro-pharyngeal cavities, the throat including the tonsils, the conjunctiva of the eyes, the gastrointestinal tract (e.g., oesophagus, stomach, duodenum, small and large intestines, colon and rectum), the reproductive tract/tissues (including but is not limited to the bladder, ureter, urethra and associated tissues, the penis, the vulva/vagina and cervico-vaginal tissues, as well as the uterus and fallopian tubes).

Formulation of the composition to be administered will vary according to the route of administration selected (e.g., solution, emulsion, capsule).

A composition comprising the compound may contain a physiologically acceptable carrier. For solutions or emulsions, suitable carriers include aqueous or alcoholic/aqueous solutions, emulsions or suspensions, including saline and buffered media. Parenteral carriers include sodium chloride solution, Ringer's dextrose, dextrose and sodium chloride, lactated Ringer's or fixed oils. Intravenous carriers include various additives, preservatives, or fluid, nutrient or electrolyte replenishers and the like. For inhalation, a soluble composition can be loaded into a suitable dispenser for administration (e.g., an atomizer, nebulizer or pressurized aerosol dispenser).

### EXAMPLES

### Example 1

### Materials and Methods

Cells and Viruses: HeLa cells (ATCC CCL-2) and Vero cells (ATCC CRL-81) were maintained in growth medium (Eagles Modified Eagle Medium [EMEM] supplemented with 10% v/v fetal bovine serum [FBS], 10 mM HEPES, 2 mM L-glutamine and 100 U/ml penicillin, and 100 μg/ml streptomycin [P/S; Life Technologies]). A549 cells (ATCC CCL-185) were maintained in Dulbeccol's Modified Eagle Medium/Nutrient Mixture F-12 (DMEM-F12) supplemented with FBS, L-glutamine and P/S as above. All cells were kept at 37° C. in a humidified incubator (5% CO₂). Strains of virus used were as follows: Hendra Virus (HeV) (Hendra virus/Australia/Horse/1994/Hendra); Influenza A virus (A/chicken/Vietnam/008/2004 H5N1); Mumps virus; Respiratory Syncytial Virus (RSV); Vesicular Stomatitis Virus (VSV/Atlanta/Bull/1962); West Nile Virus (WNV NY99); (WNV/New York/Crow/1999).

TCID₅₀ analysis: For 50% tissue culture infective dose (TCID₅₀) analysis, 10-fold dilutions of tissue culture supernatants were made in medium and Vero cells added (5×10⁴ cells/well) in a 96-well tissue culture. Plates were incubated for 3 days (HeV, VSV) or 6 days (WNV, H5N1, MuV) at 37° C. and 5% CO₂ and scored for cytopathic effect. The infectious titer was calculated by the method of Reed and Muench (1938).

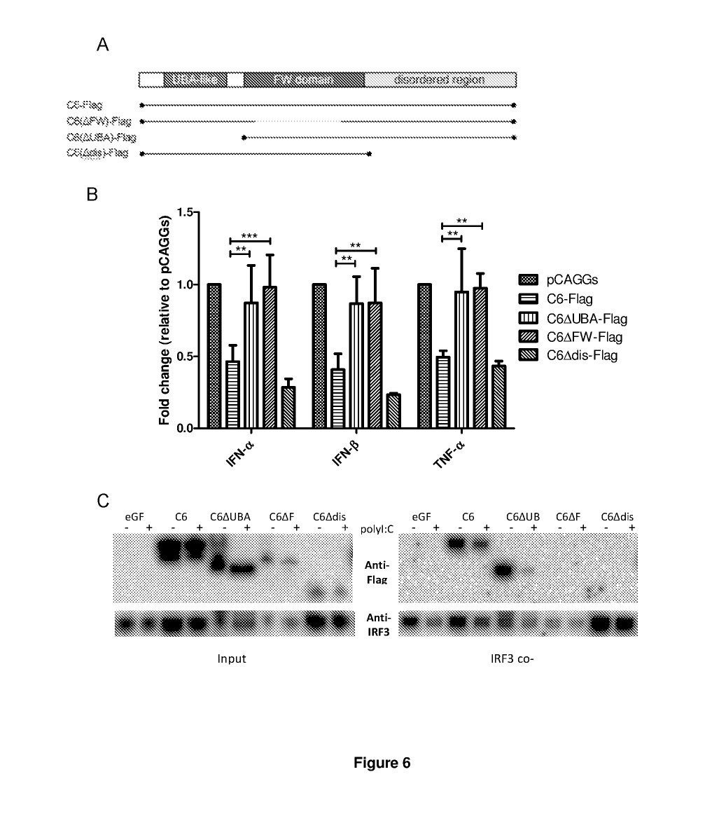

C6orf106 deletion mutant generation: Amplicons with specific deletions (see FIG. 6) were generated with Q5 high-fidelity DNA polymerase (New England Biolabs; NEB) using C6orf106 specific primers (Table 2). Following cleanup and restriction digest (SacI and XhoI; NEB), products were ligated into the pCAGGs mammalian expression vector and transformed into chemically competent E. coli (Max efficiency DH5 competent cells; Life Technologies). Plasmids were amplified and extracted using QIAGEN EndoFree Plasmid Maxi kit as specified by the manufacturers.

TABLE 2
List of primers used in the generation of C6orf106 deletion mutants

| Primer name | Sequence (5′-3′) | SEQ ID NO |
|---|---|---|
| SacI-START-C6 For | ATTGAGCTCGCCACCATGGAAGGAATG | 14 |
| XhoI-C6-FLAG Rev | TAGCTCGAGTCATCATTTGTCGTCGTCAT | 15 |
| | C | |
| XhoI-C6(1-276)-FLAG | TTCTCGAGTCACTTGTCATCGTCATCCTT | 16 |
| Rev | GTAATCTCCATGACTAGAGGGGCTC | |
| XhoI-C6(1-193)-FLAG | TTCTCGAGTCACTTGTCATCGTCATCCTT | 17 |
| Rev | GTAATCTCCGCTGCTCAGCTGCTGG | |
| XhoI-C6(1-76)-FLAG | TTCTCGAGTCACTTGTCATCGTCATCCTT | 18 |
| Rev | GTAATCTCCGCTCATTGATGGCAC | |
| SacI-START-C6-UBA | ATTGAGCTCGCCACCATGAGCTTTGTGGA | 19 |
| For | AGAC | |

Transfections: HeLa cells (7×10⁴) were reverse-transfected with 50 nM siRNA pools (GE Life Sciences; siGENOME human C6orf106 SMARTpool M-016330-01-0050 comprising: the target sequences D-016330-02-ACACACAGCCGCAUCGUAA, D-016330-03-GAGUCAAUACCUCCGGAUA, D-016330-04-GGGUGGACUUUUAGGAGUA, D-016330-17-CAGCAAUUGGCGCCUAUUA) using 0.5 μL Dharmafect-1 (GE Life Sciences) in Opti-MEM (Life Technologies) overnight, after which media was removed and replaced with transfection media (growth media minus antibiotics) and cells incubated for a further 24 hours. For DNA transfections, 300 ng DNA was incubated with 1 μL Lipofectamine 2000 in Opti-MEM (Life Technologies) and used to reverse-transfect HeLa cells (1.4×10⁵/well). Media was replaced ˜6 hours post transfection (h.p.t.) and incubated for a further 18 hours. Cells were stimulated with transfected high molecular weight poly(I:C) (Invivogen) (5-10 μg/mL with 1.5 μL Lipofectamine 2000) for 6 hours in transfection media as above.

RNA purification, reverse transcription and quantitative real-time PCR (qRT-PCR): Cells were lysed in 500 μL Trizol (Life Technologies) or Trisure (Bioline) and RNA extracted according to manufacturer's protocols. Following DNAse treatment (RQ1 DNase, Promega), 500 ng RNA was reverse-transcribed to DNA with oligo-d(T) and random hexamers using Superscript III RT (Life Technologies) or Sensifast RT (Bioline) first strand cDNA synthesis protocols. qRT-PCR was performed using Sybr green (Applied Biosystems, Foster City, Calif.) on a StepOne Plus PCR cycler (Applied Biosystems). PCR cycling for gene detection was at 95° C. for 10 min, followed by 45 cycles of 95° C. for 15 s and 60° C. for 1 min. A melting curve analysis was performed to eliminate primer-dimer artifacts and to verify the specificity of the assay. Cytokine expression and virus RNA transcription data were analyzed using the ΔΔC_{T} method and were normalized to glyceraldehyde-3-phosphate dehydrogenase (GAPDH) for gene detection. Primers used in qRT-PCR analyses are shown in Table 3.

TABLE 3
List of primers used in qRT-PCR analyses

| | | SEQ ID |
|---|---|---|
| Primer name | Sequence (5′-3′) | NO |
| IFN-α For | CCGTGAGTTTCCCAGAAGAA | 20 |
| IFN-α Rev | ACTGCCCAAGATGAAGACCA | 21 |
| IFN-β For | AGTAGGCGACACTGTTCGCA | 22 |
| IFN-β Rev | AGCCTCCCATTCAATTGCCA | 23 |
| IL6 For | ACCCCCAGGAGAAGATTCCA | 24 |
| IL6 Rev | CACCAGGCAAGTCTCCTCATT | 25 |
| TNF-α For | CAACCTCCTCTCTGCCATCAAGA | 26 |
| TNF-α Rev | CTGGAAGACCCCTCCCAGATAGA | 27 |
| ISG15 For | CAGCCATGGGCTGGGAC | 28 |
| ISG15 Rev | TCCTCACCAGGATGCTCAGA | 29 |
| IκBα For | GCAAAATCCTGACCTGGTGT | 30 |
| IκBα Rev | GCTCGTCCTCTGTGAACTCC | 31 |
| huC6orf106 | TGGGTGATTCTCAGTGTGGAGG | 32 |
| For | | |
| huC6orf106 | TCTACCTTACGATGCGGCTGTG | 33 |
| Rev | | |
| GAPDH For | CTATAAATTGAGCCCGCAGCC | 34 |
| GAPDH Rev | ACCAAATCGGTTGACTCCGA | 35 |

Dual-luciferase assays: HeLa cells were reverse-transfected with 100 ng eGFP/C6-FLAG overexpression plasmids, in conjunction with 100 ng of either ISRE-luciferase or NF-κB-luciferase (Firefly) and 50 ng of a Renilla luciferase construct. At 20 h.p.t, cells were stimulated with poly(I:C) as indicated above for 6 hours, then lysed in Passive Lysis Buffer (PLB; Promega). Lysates were then assayed for successive Firefly and Renilla luciferase activity using the a dual-luciferase kit (Promega) as per manufacturer's recommendations.

Immunoprecipitation and cell fractionation: For immunoprecipitation, 25 μg of antibody was coupled to 50 μg of agarose beads using the Pierce direct immunoprecipitation kit as specified by the manufacturers (Life Technologies). Cells were lysed in NP-40 lysis buffer provided and pre-cleared with a non-specific isotype agarose control at 4° C. for 1 hour, then incubated with ˜1.5 μg agarose-coupled antibody at 4° C. overnight. Resin was washed multiple times then elution performed using non-reducing sample buffer (containing 2% w/v SDS) at 100° C. for 10 mins. Cytosolic and nuclear fractions were prepared from cells using the Pierce NE-PER cell fractionation kit (Life Technologies) as specified by the manufacturers, and total protein quantitated by BCA assay (Life Technologies) before western blot analyses.

Western blotting: Cells were lysed in SDS lysis buffer (50 mM Tris-HCl pH 8.0, 2 mM EDTA pH 8.0, 150 mM NaCl, 0.5% w/v SDS and 10% glycerol) or LDS sample buffer (Life Technologies) supplemented with a protease/phosphatase inhibitor cocktail (Astral Scientific). Protein lysates were separated on a 4-12% gradient NuPAGE polyacrylamide gel in LDS sample buffer (Life Technologies) at 120V, then transferred to a nitrocellulose membrane using the TransBlot system (Bio-Rad). Following blocking in 3% skim milk powder/TBS (+0.05% Tween-20), membranes were incubated with primary antibodies in blocking solution at 4° C. overnight. HRP-conjugated secondary antibodies were diluted 1:5000 in blocking solution and incubated for 3 hours at RT with blot. Membranes were washed and rinsed in TBS-Tween, and incubated with ECL-developing solution (Bio-Rad) and detected on Chemi-Doc (Bio-Rad).

Immunofluorescence: Cells were fixed in 4% w/v paraformaldehyde in PBS at RT for 20 mins, followed by permeabilisation with 0.1% v/v Triton X-100 and quenching with 0.2M Glycine for 10 mins each at RT. Fixed cells were blocked in 1% bovine serum albumin (BSA; Fraction V, Sigma-Aldrich) in PBS for 30 min, then incubated with primary antibodies in blocking solution for 1 hour at RT, washed 3 times in 0.2% BSA/PBS, and incubated with secondary antibodies conjugated to either Alexa Fluor 488 or Alexa Fluor 568 (Life Technologies) in 1% BSA/PBS at RT for 1 hour. Cells were washed in PBS then counterstained with the nuclear dye DAPI (0.5 μg/mL) and viewed on a Leica SP5 confocal microscope or analysed on the CellInsight.

IFN-β ELISA: Cell culture supernatants from HeLa cells transfected then stimulated with poly(I:C) were collected and analysed for IFN-β secretion using a sandwich ELISA kit as supplied by Elisakit.com according to manufacturer's protocols. IFN-β concentrations were calculated by comparison to standards using a polynomial regression method.

### Example 2

### C6orf106 is Required for Virus Infection

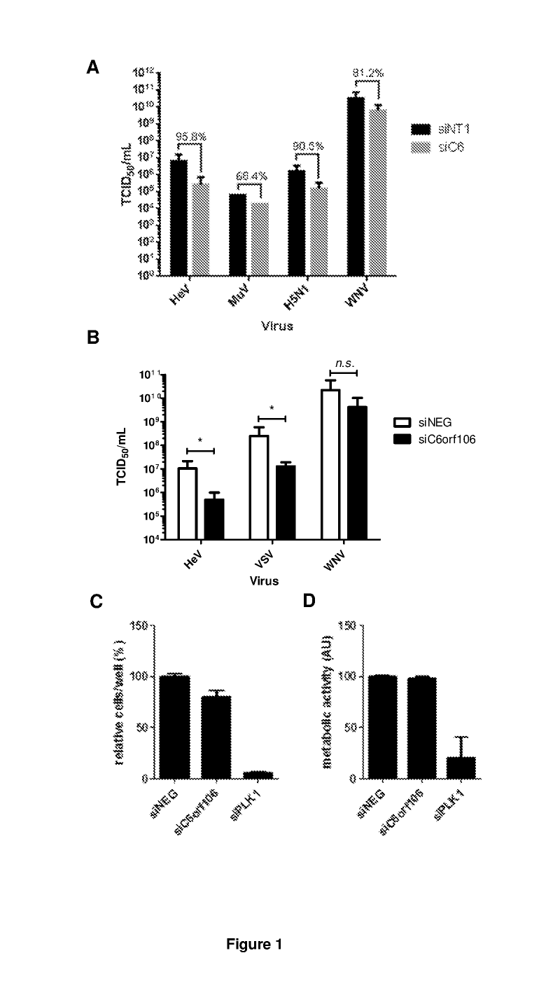

C6orf106 was identified in an siRNA screen investigating proteins (host proteins) required for Hendra virus (HeV) infection. Knockdown of C6orf106 resulted in a significant decrease in HeV and NiV infectious virus production. To validate the screen result for C6orf106, HeLa cells were transfected with siRNAs targeting C6orf106 (50 nM) 48 hours, then infected with negative-strand RNA viruses for 24 hours, after which virus production was assayed by TCID₅₀ in Vero cells. Cell lysates collected from HeLa cells transfected with a SMARTpool siC6orf106 siRNA showed a 90% decrease in C6orf106 expression levels at both the mRNA and protein level compared to cells transfected with siNEG, a negative control SMARTpool siRNA that does not target any gene. As shown in FIGS. 1A and B, siC6 knockdown reduced virus production in HeLa cells by approximately 1.5 logs (˜95%) compared to the negative, non-targeting control (siNT1). These experiments were repeated with infection of the cells with a paramyxovirus (Mumps virus; MuV), Vesicular stomatitis virus (VSV, Rhabdoviridae), an orthomyxovirus (Influenza A strain H5N1) or a positive sense RNA virus (West Nile Virus strain NY99; WNVNY99). MuV, H5N1 and WNVNY99 infection were also affected by C6orf106 knockdown. A significant reduction HeV (Paramyxoviridae), Vesicular stomatitis virus (VSV, Rhabdoviridae) and Influenza A (H5N1) virus (Orthomyxoviridae) virus titres were observed in supernatant collected 24 h after infection (Figure A and B). By contrast, siC6orf106 had no significant impact on WNVNY99 (Flaviviridae) titres, suggesting that C6orf106 promotes infection by negative-strand RNA viruses.

To assess the impact of C6orf106 knockdown on cell health HeLa cells were transfected with SMARTpool siC6orf106 or an siNEG control and cell numbers were assessed 72 hr post-transfection. A significant difference in cell numbers was not observed with siC6orf106 silencing 72 h post transfection (FIG. 1C). An Alamar blue assay showed no significant change in metabolic activity in cells treated with the siC6orf106 SMARTpool compared to siNEG (FIG. 1D). As a positive control for cell death, cells were transfected with a SMARTpool siRNA targeting polo-like kinase 1 (PLK1), a gene associated with apoptosis induction (Liu et al., 2003). Decreases in both cell number and cell viability were observed in cells transfected with siPLK1 (FIGS. 1D and E).

### Example 3

### C6orf106 is Highly Conserved Amongst Vertebrate Species with Two Putative Functional Domains

The C6orf106 amino acid sequence was sourced from NCBI (Accession number Q9H6K1.2) and aligned with homologs extracted from the Ensembl database (http://www.ensembl.org/index.html) using ClustalW software. As shown in FIG. 2, the C6orf106 protein sequence is highly conserved across the vertebrate species, with 50% of residues identical from humans to the sea squirt (C. intestinalis) sequence. The highest degree of conservation is evident in the two putative functional domains; the ubiquitin-associated-like (UBA-like) domain, and the Nbr-1-like or FW domain, which is characterised by four tryptophan residues (see FIG. 2). This latter domain in the ubiquitin receptor Nbr-1 has been shown to facilitate interactions with the microtubule associated protein MAP1B and potential links between the autophagy pathway and the microtubule network (Marchbanks et al., 2012). Mining of the ClustalW database suggests that C6orf106 is the only human protein identified to date featuring a UBA-like domain and an FW domain.

### Example 4

### C6orf106 Knockdown Enhances Cytokine Transcription in Response to Poly(I:C)

The effect of C6orf106 silencing on cytokine transcription was next assessed. A synthetic double-stranded RNA analog Polyinosinic-polycytidylic acid (poly(I:C)) was used to mimic viral RNA replication and the induction of interferons and inflammatory cytokine transcription. HeLa cells were treated with C6orf106 siRNAs for 48 hours as described above, and then stimulated with transfected poly(I:C) for 6 hours, after which RNA was extracted and analysed for the transcription of interferon alpha and beta (IFN-α/β) as well as the pro-inflammatory cytokines interleukin-6 (IL-6) and tumor necrosis factor alpha (TNF-α).

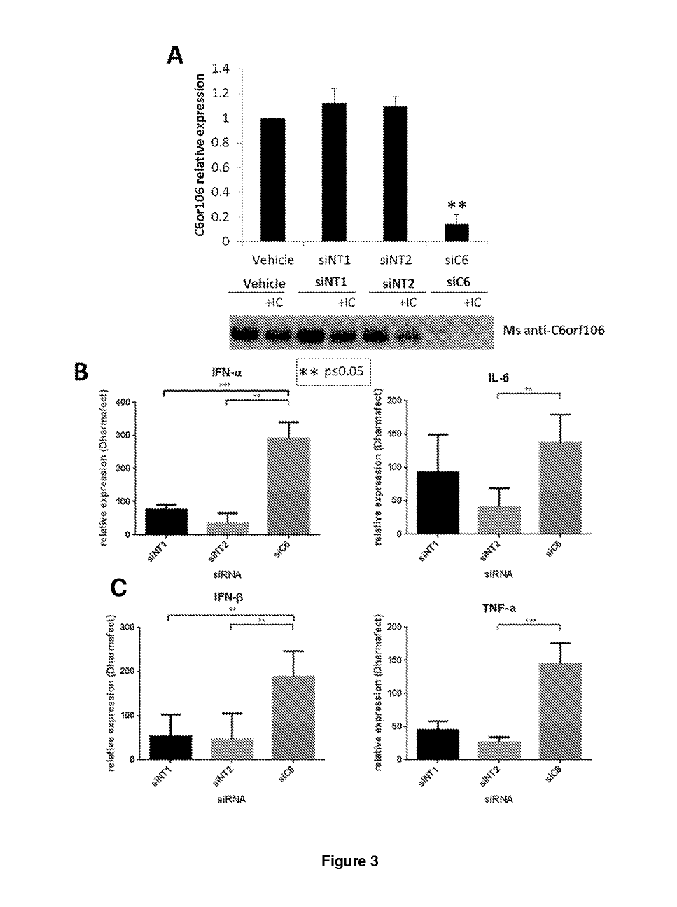

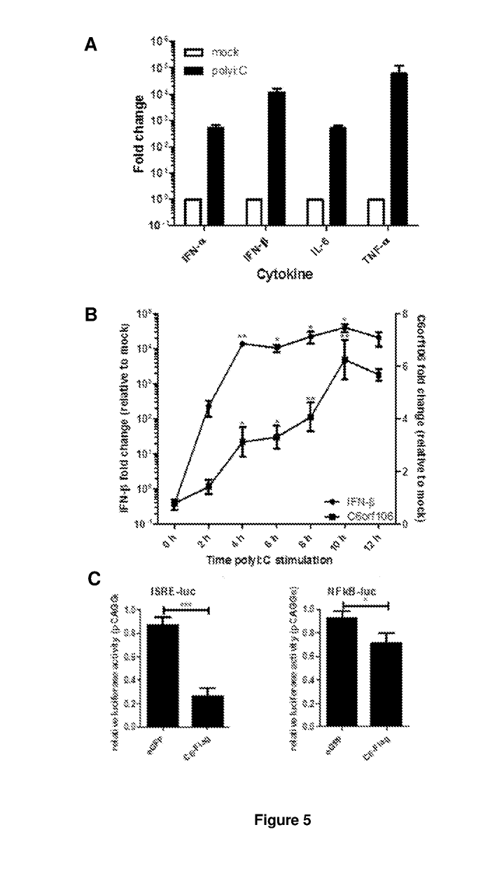

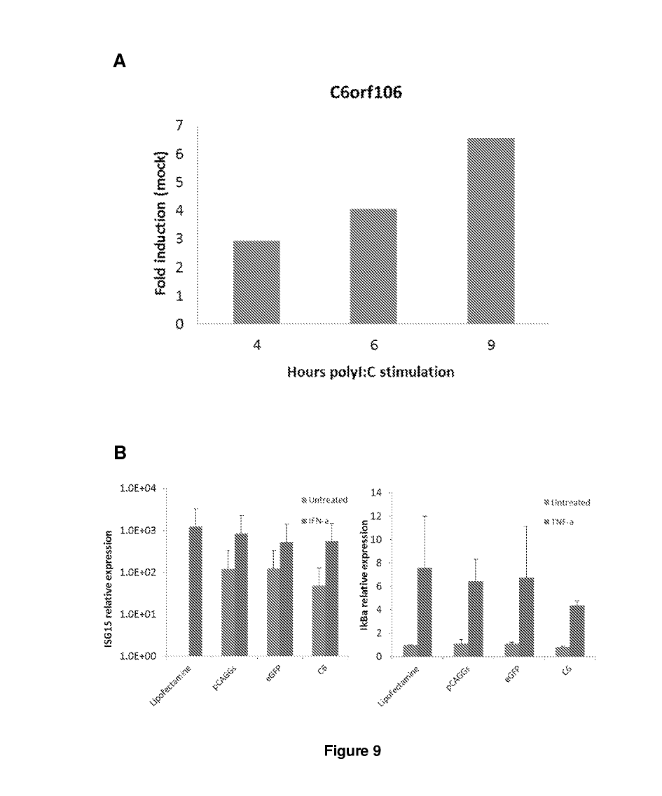

FIG. 3A shows the successful knockdown of C6orf106 at the RNA level (˜90% reduction as assessed by qRT-PCR) and nearly-undetectable protein using a C6orf106-specific antibody. In the non-targeting controls (siNT1/NT2) stimulated with poly(I:C), transcription of IFN-α, IFN-β, IL-6 and TNF-α all increased compared to the unstimulated cells. HeLa cells treated with intracellular poly(I:C) showed a robust up-regulation of IFN-α/β, interleukin-6 (IL-6) and (TNF-α) (FIGS. 5A and B). Transcriptional induction of IFNα/β and TNF-α was significantly enhanced in the C6orf106-knockdown cells, however IL-6 transcription was not reproducibly changed (FIG. 3B). This suggests that C6orf106 acts as a negative regulator of poly(I:C) induced antiviral signalling. This is further supported by the observation that endogenous C6orf106 RNA levels increase with time after poly(I:C) stimulation (FIG. 9A) as would be predicted by a negative feedback loop.

### Example 5

### C6orf106 Overexpression Inhibits Interferon α/β Transcription and Secretion in Response to Poly(I:C)

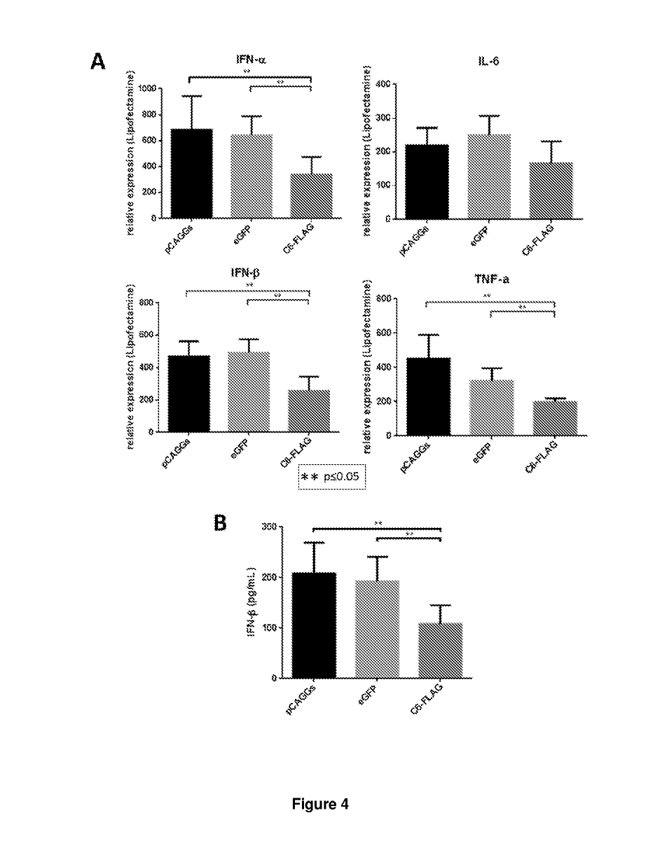

To confirm the potential role for C6orf106 as a negative regulator of poly(I:C)-induced IFN signalling. HeLa cells were transfected with a C6orf106 expression vector (pCAGGs-C6-FLAG; SEQ ID NO: 40) as well as a non-specific GFP over-expression control. At 18 h post-transfection, cells were stimulated with poly(I:C) for 6 hours, and extracted RNA analysed for interferons and inflammatory cytokines as above. GFP or the vector alone (pCAGGs) did not impair cytokine transcription in response to poly(I:C) (as shown in FIG. 4A), however C6orf106 over-expression resulted in a significant reduction in IFN-α, IFN-β and TNF-α transcription. Induction of IL-6 transcripts were unaffected by C6orf106. Furthermore, when tissue culture supernatants were assessed for the secretion of IFN-β by ELISA, a significant decrease in IFN-β concentration in cells over-expressing C6orf106 was observed. Given that the relative reduction in IFN-β mRNA and protein levels were similar, this was attributed to a C6-mediated decrease to reduced mRNA/protein expression, in contrast to a block in the secretory pathway. Interestingly, downstream IFN and/or TNF-α signalling was not significantly affected by expression of C6orf106 (FIG. 9B), as shown by unchanged ISG15 or IκBα transcription in response to IFN-α or TNF-α treatment respectively.

The impact of C6orf106 expression on the activity of the individual transcription factors was assessed using luciferase vectors containing IRF3 and/or NF-κB binding sites. In response to intracellular poly(I:C), C6orf106 over-expression resulted in a ˜75% reduction of ISRE-luciferase activity compared to cells transfected with GFP (FIG. 5C). A modest reduction in NF-κB-luciferase was also observed.

### Example 6

### Deletion of the UBA-Like Domain of C6orf106 Enhances its Transcriptional Inhibitory Effect

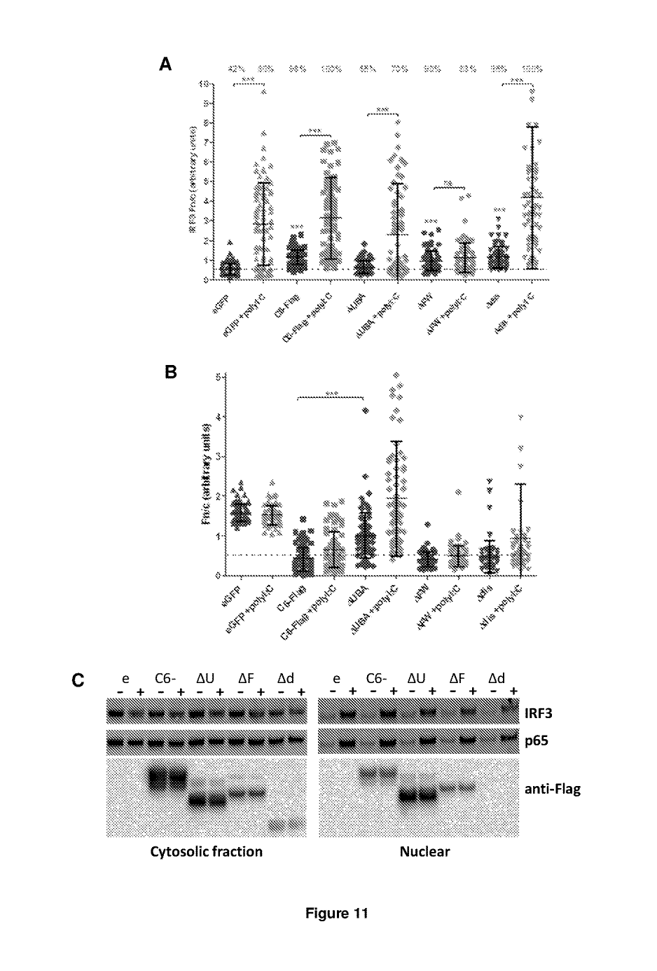

To determine which of the putative functional domains of C6orf106 were responsible for its impact on cytokine transcription. Expression plasmids were generated by deleting the UBA-like domain (ΔUBA) SEQ ID NO: 44, the FW/Nbr-1-like domain (ΔFW) SEQ ID NO: 49 and the disordered region (Δdis) SEQ ID NO: 42 as shown in FIG. 6A. As shown in FIG. 6B, deletion of either the UBA-like or the FW domain resulted in significantly higher levels of IFNα/β and TNF-α mRNA in response to poly(I:C) when compared to full-length C6orf106-FLAG. In contrast, removal of the disordered region did not restore cytokine transcription and in the case of IFNα/β actually reduced these levels further. This was observed despite lower overall levels of the Δdis protein itself compared to C6orf106-FLAG and the other deletion mutants (FIG. 11). Intriguingly, subcellular localisation of the Δdis protein most closely resembled full-length C6orf106-FLAG (FIG. 11), and similar to full-length C6orf106, did not impair IRF3 nuclear translocation in response to poly(I:C). By comparison, both the ΔUBA and ΔFW proteins displayed altered subcellular distributions, with ΔUBA being retained in the nucleus whilst ΔFW was more cytoplasmic.

### Example 7

### C6orf106 does not Impair Nuclear Translocation of Transcription Factors in Response to Poly(I:C)

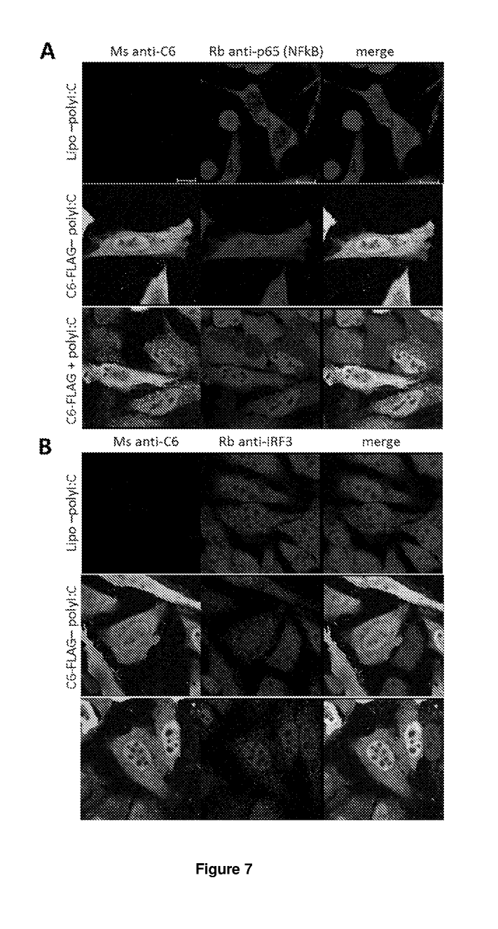

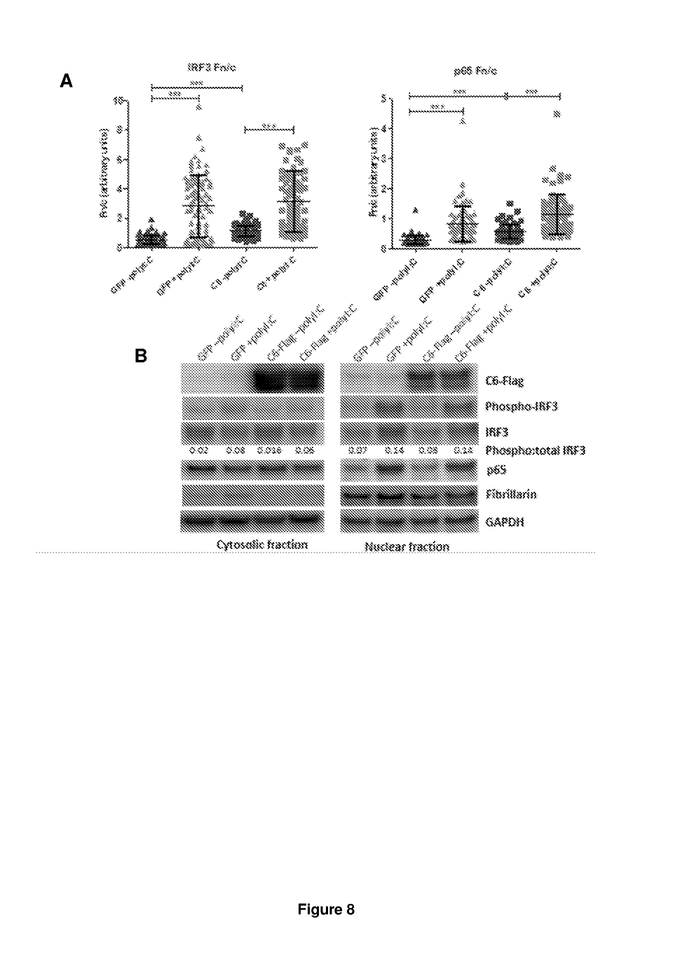

IFN-α/β transcription is primarily controlled by interferon response factor 3 (IRF3) as well as nuclear factor κ-B (NFκB), both of which translocate to the nucleus in response to TLR and RLR ligands, such as poly(I:C). To investigate the mechanism by which C6orf106 inhibits cytokine transcription C6orf106 was expressed in HeLa cells stimulated with poly(I:C) as described above, then fixed cells and labelled for C6orf106-Flag, IRF3 or p65 (NFκB). Neither IRF3 nor p65 nuclear localisation was inhibited in C6orf106-expressing cells (FIGS. 7A, 7B and FIG. 8A) that were stimulated with poly(I:C). However, nuclear staining of C6orf106-Flag was observed which increased upon poly(I:C) stimulation, suggesting that C6orf106 may exert its effects on transcription at the nuclear level, rather than upstream signalling events. There was also a modest, but significant, increase in IRF3 and p65 nuclear staining in unstimulated cells expressing C6orf106 compared to GFP.

Following RLR/TLR activation by ligands such as poly(I:C), a cascade of signalling effectors result in IRF3 phosphorylation, dimerization and nuclear trafficking. To assess whether C6orf106 blocks IRF3 activation upon recognition of viral RNA-like stimuli, the nuclear and cytosolic fractions of GFP and C6orf106 expressing cells were isolated and probed for phosphorylated IRF3 (Ser396). Poly(I:C) stimulation increased levels of phosphorylated IRF3 in both GFP- and C6orf106-expressing cells (FIG. 8B). Additionally, nuclear accumulation of p65, phospho-IRF3 and total IRF3 was observed to equal extents in GFP- and C6orf106-expressing cells. C6orf106 inhibits IRF3/p65-mediated transcription downstream of transcription factor activation and nuclear translocation.

### Example 8

### C6orf106 Binds to IRF3 and this Binding is Enhanced in Response to Poly(I:C)

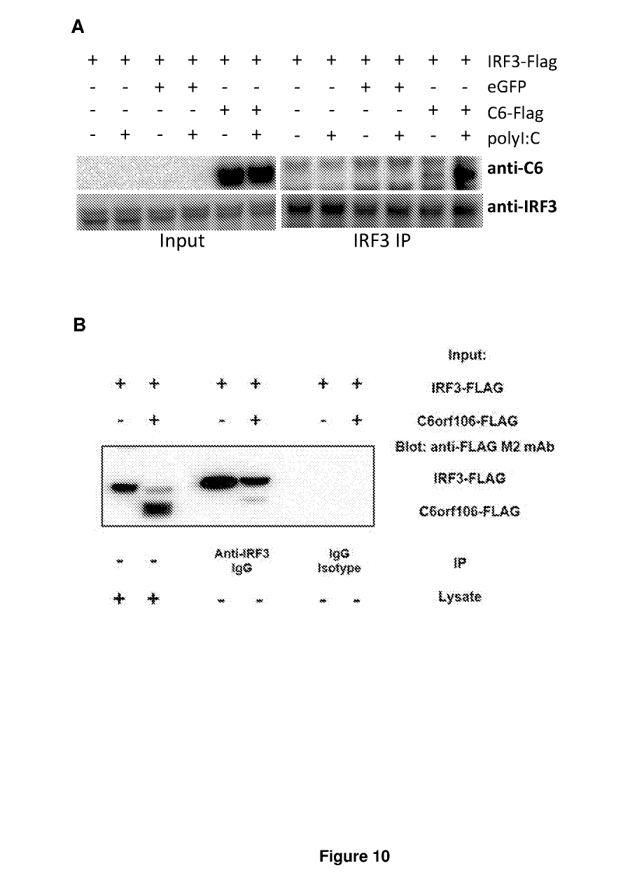

To access whether C6orf106 binds IRF3, C6orf106-flag was co-expressed with GFP −/+ poly(I:C) as described above, then performed a direct co-immunoprecipitation with immobilised IRF3 antibodies. As shown in FIG. 10A C6orf106 could be detected in IRF3-immunoprecipitated lysates, and this was increased approximately four-fold in the presence of poly(I:C). Further, to show that C6orf106 binds to IRF3 HEK293T cells were transfected with IRF3 alone, or IRF3 in combination with C6orf106. Cells were lysed and subjected to indirect immunoprecipitation with an anti-IRF3 antibody. IP samples and input controls were probed with anti-FLAG antibody for western blotting. An IgG isotype was used as a negative control for the immunoprecipitation experiment. The results, as shown in FIG. 10B, demonstrate that C6orf106 binds to IRF3.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

This application claims priority from Australian Provisional Application No. 2015905035 entitled “Regulation of cytokine production” filed on 4 Dec. 2015, the entire contents of which are hereby incorporated by reference.

All publications discussed and/or referenced herein are incorporated herein in their entirety.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

### REFERENCES

Bird et al. (1988) Science 242:423-426.
Ball and Alewood (1990) J. Mol. Recognition 3:55.
Bohm and Stahl (1999) M. Med. Chem. Res. 9:445.
Cadwell and Joyce (1992) PCR Methods Appl. 2:28-33.
Cerqueira et al. (2015) Arch Biochem Biophys 582:56-67.
Coco et al. (2001) Nature Biotechnology 19:354-359.
Coco et al. (2002) Nature Biotechnology 20:1246-1250.
Constantini et al. (2008) Cancer Biotherm Radiopharm. 23:3-24.
Crameri et al. (1998) Nature 391:288-291.
Daya et al. (2008) Clin. Microbiol Rev. 21(4):583-593.
De Esch et al. (2001) J. Med. Chem. 44:1666-1674.
De Coupade et al. (2005) Biochem J. 390:407-418.
Deshayes et al. (2008) Adv. Drug Deliv. Rev. 60:537-547.
Eggert et al. (2005) Chemobiochem. 6:1062-1067.
Ewing et al. (2001) J. Comput Aid. Mol. Design 15:411.
Farmer, in Drug Design, E. J. Ariens, ed., Academic Press, New York, 1980, v. 10, pp. 119-143.
Freidinger (1989) Trends Pharmacol. Sci. 10:270.
Gane and Dean (2000) Curr. Opinion Struct. Biol. 10:401.
Good (2001) Current Opinion in Drug Disc. Devel. 5, 301.
Goodford (1984) J. Med. Chem. 27:557.
Goodford (1985) J. Med. Chem. 28:849.
Harayama (1998) Trends Biotechnol. 16:76-82.
Harmsen and De Haard (2007) Appl. Microbiol. Biotechnol. 77:13-22.
Hirschman et al. (1993) J. Am. Chem. Soc. 115:12550-12568.
Hol (1986) Angew. Chem. 25: 767.
Howl et al. (2007) Biochem. Soc. Trans. 35:767-769.
Huston et al. (1988) Proc Natl Acad Sci. USA. 85:5879-5883.
Jiang et al. (2015) Tumour Biol. 36:7881-7889.
Kabadi et al. (2015) Nucleic acid Res. 42:e147.
Koppelhus et al. (2008) Bioconj. Chem. 19:1526-1534.
Kuenemann et al. (2015) Prog. Biophys. Mol. Biol 119:20-32.
Kuntz et al. (1982) J. Mol. Biol. 161:269.
Langer and Hoffmann (2001) Current Pharmaceutical Design 7:509.
Leung et al. (1989) Technique 1:11-15.
Liu et al. (2003) (Plk) Proc Natl Acad Sci, 100(10):5789-94.
Makarova et al. (2015) Nature Reviews Microbiology 13:1-15.
Marchbanks et al. (2012) Int J Biol 2012:208014. doi: 10.1155/2012/208014. Epub 2012 May 10.
Meyer-Losic et al. (2006) J. Med Chem. 49:6908-6916.
Mills et al. (2001) J Comp. Aided Mol Des 15:81-96,
Mills et al. (1997) J Comp. Aided Mol Des 11:175-192.
Morgan and Gainor (1989) Ann. Rep. Med. Chem. 24:243.
Morrison et al. (1984) Proc. Natl. Acad. Sci. USA 81:6851-6855.
Mungall et al. (2003) Nature 425: 805-811.
Munson (ed.), Principles of Pharmacology, 1995, Chapman & Hall, Chapter 12.
Muyldermans (2001) J. Biotechnol, 74:277-302.
Needleman and Wunsch (1970) J. Mol. Biol. 48:443-453.
Ness et al. (2002) Nature Biotechnology 20:1251-1255.
Ostermeier et al. (1999) Nat Biotechnol. 17:1205-1209.
Perkin et al. (1995) J Comp. Aided. Mol. Des. 9:479-490.
Pietersz et al. (2006) Current Medicinal Chemistry 13:1591.
Prasher et al. (1985) Biochem. Biophys. Res. Commun. 127:31-36.
Rarey et al. (1996) J. Mol. Biol. 261: 470.
Reed and Muench (1938) The American Journal of Hygiene 27:493-497.
Sawyer (1995) “Peptidomimetic design and chemical approaches to peptide metabolism”, in M D Taylor and G L Amidon, eds., in Peptide-Based Drug Design: Controlling Transport and Metabolism, Ch. 17.
Sheridan and Venkataraghavan (1987) Acc. Chem Res. 20:322.
Sieber et al. (2001) Nature Biotechnology 19:456-460.
Smith et al. (1994) J. Am. Chem. Soc. 116:9947-9962.
Smith et al. (1995) J. Am. Chem. Soc. 117:11113-11123.
Stemmer (1994a) Proc. Natl. Acad. Sci. USA 91:10747-10751.
Stemmer (1994b) Nature 370 (6488):389-391.
Sutcliffe et al. (1983) Science 219:660.
Thillet et al. (1988) Journal of Biological Chemistry 263:12500-12508.
Tibary et al. (2007) Soc. Reprod. Fertil. Suppl. 64:297-313.
Visintin et al. (2008) J. Biotechnol. 135:1-15.
Verlinde and Hol (1984) Structure 2:577.
Volkov et al. (1999) Nucleic Acids Research 27:e18i-vi.
Walters, “Computer-Assisted Modeling of Drugs”, in Klegerman & Groves (eds.), Pharmaceutical Biotechnology, 1993, Interpharm Press: Buffalo Grove, Ill., pp. 165-174.
Walters et al. (1998) Drug Discovery Today 3:160.
Westermaier et al. (2015) Methods 71:44057.
Zetsche et al. (2015) Cell 163:1-3.
Zhang et al. (2011) Nature Biotechnology 29:149-153.
Zhang et al. (2015) J. Chem. Inf. Model 54:324-37.
Zhang et al. (2015) Tumour Biol. 36:5979-5985.
Zhao et al. (1998) Nature Biotechnology 16:258261.

1. A method of modulating an immune response and/or cytokine production in a subject, the method comprising administering to the subject a compound which modifies C6orf106 protein activity.

2. The method of claim 1, wherein the compound increases C6orf106 protein activity, and wherein the immune response and/or cytokine production is reduced.

3. The method of claim 2, wherein increased C6orf106 protein activity reduces IRF3-dependent cytokine transcription.

4. The method of claim 2 or claim 3, wherein the compound is a polynucleotide, a polypeptide or a small molecule.

5. The method of claim 4, wherein the polynucleotide encodes a polypeptide which comprises an amino acid sequence which is at least 50% identical to any one or more of SEQ ID NO's 1 to 11 or a biologically active fragment thereof.

6. The method of claim 5, wherein the polynucleotide is operably linked to a promoter which directs expression of the polynucleotide in the subject.

7. The method of claim 6, wherein the polynucleotide is administered in an expression vector.

8. The method of claim 7, wherein the vector is a viral vector.

9. The method of claim 4, wherein the polypeptide comprises an amino acid sequence which is at least 50% identical to any one or more of SEQ ID NO's 1 to 11 or a biologically active fragment thereof.

10. The method of claim 9, wherein the biologically active fragment lacks a functional disordered region.

11. The method of claim 1, wherein the compound reduces C6orf106 protein activity, and wherein the immune response and/or cytokine production is increased.

12. The method of claim 11, wherein the compound reduces formation of a complex comprising C6orf106 and IRF3.

13. The method of claim 11, wherein reducing C6orf106 protein activity increases IRF3-dependent cytokine transcription.

14. The method of any one of claims 11 to 13, wherein the compound is a polynucleotide, a polypeptide or a small molecule.

15. The method of claim 14, wherein the polynucleotide reduces expression of the C6orf106 gene.

16. The method of claim 14 or claim 15, wherein the polynucleotide is selected from: an antisense polynucleotide, a sense polynucleotide, a polynucleotide which encodes a polypeptide which binds C6orf106, a double stranded RNA molecule or a processed RNA molecule derived therefrom.

17. The method of claim 15 or claim 16, wherein the polynucleotide is expressed from a transgene administered to the subject.

18. The method of claim 14, wherein the polynucleotide binds to C6orf106 and reduces C6orf106 protein activity.

19. The method of claim 18, wherein the polypeptide is an RNA aptamer, a DNA aptamer, or an XNA aptamer.

20. The method of any one of claims 11 to 14, wherein the compound binds to C6orf106 and reduces C6orf106 protein activity.

21. The method of claim 20, wherein the compound is a polypeptide.

22. The method of claim 21, wherein the polypeptide is an antibody or antigen binding fragment.

23. The method according to any one of claims 1 to 22, wherein the immune response is an IFN response.

24. The method of claim 23, wherein the immune response is a type I IFN response.

25. The method according to any one of claims 1 to 24, wherein the cytokine is one, more or all of IFN-α, IFN-β and TNF-α.

26. The method according to any one of claims 1 to 25, wherein the immune response is selected from: at anti-viral immune response, an autoimmune response, an inflammatory response.

27. The method of claim 26, wherein the immune response is an anti-viral immune response and the immune response and/or cytokine production is increased.

28. The method of claim 26, wherein the immune response is an inflammatory response and the immune response and/or cytokine production is reduced.

29. The method according to any one of claims 1 to 26 wherein the subject has one or more of the following conditions: an infection, an immunodeficiency, an autoimmune disease, an inflammatory condition or cancer.

30. The method of claim 29, wherein the infection is a virus infection.

31. The method of claim 30, wherein the virus in a negative-strand RNA virus.

32. The method of claim 30, wherein the virus is selected from a: Orthomyxoviridae, Retroviridae, Herpesviridae, Paramyxoviridae, Rhabdoviridae, Filoviridae, Bornaviriade and Coronaviridae.

33. The method of any one of claims 30 to 32, wherein the subject is also administered with at least one antigen which stimulates an immune response to the virus.

34. The method of claim 29, the autoimmune disease is selected from: Ulcerative colitis, Crohn's disease, Irritable bowel syndrome, Rheumatoid arthritis, Polyarthritis, Multiple sclerosis, Uveitis, asthma, Type 1 diabetes, Type 2 diabetes, Lupus or Chronic obstructive pulmonary disease.

35. The method of claim 29, wherein the subject is also administered with at least one antigen which stimulates an immune response to the cancer.

36. A method of treating and/or preventing an infection, immunodeficiency or cancer in a subject, the method comprising administering to the subject a compound which reduces C6orf106 protein activity.

37. A method of treating and/or preventing autoimmune disease in a subject, the method comprising administering to the subject a compound which increases C6orf106 protein activity.

38. The method according to any one of claims 1 to 37, wherein C6orf106 comprises an amino acid sequence which is at least 50% identical to any one of SEQ ID NO's 1 to 11.

39. The method according to any claims 1 to 38, wherein the subject is an animal.

40. The method of claim 39, who the subject is a mammal.

41. The method of claim 40, wherein the subject is a human.

42. Use of a compound which modifies C6orf106 protein activity in the manufacture of a medicament for modulating an immune response and/or cytokine production in a subject.

43. Use of a compound that reduces C6orf106 protein activity in the manufacture of a medicament for treating an infection, immunodeficiency or cancer in a subject.

44. Use of a compound that increases C6orf106 protein activity in the manufacture of a medicament for treating autoimmune disease in a subject.

45. A compound which modifies C6orf106 protein activity for use in modulating an immune response and/or cytokine production in a subject.

46. A compound which reduces C6orf106 protein activity for use in treatment of a virus infection or cancer.

47. A compound which increases C6orf106 protein activity for use in treatment of an autoimmune disease.

48. A method of identifying a compound which modifies C6orf106 protein activity, the method comprising:
i) contacting a cell with a candidate compound, and
ii) determining whether the compound increases or reduces C6orf106 protein activity in the cell.

49. A method of identifying a compound which modifies C6orf106 protein activity, the method comprising:
i) contacting a cell with a candidate compound, and
ii) determining whether the compound increases or reduces IRF3-dependent cytokine transcription in the cell.

50. A method of identifying a compound which reduces C6orf106 protein activity, the method comprising:
i) contacting a cell with a candidate compound, and
ii) determining whether the compound reduces formation of a complex comprising C6orf106 and IRF3 in the cell.

51. The method of any one of claims 48 to 50 which comprises determining the level of C6orf106 mRNA in the cell.

52. The method of any one of claims 48 to 50 which comprises determining the level of C6orf106 protein the cell.

53. A method of identifying a compound that binds C6orf106, the method comprising:
i) contacting a polypeptide which comprises an amino acid sequence which is at least 50% identical to any one of SEQ ID NO's 1 to 11 or a biologically active fragment thereof, with a candidate compound, and
ii) determining whether the compound binds the polypeptide.

54. The method of claim 53, wherein e candidate compound is an antibody or fragment thereof, an aptamer or a small molecule.

55. A method of identifying a compound which modifies C6orf106 protein activity in silico, the method comprising:
i) generating a three dimensional structural model of a polypeptide comprising an amino acid sequence which is at least 50% identical to any one of SEQ ID NO's 1 to 11 or a biologically active fragment thereof, and
ii) designing or screening for a compound which potentially binds the structure, and/or
iii) designing or screening for a compound that reduces formation of a complex comprising C6orf106 and IRF3.

56. The method of claim 55 which further comprises testing the compound designed or screened for in ii) for its ability to bind C6orf106 and modulate C6orf106 protein activity.

57. The method of claim 55 or claim 56 which further comprises testing the compound designed or screened for in ii) for its ability to modulate virus infection.

58. An isolated and/or recombinant mutant of a naturally occurring C6orf106 polypeptide which has a modified activity compared to the naturally occurring molecule.

59. The isolated and/or recombinant mutant of claim 58 which comprises an amino acid sequence which is at least 50% identical to any one of SEQ ID NO's 1 to 11 but lacks a functional UBA-like domain, a functional disordered region, and/or a functional FW domain.

60. The isolated and/or recombinant mutant of claim 58 or claim 59 which lacks about 76 N-terminal amino acids amino of any one of SEQ ID NO's 1 to 11.

61. An isolated and/or exogenous polynucleotide encoding the isolated and/or recombinant mutant of claim 59 or claim 60.


SEQUENCE LISTING (62 sequences)

SEQ ID NO: 1 - protein, 298 aa, Homo sapiens
MEGMDVDLDPELMQKFSCLGTTDKDVLISEFQRLLGFQLNPAGCAFFLDMTNWNLQAAIGAYYDFESPNISVPSMSFVEDVTIGEGESIPPDTQFVKTWRIQNSGAEAWPPGVCLKYVGGDQFGHVNMVMVRSLEPQEIADVSVQMCSPSRAGMYQGQWRMCTATGLYYGDVIWVILSVEVGGLLGVTQQLSSFETEFNTQPHRKVEGNFNPFASPQKNRQSDENNLKDPGGSEFDSISKNTWAPAPDTWAPAPDQTEQDQNRLSQNSVNLSPSSHANNLSVVTYSKGLHGPYPFGQS

SEQ ID NO: 2 - protein, 298 aa, Gorilla gorilla; identical to SEQ ID NO: 1

SEQ ID NO: 3 - protein, 298 aa, Chlorocebus sabaeus; identical to SEQ ID NO: 1

SEQ ID NO: 4 - protein, 291 aa, Mus musculus
MEGMDVDLDPELMQKFSCLGTTDKDVLISEFQRLLGFQLNPAGCAFFLDMTNWNLQAAIGAYYDFESPNISVPSMSFVEDVTIGEGESIPPDTQFIKTWRIQNSGAEAWPPGVCLKYVGGDQFGHVNMVMVRSLEPQEIADVSVQMCSPSRAGMYQGQWRMCTATGLYYGDVIWVILSVEVGGLLGVTQQLSSFETEFNTQPHRKVEGNFNPFASPQKNRQSDENNLTDPGGSEFDSISKNTWAPVPEQSEQDQDRLSQSSVNLSPSSPANNLSVVTYSKGLHGPYPFGQS

SEQ ID NO: 5 - protein, 291 aa, Myotis lucifugus
MEGMDVDLDPELMQKFSCLGTTDKDVLISEFQRLLGFQLNPAGCAFFLDMTNWNLQAAIGAYYDFESPNISMPSMSFVEDVTIGEGESIPPDTQFIKTWRIQNSGAEAWPPGVCLKYVGGDQFGHVNMVMVRSLEPQEVADVSVQMCSPSRAGMYQGQWRMCTATGLYYGDVIWVILSVEVGGLLGVTQQLSSFETEFNTQPHRKVEGNFNPFASPQKNRQSDENNLKDPGGSEFDSISKNTWAPAPDQTEQDENRLSQNSVNLSPSSHANNLSVVTYSKQQHKTYPFSPP

SEQ ID NO: 6 - protein, 291 aa, Canis familiaris
MEGMDVDLDPELMQKFSCLGTTDKDVLISEFQRLLGFQLNPAGCAFFLDMTNWNLQAAIGAYYDFESPNISVPSMSFVEDVTIGEGESIPPDTQFIKTWRIQNSGAEAWPPGVCLKYVGGDQFGHVNMVMVRSLEPQEIADVSVQMCSPSRAGMYQGQWRMCTATGLYYGDVIWVILSVEVGGLLGVTQQLSSFETEFNTQPHRKVEGNFNPFASPQKNRQSDENNLKDPGGSGFDSISKNTWAPAPDQTEQDENRLSQNSVNLSPSSHANNLSVVTYSKGLHGPYPFGQS

SEQ ID NO: 7 - protein, 291 aa, Gallus gallus
MEGMDVDLDAELMQKFSCLGTTDKDVLIGEFQRLLGFQLSPAGCAFFLDMTNWNLQAAIGAYYDFESPNINVPSMSFVEDVTIGEGESIPPDTQFTKTWRIQNTGTEVWPPGVCLKYVGGDQFGHVNMVMVRSLEPQEIADVSVQMCSPSTAGMYQGQWRMCTATGLYYGDVIWVILSVEVGGLLGVTQQLSSFETEFNTQPHRKVEGNFNPFASPQKNRQPDENNLKDPGGSELGTISKNTWGPAPDQIEQDQNGLSQNSVNLSPSSHSNNLSVVTYSKGFHGPYPFGQS

SEQ ID NO: 8 - protein, 291 aa, Anolis carolinensis
MEGMDVDLDAELMQKFSCLGTTDKDVLIGEFQRLLGFQLSPAGCAFFLDMTNWNLQAAIGAYYDFESPNINVPSMSFVEDVTIGEGESIPPDTQFTKTWRIQNTGAEAWPPGVCLKYVGGDQFGHVNMVMVRSLEPQEIADVSVQMCSPSTAGMYQGQWRMCTATGLYYGDIIWVILSVEVGGLLGVTQQLSSFETEFNTQPHRKVEGNFNPFASPQKNRQPDENNLKDPGGSELDTISKNTWGPAPDQIEQDQTELSQNSVNLSPSSHSNNLSVVTYSKGFHGPYPFGQS

SEQ ID NO: 9 - protein, 280 aa, Xenopus tropicalis
MEGMDVDLDPELMQKFSCLGTTDKDVLISEFQRLLGFQLNPAGCAFFLDMTNCSYNVKAVLFNLYPKQLTAVLLPCIFILYLAMHMLPAVPPDLTITRTWSFTNITGSEAWPPGVCLRYVGGDQFGHVNMVLVRSLDAQEMTDVSVPMCSPSQAGMYQGQWRMCTATGLYFGDVIWVILSVEAGGLLGVTQQLSSFETEFNTQPHRNLGGNYNPFASPQKSRSPDLEDMGTWGPQTEPSENEENGLSQTSVNICAPDHPANCSVATYRESVHGSYPFGES

SEQ ID NO: 10 - protein, 285 aa, Danio rerio
MLMEGMDIDLDQELMQKFSCMGTTDKDVLISEFQRLLGFQLNPAGCAFFLDMTNWNLQAAIGAYYDFESPNINTPSMSFVEDVTIGEGESVPPDTPFTKTWRIQNTGTESWPPGVCLKYVGGDQFGHVNMVMVRSLDPQEISDVSVQMRSPAVPGMYQGQWRMCTATGLFYGDVIWVILSVEEGGLLGVTQQLSSFKTEFNTQPHRSLEGDYNPFASPQKNKQDTNEDHLKDPGGPWEAPLDSIQQDQNGLNHSSVNITPNGLQNNLSVVTYSQGINGPFPFGQS

SEQ ID NO: 11 - protein, 241 aa, Ciona intestinalis
MEETEGVMDVDQCLVGKFSSMGTTDKDVLIDEFRKLLGFQLNAAGCEFFLDMTNWNLHAAIGAYYDFEMPSSKIPSMSFVSDITIGEGEAIPPGTDFIKTWRFQNSGVEKWPLGCTLRFVNGERMSSPEWISVGEVQPHETVDVSVKMRSPINAGLYQGQWRMFTRGMAPFGDIIWVIISVEVGGLLGVTQQMSQFSTGTNNQHQLNTTQNNPFAVDGLGANMDVNVSFGSNNGGQTFDEV

SEQ ID NO: 12 - protein, 41 aa, Homo sapiens
DKDVLISEFQRLLGFQLNPAGCAFFLDMTNWNLQAAIGAYY

SEQ ID NO: 13 - protein, 43 aa, Homo sapiens
  X (7)..(7): S or G or D
  X (10)..(10): Q or R
  X (11)..(11): R or K
  X (18)..(18): N or S
  X (19)..(19): P or A
  X (23)..(23): A or E
  X (31)..(31): C or Absent
  X (32)..(32): S or Absent
  X (33)..(33): W or Y
  X (35)..(35): L or V
  X (36)..(36): Q or K or H
  X (38)..(38): A or V
  X (39)..(39): I or L
  X (40)..(40): G or F
  X (41)..(41): A or N
  X (42)..(42): Y or L
DKDVLIXEFXXLLGFQLXXAGCXFFLDMTNXXXNXXAXXXXXY

SEQ ID NO: 14 - DNA, 27 bp, Artificial Sequence
  Primer
attgagctcgccaccatggaaggaatg

SEQ ID NO: 15 - DNA, 30 bp, Artificial Sequence
  Primer
tagctcgagtcatcatttgtcgtcgtcatc

SEQ ID NO: 16 - DNA, 54 bp, Artificial Sequence
  Primer
ttctcgagtcacttgtcatcgtcatccttgtaatctccatgactagaggggctc

SEQ ID NO: 17 - DNA, 54 bp, Artificial Sequence
  Primer
ttctcgagtcacttgtcatcgtcatccttgtaatctccgctgctcagctgctgg

SEQ ID NO: 18 - DNA, 53 bp, Artificial Sequence
  Primer
ttctcgagtcacttgtcatcgtcatccttgtaatctccgctcattgatggcac

SEQ ID NO: 19 - DNA, 33 bp, Artificial Sequence
  Primer
attgagctcgccaccatgagctttgtggaagac

SEQ ID NO: 20 - DNA, 20 bp, Artificial Sequence
  Primer
ccgtgagtttcccagaagaa

SEQ ID NO: 21 - DNA, 20 bp, Artificial Sequence
  Primer
actgcccaagatgaagacca

SEQ ID NO: 22 - DNA, 20 bp, Artificial Sequence
  Primer
agtaggcgacactgttcgca

SEQ ID NO: 23 - DNA, 20 bp, Artificial Sequence
  Primer
agcctcccattcaattgcca

SEQ ID NO: 24 - DNA, 20 bp, Artificial Sequence
  Primer
acccccaggagaagattcca

SEQ ID NO: 25 - DNA, 21 bp, Artificial Sequence
  Primer
caccaggcaagtctcctcatt

SEQ ID NO: 26 - DNA, 23 bp, Artificial Sequence
  Primer
caacctcctctctgccatcaaga

SEQ ID NO: 27 - DNA, 23 bp, Artificial Sequence
  Primer
ctggaagacccctcccagataga

SEQ ID NO: 28 - DNA, 17 bp, Artificial Sequence
  Primer
cagccatgggctgggac

SEQ ID NO: 29 - DNA, 20 bp, Artificial Sequence
  Primer
tcctcaccaggatgctcaga

SEQ ID NO: 30 - DNA, 20 bp, Artificial Sequence
  Primer
gcaaaatcctgacctggtgt

SEQ ID NO: 31 - DNA, 20 bp, Artificial Sequence
  Primer
gctcgtcctctgtgaactcc

SEQ ID NO: 32 - DNA, 22 bp, Artificial Sequence
  Primer
tgggtgattctcagtgtggagg

SEQ ID NO: 33 - DNA, 22 bp, Artificial Sequence
  Primer
tctaccttacgatgcggctgtg

SEQ ID NO: 34 - DNA, 21 bp, Artificial Sequence
  Primer
ctataaattgagcccgcagcc

SEQ ID NO: 35 - DNA, 20 bp, Artificial Sequence
  Primer
accaaatcggttgactccga

SEQ ID NO: 36 - RNA, 19 nt, Artificial Sequence
  siRNA
acacacagccgcaucguaa

SEQ ID NO: 37 - RNA, 19 nt, Artificial Sequence
  siRNA
gagucaauaccuccggaua

SEQ ID NO: 38 - RNA, 19 nt, Artificial Sequence
  siRNA
ggguggacuuuuaggagua

SEQ ID NO: 39 - RNA, 19 nt, Artificial Sequence
  siRNA
cagcaauuggcgccuauua

SEQ ID NO: 40 - protein, 307 aa, Artificial Sequence
  C6orf106-FLAG protein sequence
MEGMDVDLDPELMQKFSCLGTTDKDVLISEFQRLLGFQLNPAGCAFFLDMTNWNLQAAIGAYYDFESPNISVPSMSFVEDVTIGEGESIPPDTQFVKTWRIQNSGAEAWPPGVCLKYVGGDQFGHVNMVMVRSLEPQEIADVSVQMCSPSRAGMYQGQWRMCTATGLYYGDVIWVILSVEVGGLLGVTQQLSSFETEFNTQPHRKVEGNFNPFASPQKNRQSDENNLKDPGGSEFDSISKNTWAPAPDTWAPAPDQTEQDQNRLSQNSVNLSPSSHANNLSVVTYSKGLHGPYPFGQSGDYKDDDDK

SEQ ID NO: 41 - protein, 285 aa, Artificial Sequence
  C6orf106 (1-276)-FLAG protein sequence
MEGMDVDLDPELMQKFSCLGTTDKDVLISEFQRLLGFQLNPAGCAFFLDMTNWNLQAAIGAYYDFESPNISVPSMSFVEDVTIGEGESIPPDTQFVKTWRIQNSGAEAWPPGVCLKYVGGDQFGHVNMVMVRSLEPQEIADVSVQMCSPSRAGMYQGQWRMCTATGLYYGDVIWVILSVEVGGLLGVTQQLSSFETEFNTQPHRKVEGNFNPFASPQKNRQSDENNLKDPGGSEFDSISKNTWAPAPDTWAPAPDQTEQDQNRLSQNSVNLSPSSHGDYKDDDDK

SEQ ID NO: 42 - protein, 202 aa, Artificial Sequence
  C6orf106 (1-193)-FLAG protein sequence/C6orf106 (delta dis)-FLAG protein sequence
MEGMDVDLDPELMQKFSCLGTTDKDVLISEFQRLLGFQLNPAGCAFFLDMTNWNLQAAIGAYYDFESPNISVPSMSFVEDVTIGEGESIPPDTQFVKTWRIQNSGAEAWPPGVCLKYVGGDQFGHVNMVMVRSLEPQEIADVSVQMCSPSRAGMYQGQWRMCTATGLYYGDVIWVILSVEVGGLLGVTQQLSSGDYKDDDDK

SEQ ID NO: 43 - protein, 85 aa, Artificial Sequence
  C6orf106 (1-76)-FLAG protein sequence
MEGMDVDLDPELMQKFSCLGTTDKDVLISEFQRLLGFQLNPAGCAFFLDMTNWNLQAAIGAYYDFESPNISVPSMSGDYKDDDDK

SEQ ID NO: 44 - protein, 233 aa, Artificial Sequence
  C6orf106 (dUBA)-FLAG protein sequence
MSFVEDVTIGEGESIPPDTQFVKTWRIQNSGAEAWPPGVCLKYVGGDQFGHVNMVMVRSLEPQEIADVSVQMCSPSRAGMYQGQWRMCTATGLYYGDVIWVILSVEVGGLLGVTQQLSSFETEFNTQPHRKVEGNFNPFASPQKNRQSDENNLKDPGGSEFDSISKNTWAPAPDTWAPAPDQTEQDQNRLSQNSVNLSPSSHANNLSVVTYSKGLHGPYPFGQSGDYKDDDDK

SEQ ID NO: 45 - protein, 240 aa, Artificial Sequence
  C6orf106 (dFW)-FLAG protein sequence
MEGMDVDLDPELMQKFSCLGTTDKDVLISEFQRLLGFQLNPAGCAFFLDMTNWNLQAAIGAYYDFESPNISVPSMSFVEDVTIGEGESIPPDTQFVKTWRIQNSDVIWVILSVEVGGLLGVTQQLSSFETEFNTQPHRKVEGNFNPFASPQKNRQSDENNLKDPGGSEFDSISKNTWAPAPDTWAPAPDQTEQDQNRLSQNSVNLSPSSHANNLSVVTYSKGLHGPYPFGQSDYKDDDDK

SEQ ID NO: 46 - DNA, 897 bp, Homo sapiens
atggagggcatggacgtagacctggacccggagctgatgcagaagttcagctgcctgggcaccaccgacaaggacgtgctcatctccgagttccagaggctgctcggcttccagctcaatcctgccggttgcgccttcttcctggacatgaccaactggaacctacaagcagcaattggcgcctattatgactttgagagcccaaacatcagtgtgccctctatgtcctttgttgaagatgtcaccataggagaaggggagtcaatacctccggatactcagtttgtaaaaacatggcggatccagaattctggggcagaggcctggcctccaggggtttgtcttaaatatgtcgggggagaccaatttggacatgtgaacatggtgatggtgagatcgctagagccccaagagattgcagatgtcagcgtccagatgtgcagccccagcagagcaggaatgtatcagggacagtggcggatgtgcactgctacaggactctactatggagatgtcatctgggtgattctcagtgtggaggtgggtggacttttaggagtaacgcagcagctgtcatcttttgaaacggagttcaacacacagccgcatcgtaaggtagaaggaaacttcaacccttttgcctctccccaaaagaaccgacaatcagatgaaaacaacttaaaagaccctgggggctccgagttcgactcgatcagcaaaaacacatgggctcctgctcctgacacatgggctcctgctcctgaccaaactgagcaagaccagaatagactgtcacagaactctgtaaatctgtctcccagcagtcacgcaaacaacttatcagtagtgacttacagtaaggggctccatgggccttaccccttcggccagtcttaa

SEQ ID NO: 47 - DNA, 897 bp, Gorilla gorilla; identical to SEQ ID NO: 46

SEQ ID NO: 48 - DNA, 915 bp, Chlorocebus sabaeus
atggagggcatggacgtggacctggacccggagctgatgcagaagttcagctgcctgggcactaccgacaaggacgtgctcatctccgagttccagaggctgctcggcttccagctcaatcctgccggttgcgccttcttcctggacatgaccaactggaacctccaagcagcaattggcgcctattatgactttgagagcccaaacatcagtgtgccctctatgtcctttgttgaagatgtcaccataggagaaggggagtcaatacctcctgacactcagtttgtaaaaacatggcggatccaaaattctggggcagaggcctggcctccaggggtttgtcttaaatatgtcgggggagaccaatttggacatgtgaacatggtgatggtgagatcgctagagccccaagagattgcagatgtcagcgtccagatgtgcagccccagcagagcaggaatgtatcagggacagtggcggatgtgcactgctacaggactctactatggagatgtcatctgggtgattctcagtgtggaggtgggtggacttttaggagtaacgcagcagctgtcatcttttgaaacggagttcaacacacagccgcatcgtaaggtagaaggaaacttcaacccttttgcctctccccaaaagaaccgacaatcagatgaaaacaacttaaaagaccctgggggctccgagttcgactcgatcagcaaaaacacttgggctcctgctcctgacacatgggctcctgctcctgaccaaactgagcaagaccagaatagactgtcacagaactctgtaaatctgtctcccagcagtcacgcaaacaacttatcagtagtgacttacagtaagacacagccgccagcccgaacagcagcggcatgggcagcgccagcccgggtctga

SEQ ID NO: 49 - DNA, 876 bp, Mus musculus
atggagggcatggacgtggacttggatccggagctcatgcagaagttcagctgcctgggcaccacagacaaggacgtgctcatctccgagttccagcggctactcggcttccagctcaacccggccggctgcgccttcttcctggacatgaccaactggaacttacaagcagcgattggcgcctattatgacttcgagagcccaaatatcagtgtgccctccatgtcctttgttgaagatgtcaccataggagaaggggagtcaataccccctgatacccagttcataaaaacttggcggatacagaattctggagcagaggcctggcctccaggggtttgtcttaaatatgtcgggggagaccagtttggacatgtgaacatggtgatggtgagatcactagagccccaagagatcgcagacgtcagcgtccagatgtgcagtcccagcagagcaggcatgtaccagggacagtggcggatgtgcactgctactggactctactatggagatgtcatctgggtgattctcagtgtggaggtgggtggacttttaggagtaacgcagcagctgtcatcttttgaaacggaattcaacacacagccacatcgcaaggtagaaggaaacttcaacccgtttgcctctccccaaaagaaccgacaatcagatgaaaacaacctaacagaccctgggggttccgagttcgactcgatcagcaaaaatacatgggctcctgttcctgagcaaagcgagcaagatcaggatagactgtcacagagctctgtaaatctgtcccccagcagtcccgcaaacaacttatcagtagtgacttacagtaaggggctccacgggccttaccccttcggccagtcttaa

SEQ ID NO: 50 - DNA, 876 bp, Myotis lucifugus
atggagggcatggacgtggacctggaccccgagctgatgcagaagttcagctgtctgggcaccaccgacaaggacgtgctcatctccgagttccagcggctgctcggcttccagctcaacccggccggctgcgccttcttcctggacatgaccaactggaacctacaagcagcaattggcgcctattatgacttcgagagcccaaacatcagtatgccctctatgtcttttgttgaagatgtcaccatcggagaaggggagtcgatacctcctgatactcagtttataaaaacatggcggatccagaattctggggcagaggcctggcctccaggggtttgtcttaaatatgtcgggggagaccagtttggacatgtgaacatggtgatggtgagatcactagagccccaagaggttgcagacgtcagcgtccagatgtgcagccccagcagagccggaatgtatcagggacagtggcggatgtgcactgctacgggactctactatggagatgtcatctgggtgattctcagtgtggaggtgggtggacttttaggagtaacgcagcagctgtcatctttcgaaacggagttcaacacacagccacatcgcaaggtagaaggaaacttcaacccgtttgcctctccccaaaagaaccgacaatcagatgaaaacaacttaaaagaccctgggggttccgagttcgactcaatcagcaaaaacacatgggctcctgctcctgaccaaaccgagcaagatgagaatagactctcacagaactctgtaaatctgtcccccagcagtcacgcaaacaacttatcagtagtgacttacagtaaggggctccacgggccttaccccttcggccagtcttaa

SEQ ID NO: 51 - DNA, 876 bp, Canis familiaris
atggagggcatggacgtggacctggaccccgagctgatgcagaagttcagctgcttgggcaccaccgacaaggacgtgctcatctccgagttccagcggctcctcggcttccagctcaacccggccggctgcgccttcttcctggacatgaccaactggaacctacaagcagcaattggcgcctattacgactttgagagcccaaacatcagtgtgccctctatgtcatttgttgaagatgtcaccataggagaaggggagtcgatacctcctgatactcaatttataaaaacatggcggatccagaattctggggcagaggcctggcctccaggggtttgtcttaaatatgtcgggggagaccaatttggacatgtgaacatggtgatggtgagatcgctagagccccaggagattgcagatgtcagcgtccagatgtgcagccccagcagagcaggaatgtatcagggacagtggcggatgtgcactgctacaggactctactatggagatgtcatctgggtgattctcagtgtggaggtgggtggacttttaggagtaacgcagcagctgtcatcttttgaaacggagttcaacacacagccacatcgcaaggtagaaggaaacttcaacccgtttgcctctccccaaaagaaccgacaatcagatgaaaacaacttaaaagaccctgggggttccgggttcgactcgatcagcaaaaacacatgggctcctgctcctgaccaaaccgagcaagatgagaatagactctcacagaactctgtaaatctgtcccccagcagtcacgcaaacaacttatcagtagtgacttacagtaaggggctccacgggccttaccccttcggccagtcttaa

SEQ ID NO: 52 - DNA, 876 bp, Gallus gallus
atggagggcatggacgtggatttggacgcggagctgatgcagaagttcagctgcttgggtactaccgacaaggacgtgctgatcggcgagttccagcggctcctcggcttccagcttagcccggccggctgcgccttcttcctcgacatgaccaactggaatctacaagccgccattggagcctattatgattttgagagtccaaacatcaacgtaccctccatgtcctttgttgaagatgtcaccataggtgaaggagagtccattcctcctgacacccagtttacgaaaacatggaggatacaaaacacagggacagaagtatggcctccaggagtttgtctgaagtacgttgggggagaccagtttggccatgtaaacatggtaatggtcaggtccctggagccccaggagattgcagatgtcagcgttcagatgtgcagccccagcacagcaggaatgtatcagggacagtggcgaatgtgcactgccacaggactgtattacggagatgtcatctgggtgatcctcagtgtggaagttggaggacttctaggagtaacacagcagctgtcatcctttgaaacggagttcaacacacaaccgcaccgcaaggtagaaggaaactttaacccattcgcctctccacagaagaacaggcaaccagatgaaaacaacctaaaagaccctgggggttctgagctaggcacaatcagcaaaaacacgtgggggcctgctcctgaccaaattgaacaagatcagaatggactgtcacaaaactctgtaaatctctcccccagcagtcactcgaacaacttgtcggtagtgacatacagtaagggtttccacggtccttatcccttcggacagtcttaa

SEQ ID NO: 53 - DNA, 876 bp, Anolis carolinensis
atggaggggatggacgtggacttggacgcggagctgatgcagaagttcagctgcctgggcaccaccgacaaggacgtgctcatcggggagttccagcgcctgctcggcttccaactcagccccgccggatgcgccttcttcctggacatgacgaactggaacttacaagcagccattggagcctactatgattttgagagtccaaacatcaacgtgccctccatgtccttcgtagaagatgtgaccataggagaaggagagtccattccccctgacacccagtttacaaaaacatggaggatacagaacacaggggcagaagcatggcctccaggtgtttgtcttaaatacgtcgggggagaccagtttggccatgtgaacatggtgatggtccgatcattggagccccaggagattgcagatgtcagcgttcagatgtgcagccccagtactgcaggaatgtatcaaggacagtggcgaatgtgtactgccacaggactctactatggagacatcatctgggtgatcctcagtgtggaagttggaggacttttaggagtaacacagcagctgtcatcatttgaaacagagttcaacacgcaaccacatcgcaaggtagaaggaaactttaacccattcgcctctccgcagaagaacaggcaaccagatgaaaacaacttaaaagaccctgggggttccgagctagatacaatcagcaaaaacacgtgggggcctgctcctgaccaaattgaacaagatcagactgaactatcacaaaactctgtaaatctctcccccagcagtcactcaaacaacttatcagtagtgacatacagtaagggttttcatggtccttacccctttggacagtcttaa

SEQ ID NO: 54 - DNA, 837 bp, Xenopus tropicalis
atggaggggatggacgttgatcttgacccagagctcatgcagaagttcagttgtttggggactactgataaggatgttttgatttcagaatttcagcgccttcttggttttcagctcaaccctgctggctgcgcttttttcctagacatgactaactggaatctccaagctgcaattggagcatattatgactttgaaagcccaagtgtcactgtcccatgcatgtcatttgtagaagatgtaactattggggagggggaatctgtgccaccagacacccagtttaccaagacctggagaatccagaacacaggatcagaggcatggccccctggtgtgtgtctgcgttatgtagggggtgatcagtttggtcatgtcaacatggtattagtccgatccctagatgcacaggagatgactgatgtcagtgtgccaatgtgcagtccaagccaggccggcatgtaccagggccagtggaggatgtgtactgctactggtctttattttggagatgttatttgggtgatactgagtgtagaggctggaggcctattgggagttactcagcaactgtcttcattcgaaacagaatttaatactcaacctcatcgaaatcttggaggcaactacaacccctttgcttctccacagaaaagccggtcacctgatctggaggacatgggtacatgggggcctcagacagagccctctgaaaatgaggagaatggactttctcagacctctgtaaatatatgtgcaccggatcacccggccaactgttcagttgcaacatatagagagagtgtgcatgggtcttacccttttggagaatcttaa

SEQ ID NO: 55 - DNA, 858 bp, Danio rerio
atgctcatggagggcatggacatagacctggatcaggagctcatgcaaaaattcagctgcatgggcaccacggataaagatgtcctcatctcggagttccagaggctgctgggatttcagctcaacccggctggctgcgccttcttcctggacatgaccaactggaaccttcaagcagctattggtgcttattatgactttgagagtccaaacatcaacacgccgtcaatgtcttttgtggaagacgtgacgattggagagggggagtctgttcctcctgacacaccgttcacaaagacatggaggatacagaacacaggtacagagtcgtggcccccaggtgtgtgtctgaagtatgtgggcggggatcagtttggtcatgtgaacatggtaatggtgcggtctctggaccctcaggaaatatcagatgtgagtgtgcagatgcgtagtccagctgttcctggcatgtaccagggccagtggagaatgtgcacagccacaggactcttctacggagatgtgatctgggtgattttgagtgtggaggagggaggcctgctgggcgtcacacagcagctatcctccttcaagacggagttcaacacgcagccacaccgcagtctggagggagactacaaccctttcgcctcaccacagaagaacaagcaggacaccaatgaggatcatctaaaagaccctgggggcccctgggaggccccactggactccattcagcaagatcaaaatggactcaatcacagctctgtaaatattacaccaaatggtctccaaaacaacttatcagtagtgacttacagccagggcattaatggacccttcccgtttggacagtcttaa

SEQ ID NO: 56 - DNA, 1092 bp, Ciona intestinalis
atggaagaaaccgagggcgtgatggatgtagaccaatgtttggtcgggaagtttagttcaatgggcacgaccgataaagatgttttaatcgatgagtttcgtaaacttctgggtttccaacttaacgctgcaggttgcgagttctttctagacatgacaaactggaacttacacgcggcgattggcgcttattatgacttcgagatgccttcgagtaaaatcccttcaatgtcgttcgttagtgacatcacaataggagagggggaagcgattccaccaggaacagattttataaaaacctggagatttcagaattcgggggtcgagaagtggccactaggttgcacccttcgcttcgttaacggggagcgaatgagttcccccgagtggatatcggtgggcgaggtccaacctcatgaaaccgtcgatgtttcagtgaaaatgcgaagtccgataaacgctggattgtaccagggtcagtggaggatgtttacgaggggaatggcgccattcggtgatattatctgggtgatcatatcagtagaggtgggtgggttgttgggggtcacgcaacaaatgtcacaattctccaccgggacgaacaaccaacatcagttgaatacgacgcagaacaatccattcgctgtggatggattgggagcaaacatggacgttaatgtttcatttgggagcaataatggcggacaaacatttgatgaagttccgcggaagtgtgaacgaggtacagcacatgacgtgtcgtcctgttgggaaacgaaaagtccgaccttgacaatggaagagggtttgggcggggaaggccacaccctgtgtcggatcgatgaagaaacaacgaacgatgggaataataacgagggagtgaaatatctttcgtcaaaatcctcgtacgacctcgcagctcgtcaagcgtcacctttcacatcaccctccaaactcgttgacctggaacgatcgttctcgaccccagatgccccttcacccattcacccaacaaccaatctactttcaataaatttccaggttggttgcttttatctcaactacttcagtcataacagttgtcaaacatag

SEQ ID NO: 57 - protein, 93 aa, Artificial Sequence
  FW domain of the H. sapiens C6orf106 protein sequence
TWRIQNSGAEAWPPGVCLKYVGGDQFGHVNMVMVRSLEPQEIADVSVQMCSPSRAGMYQGQWRMCTATGLYYGDVIWVILSVEVGGLLGVTQQ

SEQ ID NO: 58 - protein, 94 aa, Artificial Sequence
  Consensus sequence of the C6orf106 FW domain
  misc_feature (3)..(5): Xaa can be any naturally occurring amino acid
  misc_feature (7)..(8): Xaa can be any naturally occurring amino acid
  misc_feature (10)..(10): Xaa can be any naturally occurring amino acid
  misc_feature (12)..(12): Xaa can be any naturally occurring amino acid
  misc_feature (15)..(15): Xaa can be any naturally occurring amino acid
  misc_feature (17)..(18): Xaa can be any naturally occurring amino acid
  misc_feature (20)..(21): Xaa can be any naturally occurring amino acid
  misc_feature (23)..(23): Xaa can be any naturally occurring amino acid
  misc_feature (25)..(34): Xaa can be any naturally occurring amino acid
  misc_feature (36)..(41): Xaa can be any naturally occurring amino acid
  misc_feature (43)..(44): Xaa can be any naturally occurring amino acid
  misc_feature (49)..(49): Xaa can be any naturally occurring amino acid
  misc_feature (51)..(51): Xaa can be any naturally occurring amino acid
  misc_feature (54)..(56): Xaa can be any naturally occurring amino acid
  misc_feature (58)..(58): Xaa can be any naturally occurring amino acid
  misc_feature (66)..(66): Xaa can be any naturally occurring amino acid
  misc_feature (68)..(73): Xaa can be any naturally occurring amino acid
  misc_feature (76)..(76): Xaa can be any naturally occurring amino acid
  misc_feature (81)..(81): Xaa can be any naturally occurring amino acid
  misc_feature (85)..(85): Xaa can be any naturally occurring amino acid
TWXXXNXXGXEXWPXGXXLXXVXGXXXXXXXXXXVXXXXXXEXXDVSVXMXSPXXXGXYQGQWRMXTXXXXXXGDXIWVIXSVEXGGLLGVTQQ

SEQ ID NO: 59 - protein, 191 aa, Artificial Sequence
  C6(deltaFW)-FLAG protein sequence
MEGMDVDLDPELMQKFSCLGTTDKDVLISEFQRLLGFQLNPAGCAFFLDMTNWNLQAAIGAYYDFESPNISVPSMSLSSFETEFNTQPHRKVEGNFNPFASPQKNRQSDENNLKDPGGSEFDSISKNTWAPAPDTWAPAPDQTEQDQNRLSQNSVNLSPSSHANNLSVVTYSKGLHGPYPFGQSDYKDDDK

SEQ ID NO: 60 - protein, 117 aa, Artificial Sequence
  FW domain of the H. sapiens C6orf106 protein sequence
SMSFVEDVTIGEGESIPPDTQFVKTWRIQNSGAEAWPPGVCLKYVGGDQFGHVNMVMVRSLEPQEIADVSVQMCSPSRAGMYQGQWRMCTATGLYYGDVIWVILSVEVGGLLGVTQQ

SEQ ID NO: 61 - protein, 117 aa, Artificial Sequence
  Disordered region of the H. sapiens C6orf106 protein sequence
GGLLGVTQQLSSFETEFNTQPHRKVEGNFNPFASPQKNRQSDENNLKDPGGSEFDSISKNTWAPAPDTWAPAPDQTEQDQNRLSQNSVNLSPSSHANNLSVVTYSKGLHGPYPFGQS

SEQ ID NO: 62 - protein, 108 aa, Artificial Sequence
  Disordered region of the H. sapiens C6orf106 protein sequence
LSSFETEFNTQPHRKVEGNFNPFASPQKNRQSDENNLKDPGGSEFDSISKNTWAPAPDTWAPAPDQTEQDQNRLSQNSVNLSPSSHANNLSVVTYSKGLHGPYPFGQS